(19) United States
(12) Patent Application Publication
(10) Pub. No.: US 2007/0031850 A1
(43) Pub. Date: Feb. 8, 2007

(54) Nucleic acid arrays for detecting multiple strains of a non-viral species

(76) Inventors: William Martin Mounts, Andover, MA (US); Maryann Zinni Whitley, Quincy, MA (US); Ellen Murphy, City Island, NY (US)

Correspondence Address: NIXON PEABODY, LLP, 401 9TH STREET, NW, SUITE 900, WASHINGTON, DC, 20004-2128, US

(21) Appl. No.: 10/859,198

(22) Filed: Jun. 3, 2004

Related U.S. Application Data
(60) Provisional application No. 60/475,871, filed on Jun. 5, 2003.

(51) Int. Cl. C12Q 1/68; C12M 1/34

(52) U.S. Cl. 435/6; 435/287.2

(57) ABSTRACT

Nucleic acid arrays and methods of using the same for concurrent or discriminable detection of different strains of a non-viral species. In many embodiments, the nucleic acid arrays of the present invention include probes that are specific to different respective strains of a non-viral species. In many other embodiments, the nucleic acid arrays of the present invention include probes that are common to two or more different strains of the non-viral species. In one embodiment, the non-viral species is Staphylococcus aureus, and the different Staphylococcus aureus strains include COL, N315, Mu50, EMRSA-16, MSSA-476, and 8325 strains. In another embodiment, a nucleic acid array of the present invention includes polynucleotide probes capable of hybridizing under stringent or nucleic acid array hybridization conditions to respective sequences selected from SEQ ID NOs: 1 to 7,852, or the complements thereof.

### CROSS-REFERENCE TO RELATED APPLICATIONS

The present application claims priority from and incorporates by reference the entire content of U.S. Provisional Patent Application Ser. No. 60/475,871, filed Jun. 5, 2003.

This application incorporates by reference all materials on the compact discs labeled “Copy 1” and “Copy 2.” Each of the compact discs includes the following files: Table A.txt (667 KB, created on May 18, 2004), Table B.txt (671 KB, created on May 18, 2004), Table C.txt (1,326 KB, created on May 18, 2004), Table D.txt (151 KB, created on May 18, 2004), Table E.txt (153 KB, created on Jun. 2, 2004), Table F.txt (3,273 KB, created on May 18, 2004), Table G.txt (9,518 KB, created on Jun. 2, 2004), and “AM101085 Sequence Listing.ST25.txt” (53,562 KB, created on May 26, 2004).

LENGTHY TABLES FILED ON CD
The patent application contains a lengthy table section. A copy of the table is available in electronic form from the USPTO web site (http://seqdata.uspto.gov/?pageRequest=docDetail&DocID=US20070031850A1) An electronic copy of the table will also be available from the USPTO upon request and payment of the fee set forth in 37 CFR 1.19(b)(3).

### TECHNICAL FIELD

This invention relates to nucleic acid arrays and methods of using the same for concurrent or discriminable detection of different strains of Staphylococcus aureus or other non-viral species.

### BACKGROUND

Staphylococcus aureus is a leading cause of soft tissue infections. It can cause conditions such as pneumonia, meningitis, skin conditions (e.g. acne, boils or cellulites), arthritis, osteomyelitis, endocarditis, urinary tract infections, and toxic shock syndrome. Some strains of Staphylococcus aureus produce enterotoxins which cause staphylococcal food poisoning (staphyloenterotoxicosis or staphyloenterotoxemia). The most common symptoms for staphylococcal food poisoning include nausea, vomiting, retching, abdominal cramping, and prostration.

Traditional methods for detecting Staphylococcus aureus involve first growing the bacteria from a sample and then determining the identity of the bacteria. Examples of these methods include the direct plate count method and the most probable number (MPN) method. U.S. Patent Application Publication No. 20020055101 describes a PCR-based method for detecting Staphylococcus aureus. These traditional and PCR-based methods, however, are incapable of discriminably detecting multiple strains of Staphylococcus aureus at the same time.

Therefore, one object of this invention is to provide systems and methods which allow for concurrent and discriminable detection of different strains of Staphylococcus aureus or other non-viral species.

### SUMMARY OF THE INVENTION

In one aspect, the present invention provides nucleic acid arrays which allow for concurrent or discriminable detection of different strains of a non-viral species. The nucleic acid arrays include a plurality of polynucleotides, each of which is specific to a different respective strain of a non-viral species. In many embodiments, the nucleic acid arrays further include probes that are common to two or more different strains of the non-viral species.

In one embodiment, the non-viral species is Staphylococcus aureus. Examples of Staphylococcus aureus strains that are amenable to the present invention include, but are not limited to, COL, N315, Mu50, EMRSA-16, MSSA-476, MW2, and 8325.

In another embodiment, a nucleic acid array of the present invention includes at least 2, 5, 10, 100, 500, 1,000, 2,000, 3,000, 4,000, or more polynucleotide probes, each of which is capable of hybridizing under stringent or nucleic acid array hybridization conditions to a different respective sequence selected from SEQ ID NOs: 1 to 7,852, or the complement thereof.

In still another embodiment, a nucleic acid array of the present invention includes polynucleotide probes for each sequence selected from SEQ ID NOs: 1 to 7,852, or the complement thereof.

In yet another embodiment, a nucleic acid array of the present invention includes at least six polynucleotide probes, each of which is specific to a different respective Staphylococcus aureus strain selected from the group consisting of COL, N315, Mu50, EMRSA-16, MSSA-476, and 8325.

In many embodiments, a nucleic acid array of the present invention includes two groups of polynucleotide probes. The first group of probes is capable of hybridizing under stringent or nucleic acid array hybridization conditions to respective sequences selected from SEQ ID NOs: 3,817 to 7,852, or the complements thereof. The second group of probes is capable of hybridizing under stringent or nucleic acid array hybridization conditions to respective sequences selected from SEQ ID NOs: 1 to 3,816, or the complements thereof. Each group can include, without limitation, at least 10, 20, 50, 100, 200, 500, 1,000, or more different probes.

In another embodiment, a nucleic acid array of the present invention includes at least 2, 5, 10, 100, 10, 100, 500, 1,000, 2,000, 3,000, 4,000, or more polynucleotide probes, each of which is capable of hybridizing under stringent or nucleic acid array hybridization conditions to a different respective tiling sequence selected from SEQ ID NOs: 7,704, or the complement thereof.

In one example, a nucleic acid array of the present invention includes probes selected from SEQ ID NOs: 15,737. In another example, the nucleic acid array includes a mismatch probe for each perfect match probe. In yet another example, the nucleic acid array includes probes for virulence genes, antimicrobial resistance genes, multilocus sequence typing genes, leukotoxin genes, agrB genes, or genes encoding ribosomal proteins.

In another aspect, the present invention provides methods that are useful for typing, detecting, or monitoring gene expression of a strain of a non-viral species. The methods include preparing a nucleic acid sample from a sample of interest, and hybridizing the nucleic acid sample to a nucleic acid array of the present invention.

In yet another aspect, the present invention provides methods for preparing nucleic acid arrays. The methods includes selecting a plurality of polynucleotides, each of which is specific to a different respective strain of a non-viral species, and stably attaching the selected polynucleotides to respective regions on one or more substrate supports. The non-viral species can be, without limitation, Staphylococcus aureus or other bacteria. In one embodiment, the methods further include selecting a polynucleotide probe which is common to all of the different strains that are being investigated, and stably attaching the common polynucleotide probe to a discrete region on the substrate support(s). In another embodiment, the methods include identifying a plurality of open reading frames in the genomic sequences of different strains of a non-viral species, and selecting polynucleotide probes for the open reading frames thus identified.

In still another aspect, the present invention provides polynucleotide collections. The polynucleotide collections include at least one polynucleotide capable of hybridizing under stringent or nucleic acid array hybridization conditions to a respective sequence selected from SEQ ID NOs: 1 to 7,852, or the complement thereof.

The present invention also features protein arrays capable of concurrent or discriminable detection of different strains of a non-viral species. The protein arrays include probes that are specific to respective strains of a non-viral species. These probes can specifically bind to respective proteins of the non-viral species.

Other features, objects, and advantages of the present invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings are provided for illustration, not limitation.

FIG. 1 depicts the color scale of the expression level of a gene relative to the mean value for that gene over all nucleic acid arrays that are being investigated.

FIG. 2 shows an unsupervised hierarchical clustering of the normalized profiles of 2,059 “imperfect ORFs” across a set of Staphylococcus aureus strains or clones.

FIG. 3 illustrates the normalized profiles of seven multilocus sequence typing (MLST) genes across a set of Staphylococcus aureus strains or clones.

FIG. 4 shows the normalized profiles of 259 virulence genes across a set of Staphylococcus aureus strains or clones.

FIG. 5 indicates the normalized profiles of Panton-Valentine leukocidin (PVL) genes and other leukotoxin genes across a set of Staphylococcus aureus strains or clones.

FIG. 6 depicts the relationship between the PVL profiles and the profiles of two types of agrB gene.

FIG. 7 shows the normalized profiles of exfoliative toxin A gene (“eta”) and exfoliative toxin B gene (“etb”) across a set of Staphylococcus aureus strains or clones.

FIG. 8A illustrates a nucleic acid array-derived dendrogram (top) with heatmap (beneath) for all qualifiers that were analyzed in each strain. The dendrogran indicates the relatedness of each strain based on the signal intensity of each qualifier across all strains. Within the heatmap, each qualifier is shown vertically for each strain. Red indicates high signal intensity; green indicates low signal intensity. The order of qualifiers is identical for all strains. Scanning horizontally identifies qualifiers that have high signal intensity (red) in some strains but low intensities (green) in others.

FIG. 8B is a dendrogram of CDC strains 10, 13, 12, 9, and 8, which were all considered to be identical strains by both ribotyping and PFGE. Heatmap illustrates 36 qualifiers (horizontally) that are considered present in strains 10 and 13 but absent in other strains, based on adjusted call-determinations.

FIG. 8C shows growth characteristics of CDC strains 10, 13, 12, 9, and 8 on kanamycin-containing agar plates.

### DETAILED DESCRIPTION

The present invention provides nucleic acid arrays which allow for concurrent or discriminable detection of different strains of a non-viral species. In many embodiments, the nucleic acid arrays of the present invention include at least two probes, each of which is specific to a different respective strain of a non-viral species. In many other embodiments, the nucleic acid arrays of the present invention include at least one probe which is common to two or more different strains of a non-viral species. Examples of non-viral species that are amenable to the present invention include, but are not limited to, bacteria, fungi, animals, plants, or other prokaryotic or eukaryotic species. In one embodiment, the non-viral species is a pathogenic microorganism, such as a bacterium or fungus.

Different strains of a non-viral species can have different genetic properties. These genetic differences can be manifested in gene expression profiles and therefore become detectable by using the nucleic acid arrays of the present invention. The present invention contemplates detection of non-viral strains that have distinguishable phenotypical properties, such as immunological, morphological, or antibiotic-resistance properties. The present invention also contemplates detection of non-viral strains that have no distinguishable phenotypical properties. As used herein, “strain” includes subspecies.

The following subsections focus on nucleic acid arrays which allow for concurrent or discriminable detection of different Staphylococcus aureus strains. As appreciated by one of ordinary skill in the art, the same methodology can be readily adapted to the making of nucleic acid arrays that are suitable for the detection of different strains of other non-viral species. The use of subsections is not meant to limit the invention; each subsection may apply to any aspect of the invention. In this application, the use of “or” means “and/or” unless stated otherwise

### A. IDENTIFICATION OF OPEN READING FRAMES AND INTERGENIC SEQUENCES OF STAPHYLOCOCCUS AUREUS STRAINS

Open reading frames (ORFs) and intergenic sequences of different Staphylococcus aureus strains can be derived from their genomic sequences. Numerous Staphylococcus aureus genomes are available from a variety of sources. Table 1 lists six exemplary Staphylococcus aureus strains and the sources from which their genomic sequences can be obtained.

TABLE 1
Genomes of Staphylococcus aureus Strains

| Strain Name | Genome Status | Source |
|---|---|---|
| COL | Complete | The Microbial Database at The |
| | | Institute for Genome Research |
| | | (TIGR) |
| N315 | Complete | GenBank |
| Mu50 | Complete | GenBank |
| EMRSA-16 | Complete | Sanger Centre (United Kingdom) |
| MSSA-476 | Incomplete | Sanger Centre (United Kingdom) |
| 8325 | Incomplete | Oklahoma University |

The incomplete genomes (such as the MSSA-476 and 8325 genomes) can be organized and oriented based on alignments to the complete genomes. The organized and oriented sequence fragments for each incomplete genome can be further bridged with a six-frame stop sequence (such as CTAACTAATFAG).

ORFs in each of the six genomic sequences can be predicted or isolated by various methods. Exemplary methods include, but are not limited to, GeneMark (such as GeneMark 1.2.4a, provided by the European Bioinformatics Institute), Glimmer (such as Glimmer 2.0, provided by TIGR), and ORF Finder (provided by the National Center for Biotechnology Information (NCBI)). In addition, ORF sets can be collected from other sources. For instance, a number of ORF sets in the COL, N315 and Mu50 genomes have been published or publicly disclosed. ORFs present in GenBank or other sequence databases can also be collected.

tRNA and rRNA sequences can be similarly obtained. In one embodiment, tRNA and rRNA identified in the N315 and Mu50 genomes are collected.

The ORFs and other transcribeable sequences thus collected can be separated based on whether they are oriented 5′ to 3′ on the sense or antisense strand of their respective genomes. The strand assignment can be arbitrary. In one embodiment, all of the six genomes described in Table 1 are assigned in a similar manner. That is, the genomes for each of the six Staphylococcus aureus strains are highly conserved such that the overall primary structure is similar. Each genome can be oriented similarly such that the sense and antisense strands between different strains are highly conserved.

The collection of sense and antisense ORFs can then be clustered separately to identify highly homologous ORFs. Separate clustering may prevent the ORFs, which overlap on both the sense and antisense strands, from clustering together. This reduces the chance of generating misleading sequence clusters. Suitable clustering algorithms for this purpose include, but are not limited to, the CAT (cluster and alignment tool) software package provided by DoubleTwist. See Clustering and Alignment Tools User's Guide (DoubleTwist, Inc., 2000).

The CAT program can cause all similar ORFs to cluster together, and then align those similar ORFs to generate one or more sub-clusters. Each sub-cluster of two or more members generates a consensus sequence. The consensus sequences can be generated such that any base ambiguity would be identified with the respective IUPAC (International Union of Pure and Applied Chemistry) base representation, which is consistent with the WIPO Standard ST.25 (1998).

The consensus sequences, in addition to all singleton sequences that are either excluded in the initial clustering or sub-clustered into a singleton sub-cluster, can be manually curated to verify cluster membership. At this stage, some clusters can be joined or separated based on known homologies that are not identified with CAT. Moreover, filtered intergenic sequences can be added to the final set of sequences which are used for generating the nucleic acid array probes.

Examples of the consensus sequences identified using the above-described method are depicted in SEQ ID NOs: 1-3,816. Each of these consensus sequences has a header which includes the identification number (the number after “wyeSaureus2a:”) and other information of the sequence. See Table A. These consensus sequences were derived from sixteen sequence sets that comprised the input sequences for the clustering. These sixteen sequence sets include three sets derived from the COL genome (GeneMark, Glimmer, and TIGR), two sets from each of the 8325, MRSA, and MSSA genomes (GeneMark and Glimmer), three sets from each of the Mu50 and N315 genomes (GeneMark, Glimmer, and public ORF sets), and one set of other GenBank sequences. If a sequence was not derived from the genomes of the six strains listed in Table 1, the sequence belongs to the “Other” category. See Table E.

The consensus sequences represent ORFs or other transcribeable elements that are highly conserved among two or more different input sequences. Some consensus sequences are specific for a single genome and represent the Glimmer, Genemark, and public ORF calls on a single genome. Table E shows the Staphylococcus aureus strains (including the “Other” category) from which each consensus sequence was derived. For example, SEQ ID NO: 7 (consensus:wyeSaureus2a: WAN014A7L-5_at) was derived from and is highly conserved among all of the six strains listed in Table 1, and SEQ ID NO: 1 (consensus:wyeSaureus2a:AB047088-cds7_s_at) was derived from and is conserved among two or more different sequences in the “Other” category. See Table E. The consensus sequences can be used to prepare probes that are common to the Staphylococcus aureus strains from which the sequences were derived.

As used herein, a polynucleotide probe is “common” to a group of strains if the polynucleotide probe can hybridize under stringent conditions to each and every strain selected from the group. A polynucleotide can hybridize to a strain if the polynucleotide can hybridize to an RNA transcript, or the complement thereof, of the strain. In many embodiments, a probe common to a group of strains can hybridize under stringent conditions to a protein-coding sequence (e.g., an exon or the protein-coding region of an mRNA), or the complement thereof, of each strain in the group. In many other embodiments, a probe common to a group of strains does not hybridize under stringent conditions to RNA transcripts, or the complements thereof, of other strains of the same species or strains of other species.

“Stringent conditions” are at least as stringent as, for example, conditions G L shown in Table 2. In certain embodiments of the present invention, highly stringent conditions A-F can be used. In Table 2, hybridization is carried out under the hybridization conditions (Hybridization Temperature and Buffer) for about four hours, followed by two 20-minute washes under the corresponding wash conditions (Wash Temp. and Buffer).

TABLE 2
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4XSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | DNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between | | | | |
| # 18 and 49 base pairs in length, Tₘ(° C.) = 81.5 + 16.6(log₁₀Na⁺) + 0.41(% G + C) − (600/N), where N is the number of bases in the hybrid, and Na⁺ is the molar concentration of sodium ions in the hybridization buffer (Na⁺ for 1xSSC = 0.165M). | | | | |

Examples of the singleton sequences identified using the above-described clustering method, as well as a filtered set of N315 intergenic sequences, are depicted in SEQ ID NOs: 3,852. These sequences are herein referred to as “exemplar” sequences. The same sixteen sequence sets were used to derive both the exemplar sequences in Table B and the consensus sequences in Table A. Each exemplar sequence has a header which includes the identification number (the number after “wyeSaureus2a:”) and other information of the sequence. See Table B.

Many of the singleton sequences are unique to only one Staphylococcus aureus strain listed in Table 1 (e.g., SEQ ID NOs: 4,434), or to only one sequence in the “Other” category (e.g., SEQ ID NOs: 7,852). Some of the singleton sequences are present in more than one genome, but were not called as ORFs and were therefore not in the input sequence set.

Table E illustrates the respective strain from which each exemplar sequence was derived. The exemplar sequences can be used to prepare probes that are specific to the respective Staphylococcus aureus strains from which these sequences were derived. As used herein, a polynucleotide probe is “specific” to a strain selected from a group of strains if the polynucleotide probe is capable of hybridizing under stringent conditions to an RNA transcript, or the complement thereof, of the strain, but is incapable of hybridizing under the same conditions to RNA transcripts, or the complements thereof, of other strains in the group. In many embodiments, a probe specific for a strain can hybridize under stringent conditions to a protein-coding sequence (e.g., an exon or the protein-coding region of an mRNA), or the complement thereof, of the strain, but not RNA transcripts, or the complements thereof, of other strains of the same species or strains of other species. SEQ ID NOs: 4,830 include intergenic sequences, rRNAs, tRNAs, unidentified ORFs, predicted or known ORFs, or other expressible features.

As appreciated by one of ordinary skill in the art, ORFs and other expressible sequences can be similarly extracted from the genomic sequences of other Staphylococcus aureus strains (such as strain MW2, T. Baba, et al., THE LANCET, 359: 1819-1827 (2002)), or strains of other non-viral species. The extracted sequences can be clustered to obtain consensus and singleton sequences. Probes common to two or more strains or probes specific to a particular strain can be derived from the consensus or singleton sequences, respectively. Like Staphylococcus aureus, the genomic sequences of other non-viral strains can be collected from publicly available sequence databases. For instance, the Entrez Genome database at the NCBI provides the genomic sequences for various bacterial strains or subspecies (see, e.g., www.ncbi.nlm.nih.gov/PMGifs/Genomesteub_g.html). These bacterial strains include, but are not limited to, Escherichia coli strains CTF073, K12, O157:H7, and O157:H7 EDL933; Chlamydophila pneumoniae strains CWL029, AR39, and J138; Streptococcus pneumoniae strains R6 and TIGR4; and Streptococcus pyogenes strains MGAS315, MGAS8232, SSI-1, and M1 GAS.

### B. PREPARATION OF POLYNUCLEOTIDE PROBES FOR DETECTING VARIOUS STAPHYLOCOCCUS AUREUS STRAINS

The consensus and exemplar sequences depicted in SEQ ID NOs: 1-7,852 (collectively referred to as the “parent sequences”) can be used for preparing polynucleotide probes. The probes for each parent sequence can hybridize under stringent or nucleic acid array hybridization conditions to the parent sequence, or the complement thereof. In many embodiments, the probes for each parent sequence are incapable of hybridizing under stringent or nucleic acid array hybridization conditions to other parent sequences, or the complements thereof. In one embodiment, the probes for each parent sequence comprise or consist of a sequence fragment of the parent sequence, or the complement thereof.

As used herein, “nucleic acid array hybridization conditions” refer to the temperature and ionic conditions that are normally used in nucleic acid array hybridization. These conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6×SSPET. 6×SSPET contains 0.9 M NaCl, 60 mM NaH₂PO₄, 6 mM EDTA, and 0.005% Triton X-100. Under more stringent nucleic acid array hybridization conditions, the wash buffer can contain 100 mM MES, 0.1 M [Na⁺], and 0.01% Tween 20.

The probes of the present invention can be DNA, RNA, or PNA (“Peptide Nucleic Acid”). Other modified forms of DNA, RNA, or PNA can also be used. The nucleotide units in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes of the present invention can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

In one embodiment, the probes have relatively high sequence complexity. In many instances, the probes do not contain long stretches of the same nucleotide. In another embodiment, the probes can be designed such that they do not have a high proportion of G or C residues at the 3′ ends. In yet another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Any part of a parent sequence can be used to prepare probes. For instance, probes can be prepared from the protein-coding region, the 5′ untranslated region, or the 3′ untranslated region of a parent sequence. Multiple probes, such as 5, 10, 15, 20, 25, 30, or more, can be prepared for each parent sequence. The multiple probes for the same parent sequence may or may not overlap each other. Overlap among different probes may be desirable in some assays.

In many embodiments, the probes for a parent sequence have low sequence identities with other parent sequences, or the complements thereof. For instance, each probe for a parent sequence can have no more than 70%, 60%, 50% or less sequence identity with other parent sequences, or the complements thereof. This reduces the risk of undesired cross-hybridization. Sequence identity can be determined using methods known in the art. These methods include, but are not limited to, BLASTN, FASTA, and FASTDB. The GCG program can also be used, which is a suite of programs including BLASTN and FASTA.

The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the Genetics Computer Group (GCG).

In one embodiment, the parent sequences with large sizes are divided into shorter sequence segments to facilitate the probe design. These shorter sequence segments, together with the remaining undivided parent sequences, are collectively referred to as the “tiling” sequences (SEQ ID NOs: 7,704). Like the parent sequences, each tiling sequence has a header which includes the identification number (the number after “wyeSaureus2a:”) and other information of the tiling sequence. See Table C. Table D shows the location of each tiling sequence in the corresponding parent sequence from which the tiling sequence is derived. “TilingStart” denotes the 5′ end location of a tiling sequence in the corresponding parent sequence, and “TilingEnd” represents the 3′ end location of the tiling sequence.

Polynucleotide probes can be derived from the tiling sequences. The probes for each tiling sequence can hybridize under stringent or nucleic acid array hybridization conditions to that tiling sequence, or the complement thereof. In many embodiments, the probes for each tiling sequence are incapable of hybridizing under stringent or nucleic acid array hybridization conditions to other tiling sequences, or the complements thereof.

Polynucleotide probes for each tiling sequence can be generated using Array Designer, a software package provided by TeleChem International, Inc (Sunnyvale, Calif. 94089). Examples of the polynucleotide probes thus generated are depicted in SEQ ID NOs: 15,737. The 5′ and 3′ ends of each probe in the corresponding tiling sequence are illustrated in Table F (“5′ End” and “3′ End,” respectively). Each probe in Table F can hybridize under stringent or nucleic acid array hybridization conditions to the complement of the corresponding tiling sequence. Other methods or software programs can also be used to prepare probes for the tiling sequences of the present invention.

In one embodiment, perfect mismatch probes are prepared for each probe of the present invention. A perfect mismatch probe has the same sequence as the original probe (i.e., the perfect match probe) except for a homomeric substitution (A to T, T to A, G to C, and C to G) at or near the center of the perfect mismatch probe. For instance, if the original probe has 2n nucleotide residues, the homomeric substitution in the perfect mismatch probe is either at the n or n+1 position, but not at both positions. If the original probe has 2n+1 nucleotide residues, the homomeric substitution in the perfect mismatch probe is at the n+1 position.

The polynucleotide probes of the present invention can be synthesized using a variety of methods. Examples of these methods include, but are not limited to, the use of automated or high throughput DNA synthesizers, such as those provided by Millipore, GeneMachines, and BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or any combination thereof.

The parent sequences, tiling sequences, and polynucleotide probes of the present invention can be used to detect, identify, distinguish, or quantitate different Staphylococcus aureus strains in a sample of interest. Suitable methods for this purpose include, but are not limited to, nucleic acid arrays (including bead arrays), Southern Blot, Northern Blot, PCR, and RT-PCR. A sample of interest can be, without limitation, a food sample, an environmental sample, a pharmaceutical sample, a clinical sample, a blood sample, a body fluid sample, a waste sample, a human or animal sample, a bacterial culture, or any other biological or chemical sample.

As appreciated by those skilled in the art, parent sequences can be similarly isolated from the genomic sequences of other non-viral species. These parent sequences include ORFs or other transcribable elements. Tiling sequences and polynucleotide probes can be prepared from these parent sequences using the methods described above.

### C. NUCLEIC ACID ARRAYS

The polynucleotide probes of the present invention can be used to make nucleic acid arrays for the concurrent or discriminable detection of different strains of Staphylococcus aureus or other non-viral species. In many embodiments, the nucleic acid arrays of the present invention include at least one substrate support which has a plurality of discrete regions. The location of each of these discrete regions is either known or determinable. The discrete regions can be organized in various forms or patterns. For instance, the discrete regions can be arranged as an array of regularly spaced areas on a surface of the substrate. Other regular or irregular patterns, such as linear, concentric or spiral patterns, can be used.

Polynucleotide probes can be stably attached to respective discrete regions through covalent or non-covalent interactions. As used herein, a polynucleotide probe is “stably” attached to a discrete region if the polynucleotide probe retains its position relative to the discrete region during nucleic acid array hybridization.

Any method may be used to attach polynucleotide probes to a nucleic acid array of the present invention. In one embodiment, polynucleotide probes are covalently attached to a substrate support by first depositing the polynucleotide probes to respective discrete regions on a surface of the substrate support and then exposing the surface to a solution of a cross-linking agent, such as glutaraldehyde, borohydride, or other bifunctional agents. In another embodiment, polynucleotide probes are covalently bound to a substrate via an alkylamino-linker group or by coating a substrate (e.g., a glass slide) with polyethylenimine followed by activation with cyanuric chloride for coupling the polynucleotides. In yet another embodiment, polynucleotide probes are covalently attached to a nucleic acid array through polymer linkers. The polymer linkers may improve the accessibility of the probes to their purported targets. In many cases, the polymer linkers are not involved in the interactions between the probes and their purported targets.

Polynucleotide probes can also be stably attached to a nucleic acid array through non-covalent interactions. In one embodiment, polynucleotide probes are attached to a substrate support through electrostatic interactions between positively charged surface groups and the negatively charged probes. In another embodiment, a substrate employed in the present invention is a glass slide having a coating of a polycationic polymer on its surface, such as a cationic polypeptide. The polynucleotide probes are bound to these polycationic polymers. In yet another embodiment, the methods described in U.S. Pat. No. 6,440,723 are used to stably attach polynucleotide probes to a nucleic acid array of the present invention.

Numerous materials can be used to make the substrate supports) of a nucleic acid array of the present invention. Suitable materials include, but are not limited to, glass, silica, ceramics, nylon, quartz wafers, gels, metals, and paper. The substrate supports can be flexible or rigid. In one embodiment, they are in the form of a tape that is wound up on a reel or cassette. Two or more substrate supports can be used in the same nucleic acid array.

In many embodiments, the substrate supports are non-reactive with reagents that are used in nucleic acid array hybridization.

The surface(s) of a substrate support can be smooth and substantially planar. The surface(s) of the substrate can also have a variety of configurations, such as raised or depressed regions, trenches, v-grooves, mesa structures, or other regular or irregular configurations. The surface(s) of the substrate can be coated with one or more modification layers. Suitable modification layers include inorganic or organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on a nucleic acid array of the present invention can be of any size, shape and density. For instance, they can be squares, ellipsoids, rectangles, triangles, circles, or other regular or irregular geometric shapes, or any portion or combination thereof. In one embodiment, each of the discrete regions has a surface area of less than 10⁻¹ cm², such as less than 10⁻², 10⁻³, 10⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions may range, for example, between 50 and 50,000 regions/cm².

A variety of methods can be used to make the nucleic acid arrays of the present invention. For instance, the probes can be synthesized in a step-by-step manner on a substrate, or can be attached to a substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer (such as the DeskWriter C manufactured by Hewlett-Packard). In yet another embodiment, polynucleotide probes are immobilized on a nucleic acid array by using photolithography techniques.

The nucleic acid arrays of the present invention are capable of concurrently or discriminably detecting two or more different strains of a non-viral species, such as Staphylococcus aureus or other bacterial species. In one embodiment, a nucleic acid array of the present invention includes at least two polynucleotide probes, each of which is specific to a different strain of a non-viral species. Strain-specific probes can be prepared from the singleton sequences or other expressible sequences that are unique to that strain. In another embodiment, the nucleic acid array includes at least three, four, five, six, seven, eight, nine, ten, or more polynucleotide probes, each of which is specific to a different respective strain of a non-viral species.

In yet another embodiment, a nucleic acid array of the present invention includes at least one polynucleotide probe which is common to two or more different strains of a non-viral species. The common probe(s) can hybridize under stringent or nucleic acid array hybridization conditions to each and every strain selected from the two or more different strains. In still yet another embodiment, a nucleic acid array of the present invention includes at least one probe which is common to all of the different strains that are being investigated. This type of common probe can be derived from an ORF or a consensus sequence that is highly conserved among all of the different strains.

In a further embodiment, a nucleic acid array of the present invention includes two or more different polynucleotide probes that are specific to the same strain. For instance, a nucleic acid array can contain at least 5, 10, 20, 50, 100, 200 or more different probes, each of which is specific to the same strain. These different probes can hybridize under stringent or nucleic acid array hybridization conditions to the same RNA transcript, or different RNA transcripts of the same strain. They can be positioned in the same discrete region on a nucleic acid array. They can also be positioned in different discrete regions on a nucleic acid array.

In another embodiment, a nucleic acid array of the present invention can concurrently or discriminably detect two or more Staphylococcus aureus strains. Exemplary Staphylococcus aureus strains include, but are not limited to, COL, N315, Mu50, EMRSA-16, MSSA-476, MW2, and 8325. A nucleic acid array of the present invention can include at least two probes, each of which is specific to a different respective strain selected from the above Staphylococcus aureus strains. In one embodiment, a nucleic acid array of the present invention includes at least two, three, four, five, or six probes, each of which is specific to a different respective Staphylococcus aureus strain selected from COL, N315, Mu50, EMRSA-16, MSSA-476, and 8325.

In yet another embodiment, a nucleic acid array of the present invention contains at least one probe common to two or more Staphylococcus aureus strains selected from COL, N315, Mu50, EMRSA-16, MSSA-476, and 8325. In another embodiment, the common probe(s) can hybridize under stringent or nucleic acid array hybridization conditions to each and every strain selected from COL, N315, Mu50, EMRSA-16, MSSA-476, and 8325.

In still another embodiment, a nucleic acid array of the present invention includes polynucleotide probes which can hybridize under stringent or nucleic acid array hybridization conditions to respective sequences selected from SEQ ID NOs: 1 to 7,852, or the complements thereof. In one example, the nucleic acid array includes at least 2, 5, 10, 20, 30, 40, 50, 100, 200, 500, 1,000, 2,000, 3,000, 4,000, 5,000, or more different probes, each of which can hybridize under stringent or nucleic acid array hybridization conditions to a different respective sequence selected from SEQ ID NOs: 1 to 7,852, or the complement thereof. As used herein, two polynucleotides are “different” if they have different nucleic acid sequences.

In many embodiments, a nucleic acid array of the present invention includes two sets of probes. The first set of probes can hybridize under stringent or nucleic acid array hybridization conditions to respective sequences selected from SEQ ID NOs: 1 to 3,816, or the complements thereof, and the second set of probes can hybridize under the same conditions to respective sequences selected from SEQ ID NOs: 3,817 to 7,852, or the complements thereof. Each set can include at least 1, 2, 5, 10, 25, 50, 100, 200, 300, 400, 500, 1,000, or more probes.

In one embodiment, a nucleic acid array of the present invention includes probes for at least 1, 2, 5, 10, 50, 100, 500, 1,000, 2,000, 3,000, 4,000, 5,000, or more tiling sequences selected from SEQ ID NOs: 7,704. In another embodiment, a nucleic acid array of the present invention includes at least 2, 3, 4, 5, 10, 20, 30 or more probes for each tiling sequence of interest. In still another embodiment, the nucleic acid array includes probes for each tiling sequence selected from SEQ ID NOs: 7,704. Suitable probes for a tiling sequence include those depicted in SEQ ID NOs: 15,737.

The length of a probe can be selected to achieve the desired hybridization effect. For instance, a probe can include or consist of 15, 20, 25, 30, 35, 40, 45, 50, 60, 70, 80, 90, 100, 200, 300, 400 or more consecutive nucleotides. In one embodiment, each probe consists of about 25 consecutive nucleotides.

Multiple probes for the same gene can be included in a nucleic acid array of the present invention. For instance, at least 2, 5, 10, 15, 20, 25, 30 or more different probes can be used for detecting the same gene. Each of these different probes can be attached to a different respective region on a nucleic acid array. Alternatively, two or more different probes can be attached to the same discrete region. The concentration of one probe with respect to the other probe or probes in the same region may vary according to the objectives and requirements of the particular experiment. In one embodiment, different probes in the same region are present in approximately equimolar ratio.

In many applications, probes for different genes or RNA transcripts are attached to different respective regions on a nucleic acid array. In some other applications, probes for different genes or RNA transcripts are attached to the same discrete region.

In one embodiment, a nucleic acid array of the present invention is a bead array which includes a plurality of beads. Each bead is stably associated with one or more polynucleotide probes of the present invention.

In another embodiment, a nucleic acid array of the present invention includes probes for virulence or antimicrobial resistance genes. As used herein, a probe for a gene can hybridize under stringent or nucleic acid array hybridization conditions to an RNA transcript or a genomic sequence of that gene, or the complement thereof. In many instances, a probe for a gene is incapable of hybridizing under stringent or nucleic acid array hybridization conditions to RNA transcripts or genomic sequences of other genes, the complements thereof. The virulence or resistance genes that are being detected may be unique for a particular bacterial strain, or shared by several bacterial strains. Examples of virulence genes include, but are not limited to, various toxin and pathogenicity factor genes, such as those encoding fibrinogen binding protein, fibronectin binding protein, coagulase, enterotoxins, exotoxins, leukocidins, or V8 protease. Examples of antimicrobial resistance genes include, but are not limited to, penicillin-resistance genes, tetracycline-resistance genes, streptomycin-resistance genes, methicillin-resistance genes, and glycopeptide drug-resistance genes.

The nucleic acid arrays of the present invention can also include control probes which can hybridize under stringent or nucleic acid array hybridization conditions to respective control sequences, or the complements thereof. Examples of control sequences are depicted in SEQ ID NOs: 82,806. Table 3 lists the header information of each of these control sequences. Each header includes the identification number and other information of the corresponding control sequence. Example probes for these control sequences are described in Table G and SEQ ID NOs: 280,011.

TABLE 3
Control Sequences

| SEQ ID | Header |
|---|---|
| 82738 | >control:wyeSaureus2a:AFFX-BioB-3_at; gb\|J04423; J04423 E coli bioB gene |
| | biotin synthetase (-5, -M, -3 represent transcript regions 5 prime, Middle, and 3 |
| | prime respectively) |
| 82739 | >control:wyeSaureus2a:AFFX-BioB-5_at; gb\|J04423; J04423 E coli bioB gene |
| | biotin synthetase (-5, -M, -3 represent transcript regions 5 prime, Middle, and 3 |
| | prime respectively) |
| 82740 | >control:wyeSaureus2a:AFFX-BioB-M_at; gb\|J04423; J04423 E coli bioB gene |
| | biotin synthetase (-5, -M, -3 represent transcript regions 5 prime, Middle, and 3 |
| | prime respectively) |
| 82741 | >control:wyeSaureus2a:AFFX-BioC-3_at; gb\|J04423; J04423 E coli bioC protein |
| | (-5 and -3 represent transcript regions 5 prime and 3 prime respectively) |
| 82742 | >control:wyeSaureus2a:AFFX-BioC-5_at; gb\|J04423; J04423 E coli bioC protein |
| | (-5 and -3 represent transcript regions 5 prime and 3 prime respectively) |
| 82743 | >control:wyeSaureus2a:AFFX-BioDn-3_at; gb\|J04423; J04423 E coli bioD gene |
| | dethiobiotin synthetase (-5 and -3 represent transcript regions 5 prime and 3 |
| | prime respectively) |
| 82744 | >control:wyeSaureus2a:AFFX-BioDn-5_at; gb\|J04423; J04423 E coli bioD gene |
| | dethiobiotin synthetase (-5 and -3 represent transcript regions 5 prime and 3 |
| | prime respectively) |
| 82745 | >control:wyeSaureus2a:AFFX-CreX-3_at; gb\|X03453; X03453 Bacteriophage P1 |
| | cre recombinase protein (-5 and -3 represent transcript regions 5 prime and 3 |
| | prime respectively) |
| 82746 | >control:wyeSaureus2a:AFFX-CreX-5_at; gb\|X03453; X03453 Bacteriophage P1 |
| | cre recombinase protein (-5 and -3 represent transcript regions 5 prime and 3 |
| | prime respectively) |
| 82747 | >control:wyeSaureus2a:AFFX-DapX-3_at; gb\|L38424; L38424 B subtilis dapB, |
| | jojF, jojG genes corresponding to nucleotides 1358-3197 of L38424 (-5, -M, -3 |
| | represent transcript regions 5 prime, Middle, and 3 prime respectively) |
| 82748 | >control:wyeSaureus2a:AFFX-DapX-5_at; gb\|L38424; L38424 B subtilis dapB, |
| | jojF, jojG genes corresponding to nucleotides 1358-3197 of L38424 (-5, -M, -3 |
| | represent transcript regions 5 prime, Middle, and 3 prime respectively) |
| 82749 | >control:wyeSaureus2a:AFFX-DapX-M_at; gb\|L38424; L38424 B subtilis dapB, |
| | jojF, jojG genes corresponding to nucleotides 1358-3197 of L38424 (-5, -M, -3 |
| | represent transcript regions 5 prime, Middle, and 3 prime respectively) |
| 82750 | >control:wyeSaureus2a:AFFX-LysX-3_at; gb\|X17013; X17013 B subtilis lys |
| | gene for diaminopimelate decarboxylase corresponding to nucleotides 350-1345 |
| | of X17013 (-5, -M, -3 represent transcript regions 5 prime, Middle, and 3 prime |
| | respectively) |
| 82751 | >control:wyeSaureus2a:AFFX-LysX-5_at; gb\|X17013; X17013 B subtilis lys |
| | gene for diaminopimelate decarboxylase corresponding to nucleotides 350-1345 |
| | of X17013 (-5, -M, -3 represent transcript regions 5 prime, Middle, and 3 prime |
| | respectively) |
| 82752 | >control:wyeSaureus2a:AFFX-LysX-M_at; gb\|X17013; X17013 B subtilis lys |
| | gene for diaminopimelate decarboxylase corresponding to nucleotides 350-1345 |
| | of X17013 (-5, -M, -3 represent transcript regions 5 prime, Middle, and 3 prime |
| | respectively) |
| 82753 | >control:wyeSaureus2a:AFFX-PheX-3_at; gb\|M24537; M24537 B subtilis pheB, |
| | pheA genes corresponding to nucleotides 2017-3334 of |
| | M24537 (-5, -M, -3 represent transcript regions 5 prime, Middle, and 3 prime |
| | respectively) |
| 82754 | >control:wyeSaureus2a:AFFX-PheX-5_at; gb\|M24537; M24537 B subtilis pheB, |
| | pheA genes corresponding to nucleotides 2017-3334 of M24537 (-5, -M, -3 |
| | represent transcript regions 5 prime, Middle, and 3 prime respectively) |
| 82755 | >control:wyeSaureus2a:AFFX-PheX-M_at; gb\|M24537; M24537 B subtilis pheB, |
| | pheA genes corresponding to nucleotides 2017-3334 of M24537 (-5, -M, -3 |
| | represent transcript regions 5 prime, Middle, and 3 prime respectively) |
| 82756 | >control:wyeSaureus2a:AFFX-r2-Bs-dap-3_at; gb\|L38424; L38424 B subtilis |
| | dapB, jojF, jojG genes corresponding to nucleotides 1358-3197 of L38424 (-5, -M, |
| | -3 represent transcript regions 5 prime, Middle, and 3 prime respectively) |
| 82757 | >control:wyeSaureus2a:AFFX-r2-Bs-dap-5_at; gb\|L38424; L38424 B subtilis |
| | dapB, jojF, jojG genes corresponding to nucleotides 1358-3197 of L38424 (-5, -M, |
| | -3 represent transcript regions 5 prime, Middle, and 3 prime respectively) |
| 82758 | >control:wyeSaureus2a:AFFX-r2-Bs-dap-M_at; gb\|L38424; L38424 B subtilis |
| | dapB, jojF, jojG genes corresponding to nucleotides 1358-3197 of L38424 (-5, -M, |
| | -3 represent transcript regions 5 prime, Middle, and 3 prime respectively) |
| 82759 | >control:wyeSaureus2a:AFFX-r2-Bs-lys-3_at; gb\|X17013; X17013 B subtilis lys |
| | gene for diaminopimelate decarboxylase corresponding to nucleotides 350-1345 |
| | of X17013 (-5, -M, -3 represent transcript regions 5 prime, Middle, and 3 prime |
| | respectively) |
| 82760 | >control:wyeSaureus2a:AFFX-r2-Bs-lys-5_at; gb\|X17013; X17013 B subtilis lys |
| | gene for diaminopimelate decarboxylase corresponding to nucleotides 350-1345 |
| | of X17013 (-5, -M, -3 represent transcript regions 5 prime, Middle, and 3 prime |
| | respectively) |
| 82761 | >control:wyeSaureus2a:AFFX-r2-Bs-lys-M_at; gb\|X17013; X17013 B subtilis |
| | lys gene for diaminopimelate decarboxylase corresponding to nucleotides 350-1345 |
| | of X17013 (-5, -M, -3 represent transcript regions 5 prime, Middle, and 3 |
| | prime respectively) |
| 82762 | >control:wyeSaureus2a:AFFX-r2-Bs-phe-3_at; gb\|M24537; M24537 B subtilis |
| | pheB, pheA genes corresponding to nucleotides 2017-3334 of M24537 (-5, -M, -3 |
| | represent transcript regions 5 prime, Middle, and 3 prime respectively) |
| 82763 | >control:wyeSaureus2a:AFFX-r2-Bs-phe-5_at; gb\|M24537; M24537 B subtilis |
| | pheB, pheA genes corresponding to nucleotides 2017-3334 of M24537 (-5, -M, -3 |
| | represent transcript regions 5 prime, Middle, and 3 prime respectively) |
| 82764 | >control:wyeSaureus2a:AFFX-r2-Bs-phe-M_at; gb\|M24537; M24537 B subtilis |
| | pheB, pheA genes corresponding to nucleotides 2017-3334 of M24537 (-5, -M, -3 |
| | represent transcript regions 5 prime, Middle, and 3 prime respectively) |
| 82765 | >control:wyeSaureus2a:AFFX-r2-Bs-thr-3_s_at; gb\|X04603; Bacillus subtilis |
| | /REF = X04603 /DEF = B subtilis thrC, thrB genes corresponding to nucleotides |
| | 1689-2151 of X04603 /LEN = 2073 (-5, -M, -3 represent transcript regions 5 |
| | prime, Middle, and 3 prime respectively) |
| 82766 | >control:wyeSaureus2a:AFFX-r2-Bs-thr-5_s_at; gb\|X04603; Bacillus subtilis |
| | /REF = X04603 /DEF = B subtilis thrC, thrB genes corresponding to nucleotides |
| | 1689-2151 of X04603 /LEN = 2073 (-5, -M, -3 represent transcript regions 5 |
| | prime, Middle, and 3 prime respectively) |
| 82767 | >control:wyeSaureus2a:AFFX-r2-Bs-thr-M_s_at; gb\|X04603; Bacillus subtilis |
| | /REF = X04603 /DEF = B subtilis thrC, thrB genes corresponding to nucleotides |
| | 1689-2151 of X04603 /LEN = 2073 (-5, -M, -3 represent transcript regions 5 |
| | prime, Middle, and 3 prime respectively) |
| 82768 | >control:wyeSaureus2a:AFFX-r2-Ec-bioB-3_at; gb\|J04423; J04423 E coli bioB |
| | gene biotin synthetase (-5, -M, -3 represent transcript regions 5 prime, Middle, |
| | and 3 prime respectively) |
| 82769 | >control:wyeSaureus2a:AFFX-r2-Ec-bioB-5_at; gb\|J04423; J04423 E coli bioB |
| | gene biotin synthetase (-5, -M, -3 represent transcript regions 5 prime, Middle, |
| | and 3 prime respectively) |
| 82770 | >control:wyeSaureus2a:AFFX-r2-Ec-bioB-M_at; gb\|J04423; J04423 E coli bioB |
| | gene biotin synthetase (-5, -M, -3 represent transcript regions 5 prime, Middle, |
| | and 3 prime respectively) |
| 82771 | >control:wyeSaureus2a:AFFX-r2-Ec-bioC-3_at; gb\|J04423; J04423 E coli bioC |
| | protein (-5 and -3 represent transcript regions 5 prime and 3 prime respectively) |
| 82772 | >control:wyeSaureus2a:AFFX-r2-Ec-bioC-5_at; gb\|J04423; J04423 E coli bioC |
| | protein (-5 and -3 represent transcript regions 5 prime and 3 prime respectively) |
| 82773 | >control:wyeSaureus2a:AFFX-r2-Ec-bioD-3_at; gb\|J04423; J04423 E coli bioD |
| | gene dethiobiotin synthetase (-5 and -3 represent transcript regions 5 prime and 3 |
| | prime respectively) |
| 82774 | >control:wyeSaureus2a:AFFX-r2-Ec-bioD-5_at; gb\|J04423; J04423 E coli bioD |
| | gene dethiobiotin synthetase (-5 and -3 represent transcript regions 5 prime and 3 |
| | prime respectively) |
| 82775 | >control:wyeSaureus2a:AFFX-r2-P1-cre-3_at; gb\|X03453; X03453 |
| | Bacteriophage P1 cre recombinase protein (-5 and -3 represent transcript regions |
| | 5 prime and 3 prime respectively) |
| 82776 | >control:wyeSaureus2a:AFFX-r2-P1-cre-5_at; gb\|X03453; X03453 |
| | Bacteriophage P1 cre recombinase protein (-5 and -3 represent transcript regions |
| | 5 prime and 3 prime respectively) |
| 82777 | >control:wyeSaureus2a:AFFX-ThrX-3_at; gb\|X04603; X04603 B subtilis thrC, |
| | thrB genes corresponding to nucleotides 248-2229 of X04603 (-5, -M, -3 |
| | represent transcript regions 5 prime, Middle, and 3 prime respectively) |
| 82778 | >control:wyeSaureus2a:AFFX-ThrX-5_at; gb\|X04603; X04603 B subtilis thrC, |
| | thrB genes corresponding to nucleotides 248-2229 of X04603 (-5, -M, -3 |
| | represent transcript regions 5 prime, Middle, and 3 prime respectively) |
| 82779 | >control:wyeSaureus2a:AFFX-ThrX-M_at; gb\|X04603; X04603 B subtilis thrC, |
| | thrB genes corresponding to nucleotides 248-2229 of X04603 (-5, -M, -3 |
| | represent transcript regions 5 prime, Middle, and 3 prime respectively) |
| 82780 | >control:wyeSaureus2a:AFFX-TrpnX-3_at; gb\|K01391; K01391 B subtilis TrpE |
| | protein, TrpD protein, TrpC protein corresponding to nucleotides 1883-4400 of |
| | K01391 (-5, -M, -3 represent transcript regions 5 prime, Middle, and 3 prime |
| | respectively) |
| 82781 | >control:wyeSaureus2a:AFFX-TrpnX-5_at; gb\|K01391; K01391 B subtilis TrpE |
| | protein, TrpD protein, TrpC protein corresponding to nucleotides 1883-4400 of |
| | K01391 (-5, -M, -3 represent transcript regions 5 prime, Middle, and 3 prime |
| | respectively) |
| 82782 | >control:wyeSaureus2a:AFFX-TrpnX-M_at; gb\|K01391; K01391 B subtilis TrpE |
| | protein, TrpD protein, TrpC protein corresponding to nucleotides 1883-4400 of |
| | K01391 (-5, -M, -3 represent transcript regions 5 prime, Middle, and 3 prime |
| | respectively) |
| 82783 | >control:wyeSaureus2a:BIOB3_at; Unassigned; E. coli biotin synthetase (bioB), |
| | complete cds. |
| 82784 | >control:wyeSaureus2a:BIOB5_at; Unassigned; E. coli biotin synthetase (bioB), |
| | complete cds. |
| 82785 | >control:wyeSaureus2a:BIOBM_at; Unassigned; E. coli biotin synthetase (bioB), |
| | complete cds. |
| 82786 | >control:wyeSaureus2a:BIOC3_at; Unassigned; E. coli bioC protein, complete |
| | cds. |
| 82787 | >control:wyeSaureus2a:BIOC5_at; Unassigned; E. coli bioC protein, complete |
| | cds. |
| 82788 | >control:wyeSaureus2a:BIOD3_at; Unassigned; E. coli dethiobiotin synthetase |
| | (bioD), complete cds. |
| 82789 | >control:wyeSaureus2a:BIOD5_at; Unassigned; E. coli dethiobiotin synthetase |
| | (bioD), complete cds. |
| 82790 | >control:wyeSaureus2a:CRE3_at; Unassigned; Bacteriophage P1 cre gene for |
| | recombinase protein. |
| 82791 | >control:wyeSaureus2a:CRE5_at; Unassigned; Bacteriophage P1 cre gene for |
| | recombinase protein. |
| 82792 | >control:wyeSaureus2a:DAP3_at; Unassigned; Bacillus subtilis |
| | dihydropicolinate reductase (dapB), jojF, jojG, complete cds's. |
| 82793 | >control:wyeSaureus2a:DAP5_at; Unassigned; Bacillus subtilis |
| | dihydropicolinate reductase (dapB), jojF, jojG, complete cds's. |
| 82794 | >control:wyeSaureus2a:DAPM_at; Unassigned; Bacillus subtilis |
| | dihydropicolinate reductase (dapB), jojF, jojG, complete cds's. |
| 82795 | >control:wyeSaureus2a:LYSA3_at; Unassigned; Bacillus subtilis lys gene for |
| | diaminopimelate decarboxylase (EC 4.1.1.20). |
| 82796 | >control:wyeSaureus2a:LYSA5_at; Unassigned; Bacillus subtilis lys gene for |
| | diaminopimelate decarboxylase (EC 4.1.1.20). |
| 82797 | >control:wyeSaureus2a:LYSAM_at; Unassigned; Bacillus subtilis lys gene for |
| | diaminopimelate decarboxylase (EC 4.1.1.20). |
| 82798 | >control:wyeSaureus2a:PHE3_at; Unassigned; Bacillus subtillis phenylalanine |
| | biosynthesis associated protein (pheB), and monofunctional prephenate |
| | dehydratase (pheA) genes, complete cds. |
| 82799 | >control:wyeSaureus2a:PHE5_at; Unassigned; Bacillus subtillis phenylalanine |
| | biosynthesis associated protein (pheB), and monofunctional prephenate |
| | dehydratase (pheA) genes, complete cds. |
| 82800 | >control:wyeSaureus2a:PHEM_at; Unassigned; Bacillus subtillis phenylalanine |
| | biosynthesis associated protein (pheB), and monofunctional prephenate |
| | dehydratase (pheA) genes, complete cds. |
| 82801 | >control:wyeSaureus2a:THR3_at; Unassigned; B. subtilis thrB and thrC genes |
| | for homoserine kinase and threonine synthase (EC 2.7.1.39 and EC 4.2.99.2, |
| | respectively). |
| 82802 | >control:wyeSaureus2a:THR5_at; Unassigned; B. subtilis thrB and thrC genes |
| | for homoserine kinase and threonine synthase (EC 2.7.1.39 and EC 4.2.99.2, |
| | respectively). |
| 82803 | >control:wyeSaureus2a:THRM_at; Unassigned; B. subtilis thrB and thrC genes |
| | for homoserine kinase and threonine synthase (EC 2.7.1.39 and EC 4.2.99.2, |
| | respectively). |
| 82804 | >control:wyeSaureus2a:TRP3_at; Unassigned; B. subtilis tryptophan (trp) operon, |
| | complete cds. |
| 82805 | >control:wyeSaureus2a:TRP5_at; Unassigned; B. subtilis tryptophan (trp) operon, |
| | complete cds. |
| 82806 | >control:wyeSaureus2a:TRPM_at; Unassigned; B. subtilis tryptophan (trp) |
| | operon, complete cds. |

The nucleic acid arrays of the present invention can further include mismatch probes as controls. In many instances, the mismatch residue is located near the center of a probe such that the mismatch is more likely to destabilize the duplex with the target sequence under the hybridization conditions. In one embodiment, the mismatch probe is a perfect mismatch probe. Each polynucleotide probe and its corresponding perfect mismatch probe can be stably attached to different respective regions on a nucleic acid array of the present invention.

### D. APPLICATIONS

The nucleic acid arrays of the present invention can be used for concurrent or discriminable detection of different strains of a non-viral species, such as Staphylococcus aureus or other bacterial species. The nucleic acid arrays of the present invention can also be used for detecting the presence or absence of a non-viral species, independent of the particular strain that is being investigated. Moreover, the nucleic acid arrays of the present invention can be used to monitor gene expression patterns in Staphylococcus aureus or other non-viral species. In addition, the nucleic acid arrays of the present invention can be used to type unknown strains of Staphylococcus aureus or other clinically important non-viral species. Furthermore, probes for the intergenic sequences allow for the detection of unidentified ORFs or other expressible sequences. These intergenic probes are also useful for mapping transcription factor binding sites.

In one embodiment, a nucleic acid array of the present invention contains probes specific for different Staphylococcus aureus strains (such as COL, N315, Mu50, EMRSA-16, MSSA-476, and 8325), and can be used for discriminably detecting different clinical isolates. In another embodiment, a nucleic acid array of the present invention includes probes for strain N315 intergenic regions as well as probes for predicted open reading frames. This allows for the genetic analysis of Staphylococcus aureus DNA and RNA content, including analysis of cis-acting regulatory elements. Probes for the intergenic sequences of other Staphylococcus aureus strains can also be included in a nucleic acid array of the present invention. These probes may be specific to a particular Staphylococcus aureus strain, or common to two or more Staphylococcus aureus strains.

Protocols for performing nucleic acid array analysis are well known in the art. Exemplary protocols include those provided by Affymetrix in connection with the use of its GeneChip® arrays. Samples amenable to nucleic acid array analysis include biological samples prepared from human or animal tissues, such as pus, blood, urine, or other body fluid, tissue or waste samples. In addition, food, environmental, pharmaceutical or other types of samples can be similarly analyzed using the nucleic acid arrays of the present invention.

In one embodiment, bacteria or other microbes in a sample of interest are grown in culture before being analyzed by a nucleic acid array of the present invention. In another embodiment, an originally collected sample is directly analyzed without additional culturing. In many cases, the microbes that are being analyzed are pathogens that can cause human or animal diseases.

In many embodiments, the nucleic acid array analysis involves isolation of nucleic acid from a sample of interest, followed by hybridization of the isolated nucleic acid to a nucleic acid array of the present invention. The isolated nucleic acid can be RNA or DNA (e.g., genomic DNA). In one embodiment, the isolated RNA is amplified or labeled before being hybridized to a nucleic acid array of the present invention. Various methods are available for isolating or enriching RNA. These methods include, but are not limited to, RNeasy kits (provided by QIAGEN), MasterPure kits (provided by Epicentre Technologies), and TRIZOL (provided by Gibco BRL). The RNA isolation protocols provided by Affymetrix can also be employed in the present invention.

In another embodiment, bacterial mRNA is enriched by removing 16S and 25S rRNA. Different methods are available to eliminate or reduce the amount of rRNA in a bacterial sample. For instance, the MICROBExpress kit (provided by Ambion, Inc.) uses oligonucleotide-attached beads to capture and remove rRNA. 16S and 25S rRNA can also be removed by enzyme digestions. According to the latter method, 16S and 25S rRNA are first amplified using reverse transcriptase and specific primers to produce cDNA. The rRNA is allowed to anneal with the cDNA. The sample is then treated with RNAase H, which specifically digests RNA within an RNA:DNA hybrid.

In yet another embodiment, mRNA is amplified before being subject to nucleic acid array analysis. Suitable mRNA amplification methods include, but are not limited to, reverse transcriptase PCR, isothermal amplification, ligase chain reaction, hexamer priming, and Qbeta replicase methods. The amplification products can be either cDNA or cRNA.

Polynucleotides for hybridization to a nucleic acid array can be labeled with one or more labeling moieties to allow for detection of hybridized polynucleotide complexes. Example labeling moieties can include compositions that are detectable by spectroscopic, photochemical, biochemical, bioelectronic, immunochemical, electrical, optical or chemical means. Example labeling moieties include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like. In one embodiment, the enriched bacterial mRNA is labeled with biotin. The 5′ end of the enriched bacterial mRNA is first modified by T4 polynucleotide kinase with γ-S-ATP. Biotin is then conjugated to the 5′ end of the modified mRNA using methods known in the art.

Polynucleotides can be fragmented before being labeled with detectable moieties. Exemplary methods for fragmentation include, but are not limited to, heat or ion-mediated hydrolysis.

Hybridization reactions can be performed in absolute or differential hybridization formats. In the absolute hybridization format, polynucleotides derived from one sample are hybridized to the probes in a nucleic acid array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

Signals gathered from nucleic acid arrays can be analyzed using commercially available software, such as those provide by Affymetrix or Agilent Technologies. Controls, such as for scan sensitivity, probe labeling and cDNA or cRNA quantitation, may be included in the hybridization experiments. Examples of control sequences are listed in Table 3. The array hybridization signals can be scaled or normalized before being subject to further analysis. For instance, the hybridization signal for each probe can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes.

The present invention also features protein arrays for the concurrent or discriminable detection of multiple strains of a non-viral species. Each protein array of the present invention includes probes which can specifically bind to respective proteins of a non-viral species. In one embodiment, the probes on a protein array of the present invention are antibodies. Many of these antibodies can bind to the respective proteins with an affinity constant of at least 10⁴ M⁻¹, 10⁵ M⁻¹, 10⁻⁶ M⁻¹, 10⁷ M⁻¹, or more. In many instances, an antibody for a specified protein does not bind to other proteins. Suitable antibodies for the present invention include, but are not limited to, polyclonal antibodies, monoclonal antibodies, chimeric antibodies, single chain antibodies, Fab fragments, or fragments produced by a Fab expression library. Other peptides, scaffolds, or protein-binding ligands can also be used to construct the protein arrays of the present invention.

Numerous methods are available for immobilizing antibodies or other probes on a protein array of the present invention. Examples of these methods include, but are not limited to, diffusion (e.g., agarose or polyacrylamide gel), surface absorption (e.g., nitrocellulose or PVDF), covalent binding (e.g., silanes or aldehyde), or non-covalent affinity binding (e.g., biotin-streptavidin). Examples of protein array fabrication methods include, but are not limited to, ink-jetting, robotic contact printing, photolithography, or piezoelectric spotting. The method described in MacBeath and Schreiber, SCIENCE, 289: 1760-1763 (2000) can also be used. Suitable substrate supports for a protein array of the present invention include, but are not limited to, glass, membranes, mass spectrometer plates, microtiter wells, silica, or beads.

The protein-coding sequence of a gene can be determined by a variety of methods. For instance, many protein sequences can be obtained from the NCBI or other public or commercial sequence databases. The protein-coding sequences can also be extracted from the corresponding tiling or parent sequences by using an open reading frame (ORF) prediction program. Examples of ORF prediction programs include, but are not limited to, GeneMark (provided by the European Bioinformatics Institute), Glimmer (provided by TIGR), and ORF Finder (provided by the NCBI). Where a parent or tiling sequence represents the 5′ or 3′ untranslated region of a gene, a BLAST search of the sequence against a genome database can be conducted to determine the protein-coding region of the gene.

In one embodiment, a protein array of the present invention includes at least 2, 5, 10, 20, 30, 40, 50, 100, 200, 300, 400, 500, 1,000, 2,000, 3,000, 4,000, or more probes, each of which can specifically bind to a different respective protein encoded by SEQ ID NOs: 1-7,852 or their corresponding genes.

Furthermore, the present invention contemplates a collection of polynucleotides. A polynucleotide in the collection is capable of hybridizing under stringent or nucleic acid array hybridization conditions to a sequence selected from SEQ ID NOs: 1 to 7,852, or the complement thereof. In one embodiment, the collection includes two or more different polynucleotides, each of which is capable of hybridizing under stringent or nucleic acid array hybridization conditions to a different respective sequence selected from SEQ ID NOs: 1 to 7,852, or the complement thereof. In another embodiment, the collection includes one or more parent sequences depicted in SEQ ID NOs: 1 to 7,852, or one or more tiling sequences depicted in SEQ ID NOs: 7,704, or the complement(s) thereof. In still another embodiment, the collection includes one or more oligonucleotide probes listed in SEQ ID NOs: 15,737. In yet another embodiment, the polynucleotides in a collection of the present invention are stably attached to at least one substrate support to form a nucleic acid array. The present invention also features kits including the polynucleotides or polynucleotide probes of the present invention.

It should be understood that the above-described embodiments and the following examples are given by way of illustration, not limitation. Various changes and modifications within the scope of the present invention will become apparent to those skilled in the art from the present description.

### E. EXAMPLES

### Example 1

### Nucleic Acid Array

The tiling sequences depicted in SEQ ID NOs: 7,704 were submitted to Affymetrix for custom array design. Affymetrix selected probes for each tiling sequence using its probe-picking algorithm. Probes with 25 non-ambiguous bases were selected. A maximal set of 24-34 probes were selected for each submitted ORF sequence, and a maximal set of 12-15 probes were chosen for each submitted intergenic sequence. The final set of selected probes is depicted in SEQ ID NOs: 82,374. Table G shows the header for each of these probes. These probes are perfect match probes. The perfect mismatch probe for each perfect match probe was also prepared. The perfect mismatch probe is identical to the perfect match probe except at position 13 where a single-base substitution is made. The substitutions are A to T, T to A, G to C, or C to G. The final custom nucleic acid array includes both the perfect match probes and the perfect mismatch probes. In addition, the custom array contains probe sets for control sequences. The control probes are depicted in SEQ ID NOs: 279,085. The headers for the control sequences are also illustrated in Table G.

The nucleic acid array in this Example contains probes for at least 268 virulence gene loci, 46 resistance gene loci, 2,007 perfect ORFs (such as ribosomal proteins and DNA polymerase), 2,059 imperfect ORFs (including alleles with insertions, deletions or substitutions, splice variants, and strain-specific genes), and 3,343 intergenic regions. “Perfect ORFs” are ORF clusters that contain a representative sequence from each of the six genomes listed in Table 1. “Imperfect ORFs” refer to ORFs that are not present in all of the six input genomes listed in Table 1. The tiling or parent sequences for imperfect ORFs include, but are not limited to, AB009866-cds22_x_at, AB009866-cds25_at, AB009866-cds3_at, AB009866-cds50_x_at, AB009866-cds55_x_at, AB009866-cds56_at, AB033763-cds11_at, AB033763-cds2_at, AB033763-cds20_at, AB033763-cds27_at, AB033763-cds29_at, AB033763-cds4_at, AB033763-cds46_at, AB033763-cds5_at, AB033763-cds8_at, AB037671-cds10_at, AB037671-cds11_at, AB037671-cds21_at, AB037671-cds23_at, AB037671-cds28_at, AB037671-cds30_at, AB037671-cds32_at, AB037671-cds36_at, AB037671-cds46_at, AB037671-cds47_at, AB037671-cds49_at, AB037671-cds52_at, AB037671-cds53_at, AB037671-cds54_at, AB037671-cds55_at, AB037671-cds56_at, AB037671-cds57_at, AB037671-cds59_at, AB037671-cds6_at, AB037671-cds60_at, AB037671-cds61_at, AB037671-cds62_at, AB037671-cds63_at, AB037671-cds66_at, AB037671-cds67_at, AB037671-cds68_at, AB037671-cds69_at, AB037671-cds7_at, AB037671-cds70_at, AB037671-cds80_at, AB037671-cds81_at, AB037671-cds85_at, AB037671-cds87_at, AB047088-cds7_s_at, AB047089-cds1_at, AB047089-cds3_x_at, AB047089-cds4_at, AF051916-cds2_at, AF051917-cds10_at, AF051917-cds11_at, AF051917-cds12_at, AF051917-cds13_at, AF051917-cds14_at, AF051917-cds16_at, AF051917-cds36_at, AF051917-cds38_at, AF051917-cds7_at, AF051917-cds9_at, AF053140-cds2_at, AF077865-cds1_at, AF117258-cds1_at, AFF117258-cds2_at, AF117258-cds3_at, AF117259-cds1_at, AF117259-cds2_at, AF147744-cds1_at, AF147744-cds2_at, AF147744-cds3_at, AF147744-cds4_at, AF167161-cds1_at, AF167161-cds2_at, AF167161-cds7_at, AF186237-cds1_at, AF203376-cds1_at, AF203376-cds2_at, AF203377-cds1_at, AF203377-cds2_at, AF210055-cds1_at, AF217235-cds11_at, AF217235-cds18_at, AF217235-cds19_at, AF217235-cds20_at, AF217235-cds21_at, AF217235-cds5_at, AF217235-cds6_at, AF217235-cds8_x_at, AF217235-cds9_at, AF282215-cds2_at, AF282215-cds4_at, AF288402-cds1-seg1_at, AF288402-cds1-seg2_at, AJ005646-cds1_x_at, AJ243790-cds1_at, AJ277173-cds1_at, AJ292927-cds1_at, AJ309178-cds1_at, AJ309180-cds1_at, AJ309181-cds1_at, AJ309182-cds1_at, AJ309184-cds1_at, AJ309185-cds1_at, AJ309190-cds1_at, AJ309191-cds1_x_at, AJ311975-cds1_at, AJ311976-cds1_at, AJ311977-cds1_at, AP001553-cds10_at, AP001553-cds11_at, AP001553-cds12_at, AP001553-cds14_x_at, AP001553-cds2_at, AP001553-cds21_at, AP001553-cds27_at, AP001553-cds3_at, AP001553-cds30_at, AP001553-cds31_at, AP001553-cds37_x_at, AP001553-cds38_at, AP001553-cds39_at, AP001553-cds40_at, AP001553-cds41_at, AP001553-cds42_at, AP001553-cds43_at, AP001553-cds44_at, AP001553-cds45_at, AP001553-cds46_at, AP001553-cds47_at, AP001553-cds48_at, AP001553-cds49_at, AP001553-cds5_at, AP001553-cds50_at, AP001553-cds51_at, AP001553-cds52_at, AP001553-cds53_at, AP001553-cds54_at, AP001553-cds55_at, AP001553-cds56_at, AP001553-cds57_at, AP001553-cds6_at, AP001553-cds61_at, AP001553-cds64_at, AP001553-cds65_at, AP001553-cds8_at, AP001553-cds9_at, AY029184-cds1_at, D83951-cds2_at, J01763-cds1_at, J03947-cds1_at, L43082-cds1_at, M17348-cds1_at, M17990-cds1_at, M18086-cds1_s_at, M21319-cds1_at, M32470-cds1_at, M32470-cds2_at, M63917-cds1_at, U10927-cds1_at, U10927-cds10_at, U10927-cds11_at, U10927-cds12_at, U10927-cds13_at, U10927-cds2_at, U10927-cds3_at, U10927-cds4_at, U10927-cds5_at, U10927-cds6_at, U10927-cds7_at, U10927-cds8_at, U10927-cds9_at, U31979-cds4_at, U35036-cds4_at, U38429-cds3_at, U50077-cds2_x_at, U73025-cds1_at, U73026-cds1_at, U73027-cds1_at, U82085-cds1_at, U93688-cds1_x_at, U93688-cds10_at, U93688-cds12_at, U93688-cds15_at, U93688-cds8_at, U93688-cds9_at, U96610-cds1_s_at, WAN008YT9-seg1-x_at, WAN008YT9-seg2_x_at, WAN0144LN-seg1_s_at, WAN014A7L-5_at, WAN014A7L-M_at, WAN014A7M-seg1_x_at, WAN014A7M-seg2_at, WAN014A7N-seg1_at, WAN014A7N-seg2_at, WAN014A7O-seg1_at, WAN014A7O-seg2_at, WAN014A7P-seg1_at, WAN014A7P-seg2_at, WAN014A7Q-seg1_at, WAN014A7Q-seg2_at, WAN014A7R-seg1_at, WAN014A7R-seg2_s_at, WAN014A7S-5_at, WAN014A7S-M_at, WAN014A7T-5_at, WAN014A7T-M_at, WAN014A7U-3_at, WAN014A7U-M_at, WAN014A7V-5_at, WAN014A7V-M_at, WAN014A7W-5_at, U81980-cds2_at, WAN014A7W-M_at, WAN014A7X-5_at, WAN014A7X-M_at, WAN014A80-seg1_x_at, J04551-cds1_at, WAN014A7Y-seg1_at, WAN014A7Y-seg2_at, WAN014A7Z-seg1_x_at, WAN014A7Z-seg2_x_at, WAN014A80-seg2_x_at, WAN014A81-5_at, WAN014A81-M_at, WAN014A82-seg2_at, U19459-cds1_at, WAN014A83-5_at, WAN014A83-M_at, WAN014FR7_at, WAN014FR8_at, WAN014FRB_at, WAN014FRE_at, WAN014FRF_at, WAN014FRG_at, WAN014FRH_at, WAN014FRK_at, WAN014FRL_at, WAN014FRM_at, WAN014FRO_at, WAN014FRP_at, WAN014FRR_at, WAN014FRU_at, WAN014FRW_at, WAN014FRX_at, WAN014FRY_at, WAN014FRZ_at, WAN014FS0_at, WAN014FS3_at, WAN014FS4_at, WAN014FS5_at, WAN014FS6_at, WAN014FS9_at, WAN014FSB_at, WAN014FSC_at, WAN014FSD_at, WAN014FSE_at, WAN014FSI_at, WAN014FSJ_at, WAN014FSK_at, WAN014FSL_at, WAN014FSM_at, WAN014FSP_at, WAN014FSQ_at, WAN014FSR_at, WAN014FSZ_at, WAN014FT0_at, WAN014FT1_at, WAN014FT2_at, WAN014FT3_at, WAN014FT5_at, WAN014FT7_at, WAN014FTD_at, WAN014FTH_at, WAN014FTI_at, WAN014FTJ_at, WAN014FTK_at, WAN014FT0_at, WAN014FTR_at, WAN014FTT_at, WAN014FTV_at, WAN014FTX_at, WAN014FTY_at, WAN014FTZ_at, WAN014FU0_at, WAN014FU_at, WAN014FU2_at, WAN014FU3_at, WAN014FU6_at, WAN014FU9_at, WAN014FUA_at, WAN014FUB_at, WAN014FUC_at, WAN014FUF_at, WAN014FUI_at, WAN014FUJ_at, WAN014FUK_at, WAN014FUL_at, WAN014FUM_at, WAN014FUS_at, WAN014FUV_at, WAN014FV5_at, WAN014FVP_at, WAN014FWI_at, WAN014FW9_at, WAN014FWE_at, WAN014FWL_at, WAN014FWM_at, WAN014FWN_at, WAN014FW0_at, WAN014FWS_at, WAN014FWT_at, WAN014FWU_at, WAN014FWW_at, WAN014FWX_at, WAN014FWZ_at, WAN014FX0_at, WAN014FXF-5_at, WAN014FXF-M_at, WAN014FXG_at, WAN014FY1_at, WAN014FY2_at, WAN014FYA_at, WAN014FYB_at, WAN014FYC_at, WAN014FYH_at, WAN014FYP_at, WAN014FZ0_at, WAN014FZ5_at, WAN014FZE_at, WAN014FZI_at, WAN014FZK_at, WAN014FZM_at, WAN014FZN_at, WAN014FZ0_at, WAN014FZP_at, WAN014FZU_at, WAN014FZW_at, WAN014G09_at, WAN014G0A_at, WAN014G0B_at, WAN014G0E_at, WAN014G0F_at, WAN014G0H_at, WAN014G0I_at, WAN014G0J_at, WAN014G0O_at, WAN014G0Q_at, WAN014G0S_at, WAN014G0T_at, WAN014G12_at, WAN014G16_at, WAN014G17_at, WAN014G18_at, WAN014G19_at, WAN014G1A_at, WAN014G1B_at, WAN014G1C_at, WAN014G1D_at, WAN014G1F_at, WAN014G1G_at, WAN014G1H_at, WAN014G1I_at, WAN014G1J_at, WAN014G1K_at, WAN014G1L_at, WAN014G1M_at, WAN014G1N_at, WAN014G1O_at, WAN014G1R_s_at, WAN014G20_at, WAN014G21_at, WAN014G2A_at, WAN014G2B_at, WAN014G2E_at, WAN014G2F_at, WAN014G2H_at, WAN014G2N_at, WAN014G2P_at, WAN014G2Q_at, WAN014G32_at, WAN014G34_at, WAN014G35_at, WAN014G36_at, WAN014G37_s_at, WAN014G38_at, WAN014G39_at, WAN014G3B_at, WAN014G3I_at, WAN014G3J_x_at, WAN014G3L_at, WAN014G3M_at, WAN014G3N_at, WAN014G3O_at, WAN014G3Q_at, WAN014G3V_at, WAN014G3W_at, WAN014G3X_x_at, WAN014G43_at, WAN014G4C_at, WAN014G4D_at, WAN014G4E_at, WAN014G4F_at, WAN014G4G_x_at, WAN014G4H_at, WAN014G4K_at, WAN014G4L_at, WAN014G4O_at, WAN014G4P_at, WAN014G4S_at, WAN014G4U_at, WAN014G4V_at, WAN014G4W_at, WAN014G4Y_x_at, WAN014G51_at, WAN014G54_at, WAN014G57_at, WAN014G5F₁₃ at, WAN014G5G_at, WAN014G5I_at, WAN014G5K_at, WAN014G5M_at, WAN014G5O_at, WAN014G5Y_at, WAN014G61_at, WAN014G63_at, WAN014G66_at, WAN014G67_at, WAN014G6D_at, WAN014G6E_at, WAN014G6I_at, WAN014G6J_at, WAN014G6V_at, WAN014G6W_at, WAN014G6X_at, WAN014G6Y_at, WAN014G73_x_at, WAN014G74_x_at, WAN014G7H_at, WAN014G7L_at, WAN014G7P_at, WAN014G7Q_at, WAN014G7V_at, WAN014G7W_at, WAN014G7X_at, WAN014G7Y_at, WAN014G7Z_at, WAN014G84_at, WAN014G85_at, WAN014G87_at, WAN014G8A_at, WAN014G8I_at, WAN014G8O_at, WAN014G8R_at, WAN014G90_at, WAN014G9H_at, WAN014G9K_at, WAN014G9L_at, WAN014G9M_at, WAN014G9P_at, WAN014G9X_at, WAN014GA2_at, WAN014GA3_at, WAN014GA4_at, WAN014GA5_at, WAN014GA6_at, WAN014GA9_at, WAN014GAA_at, WAN014GAC_at, WAN014GAD_at, WAN014GAH_at, WAN014GAI_at, WAN014GAJ_at, WAN014GAN_at, WAN014GAQ_x_at, WAN014GAS_at, WAN014GAT_at, WAN014GAU_at, WAN014GAW_x_at, WAN014GAY_at, WAN014GAZ_x_at, WAN014 GB0_x_at, WAN014 GB1_at, WAN014 GB2_at, WAN014 GB3_at, WAN014 GB7_at, WAN014 GB8_at, WAN014 GBF_at, WAN014 GBL_at, WAN014 GBM_at, WAN014 GBU_at, WAN014GC2_at, WAN014GC4_at, WAN014GC9_at, WAN014GCB_at, WAN014GCJ_at, WAN014GCM_at, WAN014GCN_at, WAN014GCP_at, WAN014GCR_at, WAN014GCT_at, WAN014GCV_at, WAN014GCW_at, WAN014GCX_at, WAN014GD6_at, WAN014GDD_at, WAN014GDG_x_at, WAN014GDL_at, WAN014GDM_at, WAN014GDN_at, WAN014GDP_at, WAN014GDY_at, WAN014GDZ_at, WAN014GE4_at, WAN014GE6_at, WAN014GE8_at, WAN014GEA_at, WAN014GEB_at, WAN014GEC_x_at, WAN014GET_at, WAN014GEW_at, WAN014GEY_at, WAN014GF1_at, WAN014GF2_at, WAN014GF4_at, WAN014GF6_at, WAN014GF9_at, WAN014GFA_at, WAN014GFB_at, WAN014GFC_at, WAN014GFH_at, WAN014GFJ_at, WAN014GFK_at, WAN014GFN_at, WAN014GFO_at, WAN014GFP_at, WAN014GFS_at, WAN014GFT_at, WAN014GFU_x_at, WAN014GFV_at, WAN014GFW_at, WAN014GFY_at, WAN014GG1_at, WAN014GG2_at, WAN014GG3_at, WAN014GG4_at, WAN014GG5_at, WAN014GG8_at, WAN014GG9_at, WAN014GGA_at, WAN014GGB_at, WAN014GGC_at, WAN014GGE_at, WAN014GGH_at, WAN014GGJ_at, WAN014GGK_at, WAN014GGL_at, WAN014GGM_at, WAN014GGN_at, WAN014GGO_at, WAN014GGP_at, WAN014GGQ_at, WAN014GGR_at, WAN014GGS_at, WAN014GGT_at, WAN014GGU_at, WAN014GGV_at, WAN014GGW_at, WAN014GGX_x_at, WAN014GGY_x_at, WAN014GGZ_at, WAN014 GH1_at, WAN014 GH2_at, WAN014 GH3_at, WAN014 GH4_at, WAN014 GH6_at, WAN014 GH7_at, WAN014 GH8_at, WAN014 GHA_at, WAN014 GHB_at, WAN014 GHC_at, WAN014 GHD_at, WAN014 GHE_at, WAN014 GHH_at, WAN014 GHJ_x_at, WAN014 GHM_x_at, WAN014 GHN_at, WAN014 GHO_at, WAN014 GHR_at, WAN014 GHS_at, WAN014 GHU_at, WAN014 GHW_at, WAN014 GHZ_at, WAN014GI0_at, WAN014GI1_at, WAN014GI6_at, WAN014GI9_x_at, WAN014GIA_at, WAN014GIB_at, WAN014GID_at, WAN014GIF_at, WAN014GII_at, WAN014GIJ_at, WAN014GIK_at, WAN014GIL_at, WAN014GIM_at, WAN014GIN_at, WAN014GIO_x_at, WAN014GIR_at, WAN014GIS_at, WAN014GIT_at, WAN014GIY_at, WAN014GIZ_at, WAN014GJ0_at, WAN014GJ1_at, WAN014GJ2_at, WAN014GJ5_at, WAN014GJ6_at, WAN014GJ7_at, WAN014GJ8_at, WAN014GJC_at, WAN014GJD_at, WAN014GJF_at, WAN014GJG_at, WAN014GJH_at, WAN014GJJ_at, WAN014GJK_at, WAN014GJU_at, WAN014GJW_at, WAN014GJX_at, WAN014GKO_at, WAN014GK4_at, WAN014GK5_at, WAN014GK6_at, WAN014GK7_at, WAN014GKA_x_at, WAN014GKD_at, WAN014GKE_at, WAN014GKF_at, WAN014GKG_at, WAN014GKH_at, WAN014GKI_at, WAN014GKK_at, WAN014GKM_at, WAN014GKN_at, WAN014GKO_at, WAN014GKP_at, WAN014GKQ_at, WAN014GKU_x_at, WAN014GKW_at, WAN014GKY_at, WAN014GKZ_at, WAN014GL0_at, WAN014GL1_at, WAN014GL2_at, WAN014GL3_at, WAN014GL4_at, WAN014GL7_at, WAN014GL8_at, WAN014GL9_at, WAN014GLA_s_at, WAN014GLB_at, WAN014GLC_at, WAN014GLD_at, WAN014GLE_at, WAN014GLF_at, WAN014GLG_at, WAN014GLH_at, WAN014GLI_at, WAN014GLJ_at, WAN014GLK_at, WAN014GLL_at, WAN014GLM_at, WAN014GLO_at, WAN014GLP_at, WAN014GLQ_at, WAN014GLR_at, WAN014GLS_at, WAN014GLT_at, WAN014GLU_at, WAN014GLV_at, WAN014GLW_at, WAN014GLX_at, WAN014GLY_at, WAN014GLZ_at, WAN014GM2_at, WAN014GM6_at, WAN014GM7_at, WAN014GM8_at, WAN014GMB_at, WAN014GMC_at, WAN014GMD_at, WAN014GME_at, WAN014GMF_at, WAN014GMG_at, WAN014GMH_at, WAN014GMK_at, WAN014GML_at, WAN014GMM_at, WAN014GMN_at, WAN014GMQ_at, WAN014GMS_at, WAN014GMT_at, WAN014GMU_at, WAN014GMV_at, WAN014GMX_at, WAN014GMZ_at, WAN014GNO_at, WAN014GN1_at, WAN014GN2_at, WAN014GN4_at, WAN014GNC_at, WAN014GNK_at, WAN014GNM_at, WAN014GNN_at, WAN014GNP_at, WAN014GNT_at, WAN014GNV_at, WAN014GNX_at, WAN014GNY_at, WAN014GO0_at, WAN014GO3_x_at, WAN014GO4_x_at, WAN014GO6_at, WAN014GO8_at, WAN014GO9_x_at, WAN014GOA_at, WAN014GOB_at, WAN014GOD_at, WAN014GOF_at, WAN014GOG_at, WAN014GOI_at, WAN014GOK_at, WAN014GOL_at, WAN014GON_x_at, WAN014GOO_x_at, WAN014GOP_at, WAN014GOT_at, WAN014GOW_at, WAN014GOX_at, WAN014GOY_at, WAN014GOZ_at, WAN014GP2_at, WAN014GP9_at, WAN014GPB_at, WAN014GPD_at, WAN014GPE_at, WAN014GPF_at, WAN014GPH_at, WAN014GPL_at, WAN014GPS_at, WAN014GPT_at, WAN014GPV_at, WAN014GPX_at, WAN014GPY_at, WAN014GQ2_at, WAN014GQ4_at, WAN014GQ9_at, WAN014GQA_at, WAN014GQE_at, WAN014GQF_at, WAN014GQG_at, WAN014GQH_s_at, WAN014GQJ_at, WAN014GQK_at, WAN014GQL_at, WAN014GQM_at, WAN014GQN_at, WAN014GQO_at, WAN014GQP_at, WAN014GQQ_at, WAN014GQR_at, WAN014GQU_x_at, WAN014GQX_at, WAN014GQZ_at, WAN014GR3_at, WAN014GR5_at, WAN014GR9_at, WAN014GRC_at, WAN014GRF_at, WAN014GRG_s_at, WAN014GRI_at, WAN014GRM_at, WAN014GRN_at, WAN014GRW_at, WAN014GRY_at, WAN014GRZ_at, WAN014GS4_at, WAN014GS5_at, WAN014GS6_at, WAN014GSB_at, WAN014GSD_at, WAN014GSF_at, WAN014GSK_at, WAN014GSL_at, WAN014GSO_at, WAN014GSP_at, WAN014GSS_at, WAN014GST_at, WAN014GSU_at, WAN014GSV_at, WAN014GSW_at, WAN014GSZ_x_at, WAN014GT0_x_at, WAN014GT1_at, WAN014GT2_at, WAN014GT6_at, WAN014GT8_x_at, WAN014GTB_at, WAN014GTC_at, WAN014GTD_at, WAN014GTF_at, WAN014GTW_at, WAN014GTY_at, WAN014GUD_at, WAN014GUL_at, WAN014GUM_at, WAN014GUN_at, WAN014GUS_at, WAN014GUU_at, WAN014GUV_at, WAN014GUX_at, WAN014GV0_at, WAN014GV1_at, WAN014GV6_at, WAN014GV7_at, WAN014GVA_at, WAN014GVC_at, WAN014GVE_at, WAN014GVH_at, WAN014GVN_at, WAN014GVO_at, WAN014GVW_at, WAN014GW1_at, WAN014GW3_at, WAN014GW6_at, WAN014GW8_at, WAN014GW9_at, WAN014GWB_x_at, WAN014GWD_x_at, WAN014GWE_at, WAN014GWJ_at, WAN014GWK_at, WAN014GWM_at, WAN014GWN_at, WAN014GWP_at, WAN014GWT_s_at, WAN014GWW_at, WAN014GWY_at, WAN014GWZ_at, WAN014GX4_s_at, WAN014GX5_at, WAN014GX6_at, WAN014GXC_x_at, WAN014GXX_at, WAN014GY1_at, WAN014GY3_at, WAN014GY6_at, WAN014GY9_at, WAN014GYH_at, WAN014GYT_at, WAN014GYU_at, WAN014GZO_at, WAN014GZC_at, WAN014GZN_at, WAN014GZX_at, WAN014H0K_at, WAN014H0L_at, WAN014H16_at, WAN014H17_at, WAN014H1B_at, WAN014H1N_at, WAN014H1S_at, WAN014H2A_at, WAN014H2E_at, WAN014H2G_at, WAN014H2J_at, WAN014H2K_x_at, WAN014H2L_x_at, WAN014H2M_at, WAN014H2W_at, WAN014H36_at, WAN014H39_at, WAN014H3G_at, WAN014H3M_at, WAN014H4K_at, WAN014H40_at, WAN014H4Q_at, WAN014H4S_at, WAN014H4U_at, WAN014H4V_at, WAN014H4W_at, WAN014H4X_at, WAN014H4Y_at, WAN014H4Z_at, WAN014H50_at, WAN014H51_at, WAN014H52_at, WAN014H53_at, WAN014H54_at, WAN014H55_at, WAN014H56_at, WAN014H57_at, WAN014H5A_at, WAN014H₅B_at, WAN014H₅C_at, WAN014H5D_at, WAN014H5E_at, WAN014H₅F_at, WAN014H5G_at, WAN014H5I_at, WAN014H5K_at, WAN014H5M_at, WAN014H5U_at, WAN014H67_at, WAN014H6K_at, WAN014H6R_at, WAN014H6U_at, WAN014H6X_at, WAN014H71_at, WAN014H74_at, WAN014H77_s_at, WAN014H₇B_at, WAN014H70_at, WAN014H7_at, WAN014H8P_at, WAN014H96_at, WAN014H9E_at, WAN014H₉F_at, WAN014H9H_at, WAN014HAT_at, WAN014HAU_at, WAN014HB8_at, WAN014HBN_at, WAN014HBP_at, WAN014HBS_at, WAN014HC8_at, WAN014HC9_at, WAN014HCB_at, WAN014HCK_at, WAN014HCS_at, WAN014HD0_at, WAN014HDK_at, WAN014HEC_at, WAN014HEI_at, WAN014HEK_at, WAN014HEL_at, WAN014HEW_at, WAN014HF9_at, WAN014HFJ_at, WAN014HFM_at, WAN014HFO_at, WAN014HFP_at, WAN014HFQ_at, WAN014HFR_at, WAN014HFS_at, WAN014HFU_at, WAN014HFV_at, WAN014HFW_at, WAN014HFX_at, WAN014HFZ_at, WAN014HG0_at, WAN014HG1_at, WAN014HG2_at, WAN014HG4_x_at, WAN014HG5_at, WAN014HG9_at, WAN014HGA_x_at, WAN014HGB_x_at, WAN014HGC_at, WAN014HGD_x_at, WAN014HGF_x_at, WAN014HGI_at, WAN014HGJ_at, WAN014HGK_at, WAN014HGL_at, WAN014HGN_at, WAN014HGQ_at, WAN014HGS_at, WAN014HGT_at, WAN014HGU_at, WAN014HGV_at, WAN014HGW_at, WAN014HGX_at, WAN014HGZ_at, WAN014HH1_at, WAN014HH2_at, WAN014HH5_at, WAN014HH7_at, WAN014HHA_at, WAN014HHB_at, WAN014HHC_at, WAN014HHF_at, WAN014HHG_at, WAN014HHH_at, WAN014HHI_at, WAN014HHJ_at, WAN014HHM_at, WAN014HHN_at, WAN014HHQ1 at, WAN014HHR_at, WAN014HHS_at, WAN014HHT_at, WAN014HHU_at, WAN014HHV_at, WAN014HHY_at, WAN014HI1_at, WAN014HI2_at, WAN014HI3_at, WAN014HI5_at, WAN014HI6_at, WAN014HI7_at, WAN014HI8_at, WAN014HI9_at, WAN014HIA_at, WAN014HIB_at, WAN014HIC_at, WAN014HID_at, WAN014HIE_at, WAN014HII_at, WAN014HIJ_at, WAN014HIK_at, WAN014HIL_at, WAN014HIN_at, WAN014HIO_at, WAN014HIQ_at, WAN014HIR_at, WAN014HIS_at, WAN014HIT_at, WAN014HIV_at, WAN014HIW_at, WAN014HIX_at, WAN014HIY_at, WAN014HJ1₁₃ at, WAN014HJ2_at, WAN014HJ3_at, WAN014HJ6_at, WAN014HJB_at, WAN014HJC_at, WAN014HJF_at, WAN014HJG_at, WAN014HJJ_at, WAN014HJM_at, WAN014HJR_at, WAN014HJU_at, WAN014HK2_at, WAN014HK3_at, WAN014HK4_at, WAN014HK5_at, WAN014HKA_at, WAN014HKB_at, WAN014HKG_s_at, WAN014HKK_at, WAN014HKN_at, WAN014HKP_at, WAN014HKQ_at, WAN014HKT_at, WAN014HKY_at, WAN014HL0_at, WAN014HL1_at, WAN014HL5_at, WAN014HL6_at, WAN014HL8_at, WAN014HL9_at, WAN014HLA_at, WAN014HLB_at, WAN014HLC_at, WAN014HLD_at, WAN014HLF_at, WAN014HLG_at, WAN014HLH_at, WAN014HLM_at, WAN014HLN_at, WAN014HLO_at, WAN014HLQ_at, WAN014HLR_at, WAN014HLS_at, WAN014HLT_at, WAN014HLV_at, WAN014HLW_at, WAN014HLX_at, WAN014HLY_at, WAN014HLZ_at, WAN014HM1_at, WAN014HM2_at, WAN014HM4_at, WAN014HM6_at, WAN014HM7_at, WAN014HM9_at, WAN014HMA_at, WAN014HMC_at, WAN014HMD_at, WAN014HME_at, WAN014HMG_at, WAN014HMH_at, WAN014HMI_s_at, WAN014HMJ_at, WAN014HML_at, WAN014HMM_at, WAN014HMQ_at, WAN014HMR_at, WAN014HMS_at, WAN014HMT_at, WAN014HMW_at, WAN014HN4_at, WAN014HN5_at, WAN014HN6_at, WAN014HN8_at, WAN014HNB_at, WAN014HNG_at, WAN014HNK_at, WAN014HNL_at, WAN014HNM_at, WAN014HNO_x_at, WAN014HNP_at, WAN0144HNQ_at, WAN014HNT_at, WAN014HNU_at, WAN014HNV_at, WAN014HNW_at, WAN014HNX_at, WAN014HO0_at, WAN014HOC_at, WAN014HOD_at, WAN014HOG_at, WAN014HOH_at, WAN014HOI_at, WAN014HOJ_x_at, WAN014HOR_at, WAN014HOS_at, WAN014HOT_at, WAN014HOU_at, WAN014HOX_at, WAN014HOY_at, WAN014HOZ_at, WAN014HP0_at, WAN014HP2_at, WAN014HP3_at, WAN014HP4_at, WAN014HP6_at, WAN014HP7_at, WAN014HP8_at, WAN014HPA_at, WAN014HPO_at, WAN014HPS_at, WAN014HPV_at, WAN014HPW_at, WAN014HPX_at, WAN014HPY_at, WAN014HPZ_at, WAN014HQQ_at, WAN014HQ3_at, WAN014HQ4_at, WAN014HQ5_at, WAN014HQ6_at, WAN014HQ7_at, WAN014HQ9_at, WAN014HQD_at, WAN014HQI_at, WAN014HQN_at, WAN014HQP_at, WAN014HQQ_at, WAN014HQR_at, WAN014HQS_at, WAN014HQV_at, WAN014HQY_at, WAN014HQZ_at, WAN014HRO_at, WAN014HR5_x_at, WAN014HR7_at, WAN014HRA_at, WAN014HRG_at, WAN014HRJ_at, WAN014HRM_at, WAN014HRP_at, WAN014HRQ_at, WAN014HRR_at, WAN014HRS_at, WAN014HRX_at, WAN014HS8_at, WAN014HSF_at, WAN014HSK_at, WAN014HSL_at, WAN014HSM_at, WAN014HSN_at, WAN014HSO_at, WAN014HSP_at, WAN014HSQ_at, WAN014HST_at, WAN014HSU_at, WAN014HSV_at, WAN014HSW_at, WAN014HSX_at, WAN014HT0_at, WAN014HT1_at, WAN014HT2_at, WAN014HT8_at, WAN014HTI_at, WAN014HTN_at, WAN014HTW_at, WAN014HTX_at, WAN014HTY_at, WAN014HTZ_at, WAN014HU0_at, WAN014HU5_at, WAN014HU6_at, WAN014HU7_at, WAN014HU8_at, WAN014HUG_at, WAN014HUM_at, WAN014HUN_at, WAN014HUP_at, WAN014HUR_at, WAN014HUS_at, WAN014HUT_at, WAN014HUU_at, WAN014HUV_at, WAN014HUX_at, WAN014HUY_at, WAN014HUZ_at, WAN014HV0_at, WAN014HV1_at, WAN014HV2_at, WAN014HV3_at, WAN014HV4_at, WAN014HV5_at, WAN014HV7_at, WAN014HV8_at, WAN014HV9_at, WAN014HVB_at, WAN014HVC_at, WAN014HVF_at, WAN014HVG_at, WAN014HVJ_at, WAN014HVM_at, WAN014HVN_at, WAN014HVW_at, WAN014HW1_at, WAN014HW6_at, WAN014HW9_at, WAN014HWA_at, WAN014HWD_at, WAN014HWE_at, WAN014HWF_at, WAN014HWK_at, WAN014HWO_at, WAN014HWZ_at, WAN014HX2_at, WAN014HX3_at, WAN014HX4_at, WAN014HX5_at, WAN014HX9_at, WAN014HXE_at, WAN014HXG_at, WAN014HXH_at, WAN014HXI_at, WAN014HXJ_at, WAN014HXK_at, WAN014HXL_at, WAN014HXM_at, WAN014HXO_at, WAN014HXP_at, WAN014HXR_at, WAN014HXS_at, WAN014HXU_at, WAN014HXW_at, WAN014HXX_at, WAN014HY1_at, WAN014HY4_at, WAN014HYB_at, WAN014HYH_at, WAN014HYI_at, WAN014HYK_x_at, WAN014HYR_at, WAN014HYS_at, WAN014HYV_at, WAN014HYX_at, WAN014 HZ0_at, WAN014 HZ3_at, WAN014 HZ6_at, WAN014 HZ7_at, WAN014 HZ8_at, WAN014 HZ9_at, WAN014 HZK_at, WAN014 HZL_at, WAN014 HZM_at, WAN014 HZP_s_at, WAN014I06_at, WAN14I0D_at, WAN014I0Q_at, WAN014I0Z_at, WAN014I13_at, WAN014I14_at, WAN014I1Z_at, WAN014I21_at, WAN014I24_at, WAN014I2E_at, WAN014I2G_at, WAN014I21_at, WAN014I2K_at, WAN014I2L_at, WAN014I2M_at, WAN014I2R_at, WAN014I2T_at, WAN014I3B_at, WAN014I3E_at, WAN014I3G_at, WAN014I3K_at, WAN014I4O_at, WAN014I41_at, WAN014I4E_at, WAN014I4K_at, WAN04I4L_at, WAN014I4N_at, WAN014I4O_at, WAN014I4Q_at, WAN014I4V_at, WAN014I4W_at, WAN014I4X_at, WAN014I4Z_at, WAN014I52_at, WAN014I54_at, WAN014I56_at, WAN014I59_at, WAN014I5T_at, WAN014I6E_at, WAN014I6M_at, WAN014I6P_at, WAN014I6Q_at, WAN014I6U_at, WAN01416Z_x_at, WAN014I7K-seg1_at, WAN014I7K-seg2_at, WAN014I7K-seg3_at, WAN014I7K-seg4_at, WAN014I7K-seg5_x_at, WAN014IKC_x_at, WAN014IKD_x_at, WAN014IKE_x_at, WAN014IKF_x_at, WAN014IKG_x_at, WAN014IKH_x_at, WAN014IKI_at, WAN014IKJ_at, WAN014IKK_x_at, WAN014IKL_x_at, WAN014IKM_x_at, WAN014IKN_at, WAN014IKO_at, WAN014IKP_at, WAN014IKQ_at, WAN014IKR_at, WAN014IKS_at, WAN014IKT_at, WAN014IKU_at, WAN014IKV_at, WAN014IKW_at, WAN014IKX_at, WAN014IKY_x_at, WAN014IKZ_x_at, WAN014IL0_x_at, WAN014IL1_at, WAN014IL2_x_at, WAN014IL3_at, WAN014IL6_x_at, WAN014IL7_at, WAN014IL8_at, WAN014IL9_at, WAN014ILA_at, WAN014ILB_at, WAN014ILC_x_at, WAN014ILD_x_at, WAN014ILF_at, WAN014ILG_at, WAN014ILH_at, WAN014ILI_x_at, WAN014ILJ_at, WAN014ILK_at, WAN014ILL_x_at, WAN014ILM_at, WAN014ILO_x_at, WAN014ILT_x_at, WAN014ILU_x_at, WAN014ILV_at, WAN014ILW_x_at, WAN014ILX_at, WAN014ILY_x_at, WAN014ILZ_at, WAN014IM0_x_at, WAN014IM1_x_at, WAN014IM2_x_at, WAN014IM3_x_at, WAN014IM4_x_at, WAN014IM5_x_at, WAN014IM7_x_at, WAN014IM8_x_at, WAN014IMA_x_at, WAN014IMB_at, WAN014IMC_x_at, WAN014IMD_x_at, WAN014IME_x_at, WAN014IMF_x_at, WAN014IMH_x_at, WAN014IMI_at, WAN014IMJ_at, WAN014IMK_at, WAN014IML_at, WAN014IMM_at, WAN014IMN_at, WAN014IMO_x_at, WAN014IMP_at, WAN014IMQ_x_at, WAN014IMR_x_at, WAN014IMS_at, WAN014IMT_x_at, WAN014IMU_at, WAN014IMV_at, WAN014IMW_at, WAN014IMX_at, WAN014IMY_at, WAN014IMZ_at, WAN014IN0_at, WAN014IN1_x_at, WAN014IN2_at, WAN014IN6_x_at, WAN014IN7_x_at, WAN014IN9_at, WAN014INB_x_at, WAN014INC_x_at, WAN014INE_at, WAN014ING_x_at, WAN014INH_at, WAN014INI_at, WAN014INJ_at, WAN014INK_at, WAN014INO_at, WAN014INP_at, WAN014INQ_at, WAN014INR_x_at, WAN014INS_at, WAN014INT_x_at, WAN014INU_x_at, WAN014INV_at, WAN014INW_x_at, WAN014INX_at, WAN014INY_at, WAN014INZ_at, WAN014IO0_at, WAN014IO1_at, WAN014IO2_at, WAN014IO3_at, WAN014IO4_at, WAN014IO5_at, WAN014IO6_at, WAN014IO7_at, WAN014IO8_at, WAN014IO9_at, WAN014IOA_at, WAN014IOB_x_at, WAN014IOC_at, WAN014IOD_at, WAN014IOE_at, WAN014IOF_at, WAN014IOH_x_at, WAN014IOI_at, WAN014IOJ_at, WAN014IOK_at, WAN014IOL_at, WAN014IOM_at, WAN014ION_at, WAN014IOO_at, WAN014IOP_x_at, WAN014IOQ_x_at, WAN014IOR_at, WAN014IOS_at, WAN014IOV-seg1_at, WAN014IOW-seg2_at, WAN014IOX-seg3_at, WAN014IOY-seg4_x_at, WAN014IOZ-seg1_x_at, WAN014IP0-seg2_x_at, WAN014IP1-seg3_x_at, WAN014IP2_at, WAN014IP3_at, WAN014IP5_at, WAN014IP6_at, WAN014IP7_at, WAN014IP8_x_at, WAN014IP9_at, WAN014IPA_at, WAN014IPB_s_at, WAN014IPC_at, WAN014IPD_at, WAN014IPE_at, WAN014IPF_at, WAN014IPG_at, WAN014IPH_at, WAN014IPI_at, WAN014IPJ_at, WAN014IPK_at, WAN014IPL_at, WAN014IPM_at, WAN014IPN_at, WAN014IPO_at, WAN014IPP_x_at, WAN014IPQ_x_at, WAN014IPR_at, WAN014IPS_x_at, WAN014IPT_at, WAN014IPU_at, WAN014IPV_at, WAN014IPW_at, WAN014IPX_at, WAN014IPY_at, WAN014IPZ_at, WAN014IQ0_at, WAN014IQ1_at, WAN014IQ2_at, WAN014IQ3_at, WAN014IQ4_at, WAN014IQ5 at, WAN014IQ6_at, WAN014IQ7_at, WAN014IQ8_at, WAN014IQ9_at, WAN014IQA_at, WAN014IQB_at, WAN014IQC_at, WAN014IQD_at, WAN014IQE_at, WAN014IQF_at, WAN014IQG_at, WAN014IQH_at, WAN014IQI_at, WAN014IQJ_at, WAN014IQK_at, WAN014IQL_at, WAN014IQM_at, WAN014IQN_at, WAN014IQO_at, WAN014IQP_at, WAN014IQQ_at, WAN014IQR_at, WAN014IQS_at, WAN014IQT_at, WAN014IQU_at, WAN014IQV_at, WAN014IQW_at, WAN014IQX_at, WAN014IQY_at, WAN014IQZ_at, WAN014IR0_at, WAN014IR1_x_at, WAN014IR2_at, WAN014IR3_at, WAN014IR4_at, WAN014IR5_at, WAN014IR6_at, WAN014IR7_at, WAN014IR8_at, WAN014IR9_at, WAN014IRA_at, WAN014IRB_at, WAN014IRC_at, WAN014IRD_at, WAN014IRE_at, WAN014IRF_at, WAN014IRG_at, WAN014IRH_at, WAN014IRI_at, WAN014IRJ_at, WAN014IRK_at, WAN014IRL_at, WAN014IRM_at, WAN014IRN_at, WAN014IRO_at, WAN014IRP_at, WAN014IRQ_at, WAN014IRR_at, WAN014IRS_at, WAN014IRT_at, WAN014IRU_at, WAN014IRV_at, WAN014IRW_at, WAN014IRX_at, WAN014IRY_at, WAN014IRZ_at, WAN014ISO_at, WAN014IS1_at, WAN014IS2_at, WAN014IS3_at, WAN014IS4_at, WAN014IS5_at, WAN014IS6_at, WAN014IS7_at, WAN014IS8_at, WAN014IS9_at, WAN014ISA_at, WAN014ISB_at, WAN014ISC_at, WAN014ISD_at, WAN014ISE_at, WAN014ISF_at, WAN014ISG_at, WAN014ISH_at, WAN014ISI_at, WAN014ISJ_at, WAN014ISK_at, WAN014ISL_at, WAN014ISM_at, WAN014ISN_at, WAN014ISO_at, WAN014ISP_at, WAN014ISQ at, WAN014ISR-seg2_at, WAN014ISS-seg1_at, WAN014IST_at, WAN014ISU_at, WAN014ISV_at, WAN014ISW_at, WAN014ISX_at, WAN014ISY_at, WAN014ISZ_at, WAN014ITO_at, WAN014IT1_at, WAN014IT2_at, WAN014IT3_at, WAN014IT4_at, WAN014IT5_at, WAN014IT6_at, WAN014IT7_at, WAN014IT8_at, WAN014IT9_at, WAN014ITA_at, WAN014ITB_at, WAN014ITC_at, WAN014ITD_at, WAN014ITE_at, WAN014ITF_at, WAN014ITG_at, WAN014ITH_at, WAN014ITI_at, WAN014ITJ_at, WAN014ITK_at, WAN014ITL_at, WAN014ITM_at, WAN014ITN_at, WAN014ITO_s_at, WAN014ITP_s_at, WAN014ITQx_at, WAN014ITR_x_at, WAN014ITS_at, WAN014ITT_at, WAN014ITU_at, WAN014ITV_at, WAN014ITW_at, WAN014ITX_at, WAN014ITY_at, WAN014ITZ_at, WAN014IU0_at, WAN014IU1_at, WAN014IU2_at, WAN014IU3_at, WAN014IU4_at, WAN014IU5_at, WAN014IU6_at, WAN014IU7_at, WAN014IU8_at, WAN014IU9_at, WAN014IUA_at, WAN014IUB_at, WAN014IUC_at, WAN014IUD_at, WAN014IUE_at, WAN014IUF_at, WAN014IUG_at, WAN014IUH_at, WAN014IUI_at, WAN014IUJ_at, WAN014IUK_at, WAN014IUL_at, WAN014IUM_at, WAN014IUN_at, WAN014IUO_at, WAN014IUP_at, WAN014IUQ_at, WAN014IUR_at, WAN014IUS_at, WAN014IUT_at, WAN014IUU_at, WAN014IUV_at, WAN014IUW_at, WAN014IUX_at, WAN014IUY_at, WAN014IUZ_at, WAN014IV0_at, WAN014IV1_at, WAN014IV2_at, WAN014IV3_at, WAN014IV4 at, WAN014IV5_at, WAN014IV6 at, WAN014IV7_at, WAN014IV8_at, WAN014IV9_at, WAN014IVA_at, WAN014IVB_at, WAN014IVC_at, WAN014IVD_at, WAN014IVE_at, WAN014IVF_at, WAN014IVG_at, WAN014IVH_at, WAN014IVI_at, WAN014IVJ_at, WAN014IVK_at, WAN014IVL_at, WAN014IVM_at, WAN014IVN_at, WAN014IVO_at, WAN014IVP_at, WAN014IVQ_at, WAN014IVR_at, WAN014IVS_at, WAN014IVT_at, WAN014IVU_at, WAN014IVV_at, WAN014IVW_at, WAN014IVX_at, WAN014IVY_at, WAN014IVZ_at, WAN014IW0_at, WAN014IW1_at, WAN014IW2_at, WAN014IW3_at, WAN014IW4_at, WAN014IW5_at, WAN014IW6_at, WAN014IW7_at, WAN014IW8_at, WAN014IW9_at, WAN014IWA_at, WAN014IWB_at, WAN014IWC_at, WAN014IWD_at, WAN014IWE_at, WAN014IWF_at, WAN014IWG_at, WAN014IWH_at, WAN014IWI_at, WAN014IWJ_s_at, WAN014IWK_at, WAN014IWL_at, WAN014IWM_at, WAN014IWN_at, WAN014IWO_at, WAN014IWP_at, WAN014IWQ_at, WAN014IWR_at, WAN014IWS_at, WAN014IWT_at, WAN014IWU_at, WAN014IWV_at, WAN014IWW_at, WAN014IWX_at, WAN014IWY_at, WAN014IWZ_at, WAN014IX0_at, WAN014IX1_at, WAN014IX2_at, WAN014IX3_at, WAN014IX4_at, WAN014IX5_at, WAN014IX6_at, WAN014IX7_at, WAN014IX8_at, WAN014IX9_at, WAN014IXA_at, WAN014IXB_at, WAN014IXC_at, WAN014IXD_at, WAN014IXE_at, WAN014IXF_at, WAN014IXG_at, WAN014IXH_at, WAN014IXI_at, WAN014IXJ_s_at, WAN014IXK_at, WAN014IXL_at, WAN014IXM_x_at, WAN014IXN_s_at, WAN014IXO_at, WAN014XP_at, WAN014IXQ_at, WAN014IXR_s_at, WAN014IXS_at, WAN014IXT_at, WAN014IXU_at, WAN014IXV_at, WAN014IXW_at, WAN014IXX_at, WAN014IXY_at, WAN014IXZ_at, WAN014IY0_at, WAN014IY1_at, WAN014IY2_at, WAN014IY3_at, WAN014IY6_at, WAN014IY7_at, WAN014IY8_at, WAN014IY9_at, WAN014IYB_at, WAN014IYC_x_at, WAN014IYD_x_at, WAN014IYE_at, WAN014IYF_at, WAN014IYG_at, WAN014IYH_at, WAN014IYI_at, WAN01A4RB_at, WAN01A54A_sat, WAN01A54B_s_at, WAN01A54F_s_at, WAN01A54R_s_at, WAN01A551_x_at, WAN01A55C_at, WAN01A57W_x_at, WAN01A57X_x_at, WAN01A584_s_at, WAN01A5FE_x_at, WAN01A5HF_x_at, WAN01A5HH_x_at, WAN01A5HS_x_at, WAN01A6DL_at, WAN01A6G9_at, WAN01A6VJ_at, WAN01A72A_at, WAN01A72C_x_at, WAN01A72E_at, WAN01A72I_s_at, WAN01A72V_at, WAN01A72W_x_at, WAN01A72X_s_at, WAN01A732_s_at, WAN01A733_at, WAN01A736_at, WAN01A737_at, WAN01A738_x_at, WAN01A73B_at, WAN01A73E_x_at, WAN01A73K_x_at, WAN01A73Q_at, WAN01A73T_x_at, WAN01A76C_at, WAN01A771_x_at, WAN01A775_x_at, WAN01A776_at, WAN01A7HG_at, WAN01A7HH_at, WAN01A717_x_at, WAN01A719_x_at, WAN01A71H_at, WAN01A71J_x_at, WAN01A71M_x_at, WAN01A71N_x_at, WAN01A71Y_x_at, WAN01A7JA_at, WAN01A7QZ4x_at, WAN01A7RO_x_at, WAN01A7WT_at, WAN01A89R_at, WAN01A8BV_at, WAN01A8BX_s_at, WAN01A8MR_at, WAN01A8OD_at, WAN01A8SB_at, WAN01AATG_at, WAN01AAVO_at, WAN01AAVT_at, WAN01AAWB_s_at, WAN01AAZI_x_at, WAN01AAZK_at, WAN01ABOB_x_at, WAN01AB31_at, WAN01AB5H_at, WAN01ABG2_at, WAN01ABK6_at, WAN01ABL8_at, WAN01AC23_at, WAN01AC35_x_at, WAN01AC7M_at, WAN01ACHS_at, WAN01ACOT_at, WAN01ACQQ_at, WAN01BOTN_at, WAN01BOU7_at, WAN01BOU8_at, WAN01BOUE_x_at, WAN01BOUQ_at, WAN01BOVS_at, WAN01BOYW_at, WAN01BOZO_at, WAN01BOZB_at, WAN01BOZO_at, WAN01BP0A_at, WAN01BP10_x_at, WAN01BP1C_x_at, WAN01BP1M_at, WAN01BP23_at, WAN01BP26_at, WAN01BP4M_at, WAN01BP56_at, WAN01BP5E_at, WAN01BP62_at, WAN01BP6C_at, WAN01BP6R_at, WAN01BP71_at, WAN01BP7X_at, WAN01BPA1_x_at, WAN01BPBH_at, WAN01BPBU_at, WAN01BPDF_at, WAN01BPEU_x_at, WAN01BPFR_at, WAN01BPG8_x_at, WAN01BPH1_at, WAN01BPHD_at, WAN01BPJS_at, WAN01BPJZ_at, WAN01BPLH_at, WAN01BPNF_x_at, WAN01BPO9_at, WAN01BPPG_x_at, WAN01BPPH_at, WAN01BPPM_at, WAN01BPPN_x_at, WAN01BPPQ_at, WAN01BPRV_at, WAN01BPTC_at, WAN01BPTJ_at, WAN01BPTV_at, WAN01BPU8_at, WAN01BPUL_at, WAN01BPXK_at, WAN01BPXQ_x_at, WAN01BPXY_at, WAN01BPY8_at, WAN01BPY9_at, WAN01BPZ6_at, WAN01BQ08_at, WAN01BQ3K_at, WAN01BQ50_at, WAN01BQ7Q_at, WAN01BQ8D x_at, WAN01BQ8G_at, WAN01BQ81_x_at, WAN01BQ80_at, WAN01BQ99_at, WAN01BQ9B_at, WAN01BQ9Z_at, WAN01BQA0_at, WAN01BQBV_at, WAN01BQCP_at, WAN01BQCT_at, WAN01BQD3_at, WAN01BQDB_at, WAN01BQE8_at, WAN01BQGT_at, WAN01BQHM_at, WAN01BQHQ_at, WAN01BQI0_s_at, WAN01BQI1_at, WAN01BQJG_at, WAN01BQJM_x_at, WAN01BQKQ_at, WAN01BQM2_at, WAN01BQM5_x_at, WAN01BQMM_at, WAN01BQMO_at, WAN01BQMY_at, WAN01BQNF_at, WAN01BQNJ_at, WAN01BQNW_at, WAN01BQOB_at, WAN01BQP1_at, WAN01BQP3_at, WAN01BQPE_at, WAN01BQPQ_at, WAN01BQPV_at, WAN01BQPW_at, WAN01BQPX_x_at, WAN01BQQ3_at, WAN01BQQ7_at, WAN01BQQ8_at, WAN01BQQK_at, WAN01BQQN_at, WAN01BQSX_at, WAN01BQT6_at, WAN01BQU6_s_at, WAN01BQUF_at, WAN01BQUP_at, WAN01BQV7x_at, WAN01BQVN_at, WAN01BQWZ_at, WAN01BQX0_at, WAN01BRCD_at, WAN01BSDG_at, WAN01BSSD_at, WAN01BSVG_at, WAN01BSVJ_at, WAN01BSY9_at, WAN01BSYF_at, WAN01BSYQ_at, WAN01BSZN_at, WAN01BT0Y_at, WAN01BT18_at, WAN01BT1B_at, WAN01BT25_x_at, WAN01BT2I_at, WAN01BT2Z_at, WAN01BT3F_at, WAN01BT4Z_at, WAN01BT5N_at, WAN01BT5R_x_at, WAN01BT6H_at, WAN01BT6X_at, WAN01BT6Y_x_at, WAN01BT76_x_at, WAN01BT7O_at, WAN01BT7P_at, WAN01BT7U_at, WAN01BT7Y_at, WAN01BT82_at, WAN01BT83_at, WAN01BT8N_at, WAN01BTAO_at, WAN01BTC0_at, WAN01BTC1_at, WAN01BTCM_at, WAN01BTCY_at, WAN01BTCZ_x_at, WAN01BTD4_at, WAN01BTDR_at, WAN01BTDV_at, WAN01BTE0_at, WAN01BTFI_at, WAN01BTFS_at, WAN01BTG2_at, WAN01BTG6_at, WAN01BTHU_at, WAN01BTHY_at, WAN01BTIA_at, WAN01BTJF_at, WAN01BTJK_at, WAN01BTL8_at, WAN01BTNH_at, WAN01BTO3_at, WAN01BTOS_at, WAN01BTPO_at, WAN01BTQB_at, WAN01BTQS_at, WAN01BTRL_at, WAN01BTRQ_at, WAN01BTRU_x_at, WAN01BTRZ_at, WAN01BTU3_at, WAN01BTUI_at, WAN01BTWH_at, WAN01BTWI_at, WAN01BTWN_at, WAN01BTWO_at, WAN01BTWP_at, WAN01BTWV_at, WANO1BTX7_x_at, WAN01BTZH_at, WAN01BU0Q-seg1_at, WAN01BU0Q-seg2_at, WAN01BU0Q-seg3_at, WAN01BU0Q-seg4_at, WAN01BU0Q-seg6_s_at, WAN01BU2B_s_at, WAN01BU2V_at, WAN01BU2W_x_at, WAN01BU30_at, WAN01BU33_x_at, WAN01BU34_at, WAN01BU35_at, WAN01BU38_x_at, WAN01BU3A_at, WAN01BU3M_at, WAN01BU3T_at, WAN01BU6J_at, WAN01BUAR_at, WAN01BUB0_at, WAN01BUBB_s_at WAN01BUBL_at WAN01BUBX_at, WAN01BUCD_at, WAN01BUCI_at, WAN01BUCJ_at, WAN01BUCK_at, WAN01BUCL_at, WAN01BUD8_at, WAN01BUDD_at, WAN01BUDN_at, WAN01BUDO_at, WAN01BUDP_at, WAN01BUDS_at, WAN01BUDU_at, WAN01BUDW_at, WAN01BUE4_at, WAN01BUE7_at, WAN01BUEG_at, WAN01BUFT_x_at, WAN01BUHJ_at, WAN01BUHO_x_at, WAN01BUID_at, WAN01BUIE_at, WAN01BUIJ_x_at, WAN01BUIN_at, WAN01BUIS_x_at, WAN01BUIU_x_at, WAN01BUIV_x_at, WAN01BUJ8_at, WAN01BUJD_at, WAN01BUJF_at, WAN01BUJG_at, WAN01BUJK_at, WAN01BULB_at, WAN01BULO_x_at, WAN01BUM7_at, WAN01BUMI_at, WAN01BUNG_at, WAN01BUNO_at, WAN01BUNR_at, WAN01BUON_at, WAN01BUOX_at, WAN01BUOY_at, WAN01BUQI_at, WAN01BURL_at, WAN01BUSX_at, WAN01BUT1_s_at, WAN01BUTL_at, WAN01BUUF_at, WAN01BUUJ_at, WAN01BUUK_at, WAN01BUV3_at, WAN01BUVL_at, WAN01BUVW_at, WAN01BUVX_at, WAN01BUWT_at, WAN01BUWU_at, WAN01BUWX_x_at, WAN01BUX0_at, WAN01BUX3_at, WAN01BUX4_at, WAN01BUXC_at, WAN01BUYQ_at, WAN01BUYZ_at, WAN01BUZL_at, WAN01BV0F_at, WAN01BV10_x_at, WAN01BV1J_at, WAN01BV1L_at, WAN01BV1S_at, WAN01BV21_at, WAN01BV3G_at, WAN01BVDE_at, WAN01BVEQ_at, WAN01BW3M_x_at, WAN01BWRZ_at, WAN01BWZ7_at, WAN01BX0L_at, WAN01BX0Q_at, WAN01BX0R_at, WAN01BX0W_at, WAN01BX10_at, WAN01BX13_at, WAN01BX1B_x_at, WAN01BX1F_x_at, WAN01BX2C_at, WAN01BX4S_at, WAN01BX6J_x_at, WAN01BX7O_at, WAN01BX7T_at, WAN01BX9C_x_at, WAN01BXA6_at, WAN01BXAD_at, WAN01BXAO_at, WAN01BXAQ_at, WAN01BXAS_at, WAN01BXAT_at, WAN01BXAU_at, WAN01BXBA_x_at, WAN01BXBJ_at, WAN01BXC6_x_at, WAN01BXDL_at, WAN01BXE3_x_at, WAN01BXFG_at, WAN01BXFK_at, WAN01BXGF_x_at, WAN01BXL3_at, WAN01BXQ2_at, WAN01BXQC_x_at, WAN01BXQZ_at, WAN01BXSQ_x_at, WAN01BXTO_at, WAN01BXVP_at, WAN01BXY7_at, WAN01BY06_at, WAN01BY0E_at, WAN01BY0M_x_at, WAN01BY26_at, WAN01BY31_at, WAN01BY3W_at, WAN01BY5D_at, WAN01BY5G_at, WAN01BY84_x_at, WAN01BY8K_at, WAN01BYE7_x_at, WAN01BYEP_at, WAN01BYF1_at, WAN01BYHK_at, WAN01BYK5_at, WAN01BYLK_at, WAN01BYLU_at, WAN01BYLV_x_at, WAN01BYNC_x_at, WAN01BYP5_at, WAN01BYTK_at, WAN01BYTU_x_at, WAN01BYU4_at, WAN01BYV4_at, WAN01BYWV_x_at, WAN01BYWW_at, WAN01BYWY_x_at, WAN01BYX5_at, WAN01BYXJ_at, WAN01BYXK_at, WAN01BYZP_x_at, WAN01BZ3A_at, WAN01BZ3H_at, WAN01BZ41_at, WAN01BZ42_at, WANO1BZ43_at, WAN01BZ44_at, WAN01BZ45_at, WAN01BZ47_at, WAN01BZ48_at, WAN01BZ49_at, WAN01BZ4A_at, WAN01BZ4R_at, WAN01BZ50_at, WAN01BZ51_at, WAN01BZ52_at, WAN01BZ54_at, WAN01BZ55_at, WAN01BZVA_at, WAN01BZZL_at, WAN01COR1_x_at, WAN01C0U3_at, WAN01C0YK_at, WAN01C1E4_at, WAN01C1EJ_at, WAN01C1PZ_at, WAN01C1RL_x_at, WAN01C1RM_at, WAN01C1SB_x_at, WAN01C1ST_s_at, WAN01C26O_at, WAN01C28I_at, WAN01C299_at, WAN01C2H9_at, WAN01C2HO_at, WAN01C2TP_at, WAN01C2V3_at, WAN01C2V7_at, WAN01C3B5_at, WAN01C3MI_at, WAN01C3NL_at, WAN01C3XV_at, WAN01C3ZF_at, WAN01C3ZO_at, WAN01C401_x_at, WAN01C45G_at, WAN01C4TN_at, WAN01C4UE_at, WAN01C4UG_at, WAN01C4US_at, WAN01C4UT_at, WAN01C4VF_at, WAN01C4VG_at, WAN01C52T_at, WAN01C5GK_at, WAN01C5GL_s_at, WAN01C617_at, WAN01C7GQ_at, WAN01C7NC_at, WAN01C7X8_x_at, WAN01C8DX_x_at, WAN01C8MO_at, WAN01C8OH_x_at, WAN01C8OY_at, WAN01C8P0_at, WAN01C8P5_at, WAN01C8TY_at, WAN01C903_at, WAN01C₉₀H_x_at, WAN01C9HD_x_at, WAN01C9JL_at, WAN01C9JM_at, WAN01C9JR_at, WAN01C9 KB_at, WAN01C9S6_x_at, WAN01C9TR_at, WAN01CA3W_s_at, WAN01CA8O_at, WAN01CAIK_at, WAN01CASJ_s_at, WAN01CASK_x_at, WAN01CAT8_at, WAN01CAWM_at, WAN01CAX8_x_at, WAN01CAX9_x_at, WAN01CAXC_x_at, WAN01CAXD_x_at, WAN01CAXO_at, WAN01CAXQ_x_at, WAN01CAXR_at, WAN01CAYD_at, WAN01CAYE_at, WAN01CAYF_x_at, WAN01CAYG_x_at, WAN01CAYH_s_at, WAN01CAYJ_at, WAN01CAYO_x_at, WAN01CAZ2_at, WAN01CB8G_at, WAN01CB96_x_at, WAN01CBBB_x_at, WAN01CBBC_x_at, WAN01CBBM_at, WAN01CBE2_x_at, WAN01CBER_s_at, WAN01CBET_s_at, WANO1CBEU_s_at, X03216-cds7_at, X06627-cds4_at, X16298-cds2_at, X53096-cds1_at, X53096-cds2_at, X55185-cds1_x_at, X58434-cds1_at, X75439-cds1_at, X75439-cds3_at, Y07536-cds4_x_at, Y07739-cds1_at, Y07739-cds2_at, Y07740-cds1_at, Y09594-cds1_at, Y13600-cds4_at, Y13766-cds1_at, Y18637-cds2_at, Y18641-cds1_at, Y18653-cds1_x_at, WAN01417K-seg6_x_at, AP001553-cds19_x_at, AB009866-cds37_x_at, AF327733-cds5_at, and Z48003-cds1_at.

The tiling or parent sequences for virulence genes include, but are not limited to, AB037671-cds10_at, AB047089-cds4_at, AF053140-cds2_at, AF210055-cds1_at, AF282215-cds2_at, AF282215-cds4_at, AF288402-cds1-seg1_at, AF288402-cds1-seg2_at, AJ277173-cds1_at, M 7348-cds1_at, AJ309178-cds1_at, AJ309180-cds1_at, AJ309181-cds1_at, AJ309182-cds1_at, AJ309184-cds1_at, AJ309185-cds1_at, AJ309190-cds1_at, AJ311975-cds1_at, AJ311976-cds1_at, AJ311977-cds1_at, AY029184-cds1_at, U10927-cds10_at, M63917-cds1_at, U10927-cds1_at, WAN014A7P-seg1_at, U10927-cds11_at, U10927-cds12_at, U10927-cds13_at, U10927-cds2_at, U10927-cds3_at, U10927-cds4_at, U10927-cds5_at, U10927-cds6_at, U10927-cds7_at, U10927-cds8_at, U10927-cds9_at, M21319-cds1_at, WAN014A7P-seg2_at, WAN014A7Q-seg1_at, WAN014A7Q-seg2_at, WAN014A7R-seg1_at, WAN014A7Y-seg1_at, WAN014A7Y-seg2_at, WAN014FR8_at, WAN014FRP_at, WAN014FRU_at, WAN014FSL_at, WAN014FTD_at, WAN014FT0_at, WAN014FU6_at, WAN014FUA_at, WAN014FUF_at, WAN014FV5_at, WAN014FVP_at, WAN014FW9_at, WAN014FWE_at, WAN014FX0_at, WAN014FZ0_at, WAN014G2B_at, WAN014G2E_at, WAN014G2F_at, WAN014G32_at, WAN014G34_at, WAN014G3L_at, WAN014G3M_at, WAN014G3N_at, WAN014G3O_at, WAN014G5F_at, WAN014G7H_at, WAN014G7Q_at, WAN014G7Z_at, WAN014GAU_at, WAN014GAY_at, WAN014 GB1_at, WAN014 GB2_at, WAN014 GB3_at, WAN014GC9_at, WAN014GCB_at, WAN014GCM_at, WAN014GCN_at, WAN014GCP_at, WAN014GCR_at, WAN014GCT_at, WAN014GCV_at, WAN014GD6_at, WAN014GF4_at, WAN014GF6_at, WAN014GF9_at, WAN014GFA_at, WAN014GFB_at, WAN014GK5_at, WAN014GKK_at, WAN014GKN_at, WAN014GKO_at, WAN014GKP_at, WAN014GKQ_at, WAN014GL0_at, WAN014GMS_at, WAN014GQ9_at, WAN014GQG_at, WAN014GQJ_at, WAN014GSO_at, WAN014GSP_at, WAN014GST_at, WAN014GSW_at, WAN014GT1_at, WAN014GUS_at, WAN014GVE_at, WAN014GVO_at, WAN014GW1_at, WAN014GW6_at, WAN014GWE_at, WAN014GWN_at, WAN014GY1_at, WAN014GY3_at, WAN014H5U_at, WAN014HD0_at, WAN014HFQ_at, WAN014HGT_at, WAN014HGV_at, WAN014HGZ_at, WAN014HH1_at, WAN014HH2_at, WAN014HH7_at, WAN014HHS_at, WAN014HHY_at, WAN014HIS_at, WAN014HIT_at, WAN014HJ1_at, WAN014HJJ_at, WAN014HJU_at, WAN014HK2_at, WAN014HK3_at, WAN014HK4_at, WAN014HK5_at, WAN014HKA_at, WAN014HKY_at, WAN014HL5_at, WAN014HLM_at, WAN014HLS_at, WAN014HLW_at, WAN014HM2_at, WAN014HMA_at, WAN014HMJ_at, WAN014HML_at, WAN014HMQ_at, WAN014HMR_at, WAN014HMS_at, WAN014HMT_at, WAN014HQV_at, WAN014HQY_at, WAN014HQZ_at, WAN014HUM_at, WAN014HUN_at, WAN014HVC_at, WAN014HVM_at, WAN014HVN_at, WAN014HVW_at, WAN014HXE_at, WAN014HYX_at, WAN014I06_at, WAN014I2M_at, WAN014I2T_at, WAN014I3E_at, WAN014I40_at, WAN014I4K_at, WAN014I59_at, WAN014I5T_at, WAN014I6E_at, WAN014I7K-seg1_at, WAN014I7K-seg2_at, WAN014I7K-seg3_at, WAN014I7K-seg4_at, WAN014IMJ_at, WAN014IMK_at, WAN014INH_at, WAN014INI_at, WAN014IOV-seg1_at, WAN014IOW-seg2_at, WAN014IOX-seg3_at, WAN014IP2_at, WAN014IP3_at, WAN014IP5_at, WAN014IP6_at, WAN014IP7_at, WAN014IPC_at, WAN014IPD_at, WAN014IPE_at, WAN014IPF_at, WAN014IPG_at, WAN014IPH_at, WAN014IPI_at, WAN014IPJ_at, WAN014IPR_at, WAN014IPZ_at, WAN014IQ0_at, WAN014IQ1_at, WAN014IQ2_at, WAN014IQZ_at, WAN014IR0_at, WAN014IRW_at, WAN014ITM_at, WAN014ITN_at, WAN014ITV_at, WAN014ITW_at, WAN014IU3_at, WAN014IUC_at, WAN014IUU_at, WAN014IUV_at, WAN014IUW_at, WAN014IV4_at, WAN014IVU_at, WAN014IW4_at, WAN014IWK_at, WAN014IWL_at, WAN014IWM_at, WAN014IWN_at, WAN014IWO_at, WAN014IWP_at, WAN014IWQ_at, WAN01BQD3_at, WAN01BQGT_at, WAN01BQUP_at, WAN01BTJK_at, WAN01BUDN_at, WAN01BUDO_at, WAN01BUDP_at, WAN01BUE4_at, WAN01BUNR_at, WAN01BUXC_at, WAN01BV1J_at, WAN01BX2C_at, WAN01BYXJ_at, WAN01BYXK_at, WAN01CAT8_at, D83951-cds2_at, and WANO1CAZ2_at.

The tiling or parent sequences for antimicrobial resistance genes include, but are not limited to, AB037671-cds52_at, J03947-cds1_at, J04551-cds1_at, U19459-cds1_at, WAN014FWE_at, WAN014FZ0_at, WAN014FZG_at, WAN014FZI_at, WAN014G3R_at, WAN014G8O_at, WAN014 GBD_at, WAN014GCI_at, WAN014GCU_at, WAN014GNE_at, WAN014GOC_at, WAN014GUL_at, WAN014GWR_at, WAN014GYZ_at, WAN014HA5_at, WAN014HG1_at, WAN014HGN_at, WAN014HIL_at, WAN014HIQ_at, WAN014HIR_at, WAN014HJ1_at, WAN014HJ2_at, WAN014HJ3_at, WAN014HJ6_at, WAN014HJC_at, WAN014HLT_at, WAN014HMW_at, WAN014HNL_at, WAN014HSN_at, WAN014HSO_at, WAN01416F_at, WAN014IRB_at, WAN014ISL_at, WAN014ITG_at, WAN01BQM2_at, WAN01BQX0_at, WAN01BTG6_at, WAN01C5GK_at, and U82085-cds1_at.

The tiling or parent sequences for genes encoding ribosomal proteins include, but are not limited to, AF327733-cds5_at, WAN014A7W-3_at, WAN014A7W-5_at, WAN014A7W-M_at, WAN014A7X-3_at, WAN014A7X-5_at, WAN014A7X-M_at, WAN014A81-3_at, WAN014A81-5_at, WAN014A81-M_at, WAN014FRA_at, WAN014FRC_at, WAN014FRD_at, WAN014FRF_at, WAN014FT7_at, WAN014FT9_at, WAN014FXU_at, WAN014FYL_at, WAN014G6L_at, WAN014GES_at, WAN014GUP_at, WAN014GVF_at, WAN014GVM_at, WAN014H0O_at, WAN014H1V_at, WAN014H29_at, WAN014H₂C_at, WAN014H2D_at, WAN014H₂F_at, WAN014H2O_at, WAN014H2Q_at, WAN014H2S_at, WAN014H6M_at, WAN014H7Z_at, WAN014H85_at, WAN014H8Z_at, WAN014H9O_at, WAN014HBQ_at, WAN014HBR_at, WAN014HBV_at, WAN014HDA_at, WAN014HDC_at, WAN014HKO_at, WAN014HVK_at, WAN014I0S_at, WAN014I2E_at, WAN014I2L_at, WAN014I3I_at, WAN014I4A_at, WAN014I4I_at, WAN014I58_at, WAN014I5B_at, WAN014I5K_at, WAN014I5O_at, WAN014I5Q_at, WAN014I61_at, WAN014I63_at, WAN014I65_at, WAN014I67_at, WAN014I69_at, WAN014I6B_at, WAN014I6D_at, WAN014I6G_at, WAN014I6I_at, WAN014I6K_at, WAN014I6L_at, WAN014I6O_at, WAN014I6S_at, WAN014I6T_at, WAN014I6W_at, WAN014I6Y_at, and WAN014I70_at.

Table 4 lists exemplary tiling or parent sequences for multilocus sequence typing (MLST) genes, leukotoxin genes, and agrB genes.

TABLE 4
Tiling Sequences for MLST, Leukotoxin, and AgrB Genes

| MLST Gene | Leukotoxin | AgrB |
|---|---|---|
| WAN014GB6_at | WAN014GAU_at | AF210055-cds1_at |
| WAN014GV5_at | WAN014GAY_at | AF282215-cds2_at |
| WAN014H4H_at | WAN014GB3_at | WAN014IPZ_at |
| WAN014H91_at | WAN014HH1_at | WAN014IQ0_at |
| WAN014HDV_at | WAN014HH2_at | WAN014IQ1_at |
| WAN014I0O_at | WAN014HL5_at | WAN014IQ2_at |
| WAN014I60_at | WAN014HMJ_at | |
| | WAN014HML_at | |
| | WAN014HUM_at | |
| | WAN014IUC_at | |

### Example 2

### Analysis of the Accuracy of the Nucleic Acid Array of Example 1

An analysis was conducted to confirm the performance of the nucleic acid array of Example 1 with respect to seven sequenced Staphylococcus aureus genomes, i.e., COL, N315, Mu50, EMRSA-16, MSSA-476, 8325, and MW2. Each tiling sequence in Table C was derived from the transcript(s) or intergenic sequence(s) of one or more Staphylococcus aureus strains. As used herein, if all of the oligonucleotide probes for a tiling sequence are present in the genome of a Staphylococcus aureus strain, then the tiling sequence is theoretically predicted to be “present” in the genome. The theoretical predictions were compared to the actual results of DNA hybridization experiments. Table 5 compares the results of the theoretical predictions for the seven sequenced Staphylococcus aureus strains to the results of actual hybridization experiments using the nucleic acid array of Example 1.

TABLE 5
Comparison of Theoretical and Actual Calls

| | Number of Theoretical | Number of Theoretical Presents |
|---|---|---|
| Strain | Present Calls | Called Absent or Marginal |
| EMRSA-16 | 3,570 | 9 |
| MSSA-476 | 4,275 | 6 |
| 8325 | 4,394 | 7 |
| Mu50 | 6,214 | 6-7 |
| N315 | 6,218 | 8 |
| MW2 | 4,140 | 6 |
| COL | 4,380 | 251 |

Among the seven sequenced Staphylococcus aureus strains, six strains (except COL) showed fewer than 0.25% “absent” or “marginal” calls compared to the predictions. Predicted “present” calls were higher for N315 and Mu50 because the intergenic regions on the nucleic acid array were derived from N315 only. The genome of Mu50 is similar to that of N315.

COL (NARSA 0) was found to have 251 tiling sequences called “absent” or “marginal” but theoretically predicted to be “present.” However, when COL was obtained from other sources, it was found to behave as expected. See Table 6. NARSA 0 was the original strain tested. NARSA 1 and NARSA 2 are derived from individual colonies of a second sample of the COL strain from NARSA. The number of “absent” and “marginal” calls for NARSA 1 was similar to that of NARSA 0, while NARSA 2 has only few “absent” or “marginal” calls. Likewise, other COL colonies (Tomasz, Foster, and Novick) have few “absent” or “marginal” calls. This result suggested that the NARSA O and NARSA 1 colonies were contaminated with non-COL strain(s). This was subsequently confirmed by the strain repository. The NARSA 1 strain was the contaminant, and the NARSA 0 strain included a mixture of two strains, COL and NARSA 1. Thus, the nucleic acid array of Example 1 can be used to detect strain contamination.

TABLE 6
Number of Theoretical Presents Called Absent or Marginal
for Different COL Colonies

| | Number of Theoretical Presents |
|---|---|
| Source | Called Absent or Marginal |
| NARSA 0 | 251 |
| NARSA 1 | 230 |
| NARSA 2 | 6 |
| Tomasz | 5 |
| Foster | 5 |
| Novick | 5 |

The nucleic acid array of Example 1 also includes a substantial number of false positive probe sets which produce significant cross-hybridization of alleles. Table 7 shows excess “present” calls for each strain listed in Table 1 as well as strain MW2. Cross hybridization adds considerable utility for strain typing. This is because the signal obtained in a DNA hybridization experiment is expected to be proportional to the degree of sequence similarity to the probe(s). Thus, the nucleic acid array of Example 1 can potentially distinguish strains with perfectly matched sequence from strains containing single or multiple nucleotide substitutions for any particular gene.

TABLE 7
Excess “Present” Calls

| Strain | Excess Present Calls |
|---|---|
| COL | 2,301 |
| MRSA | 2,664 |
| MSSA | 2,244 |
| 8325 | 2,075 |
| MW2 | 2,336 |
| Mu50 | 675 |
| N315 | 545 |

### Example 3

### Sample Preparation for Monitoring Gene Expression

Total Staphylococcus aureus RNA is isolated from a control condition or a test condition. Under the test condition, bacterial cells have been either differentially treated or have a divergent genotype. cDNA is synthesized from total RNA of the control or test sample as follows. 10 μg total RNA is incubated at 70° C. with 25 ng/μl random hexamer primers for 10 min followed by 25° C. for 10 min. Mixtures are then chilled on ice. Next, 1×cDNA buffer (Invitrogen), 10 mM DTT, 0.5 mM dNTP, 0.5 U/μl SUPERase-In (Ambion), and 25U/μl SuperScript II (Invitrogen) are added. For cDNA synthesis, mixtures are incubated at 25° C. for 10 min, then 370C for 60 min, and finally 42° C. for 60 min. Reactions are terminated by incubating at 70° C. for 10 min and are chilled on ice. RNA is then chemically digested by adding 1N NaOH and incubation at 65° C. for 30 min. Digestion is terminated by the addition of 1N HCl. cDNA products are purified using the QIAquick PCR Purification Kit in accordance with the manufacturer's instructions. Next, 5 μg of cDNA product is fragmented by first adding 1× One-Phor-All buffer (Amersham Pharmacia Biotech) and 3U DNase I (Amersham Pharmacia Biotech) and then incubating at 37° C. for 10 min. DNase I is then inactivated by incubation at 98° C. for 10 min. Fragmented cDNA is then added to 1× Enzo reaction buffer (Affymetrix), 1×CoCl₂, Biotin-ddUTP and 1× Terminal Transferase (Affymetrix). The final concentration of each component is selected according to the manufacturer's recommendations. Mixtures are incubated at 37° C. for 60 min and then stopped by adding 2 μl of 0.5 M EDTA. Labeled fragmented cDNA is then quantitated spectrophotometrically and 1.5 μg labeled material is hybridized to the nucleic acid array at 45° C. for 15 hr.

Staphylococcus aureus mRNA or cRNA can also be used for nucleic acid hybridization. Staphylococcus aureus mRNA or cRNA can be enriched, fragmented, and labeled according to the prokaryotic sample and array processing procedure described in Genechip® Expression Analysis Technical Manual (Affymetrix, Inc. 2002).

### Example 4

### Sample Preparation for Genotyping Staphylococcus aureus

Staphylococcus aureus strains are grown overnight in a 2-ml trypticase soy broth culture. Cells are harvested and lysed in a Bio101 FastPrep bead-beater (2×20 s cycles). Chromosomal DNA is prepared using the Qiagen DNeasy Tissue kit following the manufacturer's instructions. Approximately 10 μg of DNA is made up to a 60 μl volume in nuclease free water. 20 μl 1N NaOH is added to remove residual RNA and the mixture is incubated at 65° C. for 30 min. 20 μl of 1N HCl is added to neutralize the reaction. The DNA is concentrated by ethanol precipitation using ammonium acetate and resuspended in a 47 μl volume followed by a 5 min boiling step to denature the double-stranded DNA. The DNA is quantified by reading the absorbance at 260 nm. 40 μl of DNA is fragmented by treatment with DNase (0.6 U/μg DNA) in the presence of 1× One-Phor-All buffer (Amersham Pharmacia) in a total volume of 50 μl for 10 min at 37C followed by a 10 min incubation at 98° C. to inactivate the enzyme. 39 μl of fragmented DNA is end-labeled with biotin using the Enzo Bioarray Terminal Labeling kit (Affymetrix). 1.5 μg of labeled DNA is hybridized overnight to the custom nucleic acid array of Example 1 in a mixture containing Oligo B2 (Affymetrix), herring sperm DNA, BSA and a standard curve reagent.

### Example 5

### Hierarchical Clustering of Imperfect ORFS

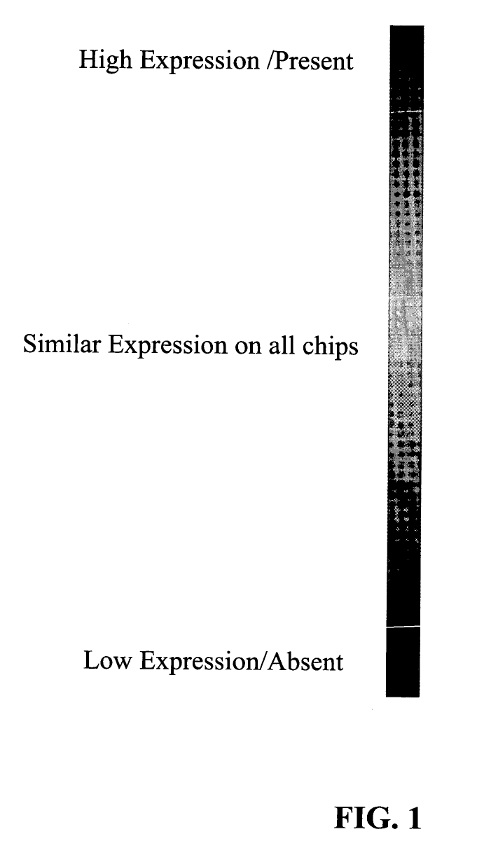

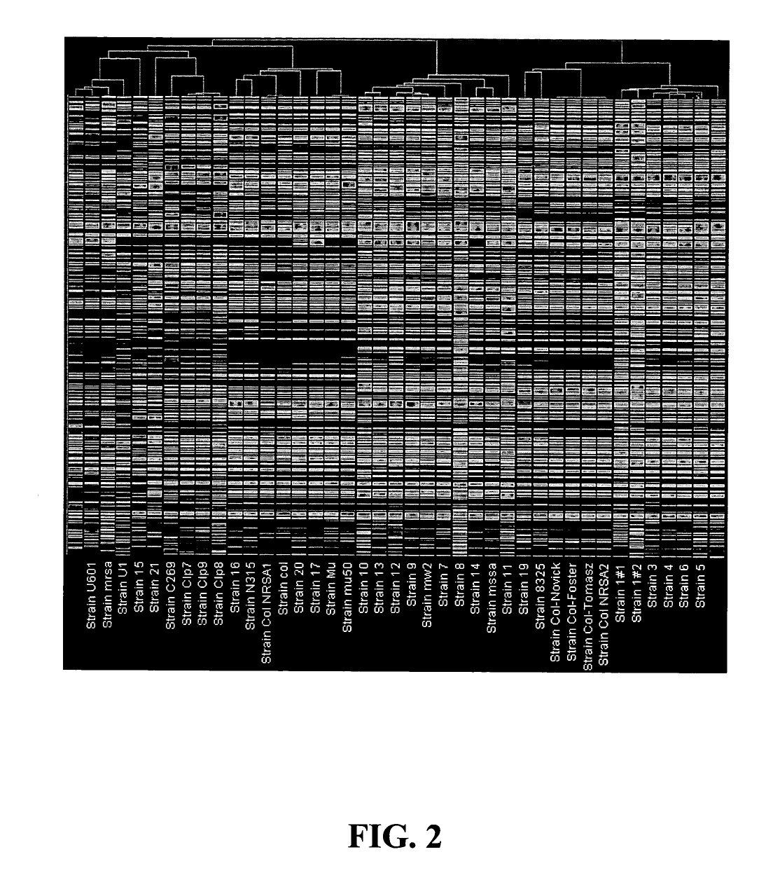

DNA samples were prepared from different Staphylococcus aureus strains or isolates according to the method described in Example 4. The samples were then individually hybridized to the custom nucleic acid array of Example 1. The hybridization signals were normalized by dividing each gene's signal by the median of array intensity and the median of gene intensity across all arrays. FIG. 1 shows the color scale of each gene's distance from the mean value for that gene over all arrays. “Present” is denoted in red and “absent” in blue. Yellow indicates similar signals from all strains for a particular gene. FIG. 2 illustrates an unsupervised hierarchical clustering using normalized signals of 2,059 “imperfect ORFs.” “Imperfect ORFs” were selected for the basis of the clustering because they provide more variation than the “perfect” ORFs which have high sequence identities among all genomes in Table 1. The intergenic sequences were omitted because they were derived from a single strain, and might have biased the clustering algorithm.

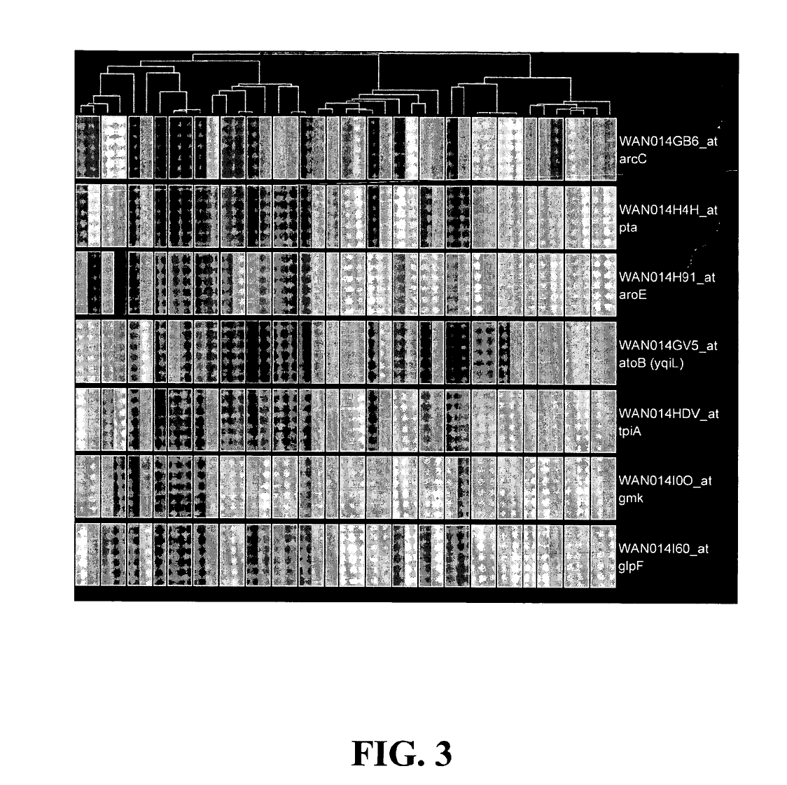

Clustering was performed on 41 Staphylococcus aureus strains/clones, including the seven sequenced genomes, the variant COL strains, 21 strains from the Centers for Disease Control and Prevention, and 6 additional strains from Wyeth's collection. Some were done in duplicate. These strains/clones are listed consecutively along the horizontal axis of FIG. 2. The same set of strains/clones in the same order is used for the horizontal axis of FIGS. 3-7.

FIG. 2 shows that different strains exhibit distinguishable hybridization patterns. Isolates from the same strain, such as Col-Novick, Col-Foster, Col-Tomasz, and Col NRSA2 (i.e., NARSA 2), show similar hybridization patterns. Thus, the nucleic acid arrays of the present invention can be used for typing or identifying different Staphylococcus aureus strains. As appreciated by those skilled in the art, the 2,059 “imperfect ORFs” can be replaced by other genes to generate similar strain-specific hybridization patterns. The nucleic acid arrays of the present invention can be used to generate the complete genotype of a bacterial strain in one step.

### Example 6

### MLST and Virulence Gene Profiles

Multilocus sequence typing (MLST) is a method of characterizing bacterial isolates on the basis of the sequence fragments of seven housekeeping genes. See M. C. Enright et al., JOURNAL OF CLINICAL MICROBIOLOGY, 38: 1008-1015 (2000). These seven genes are acetyl-CoA acetyltransferase, carbamate kinase, phosphotransacetylase, shikimate 5-dehydrogenase, triosephosphate isomerase, guanylate kinase, and glycerol kinase. The tiling sequences for these seven genes are listed in Table 4. Each of these seven genes has many alleles, and different isolates are highly unlikely to have the same allelic profile by chance. FIG. 3 shows the normalized hybridization signals of the seven MLST genes. The samples were prepared using the method described in Example 4. The dendrogram tree and the horizontal axis in FIG. 3 are identical to those in FIG. 2. The yellow color indicates that a gene is present in all strains. FIG. 3 captured the conserved regions of the MLST genes. Probe sets can also be designed to capture the more variable regions in the MLST genes.

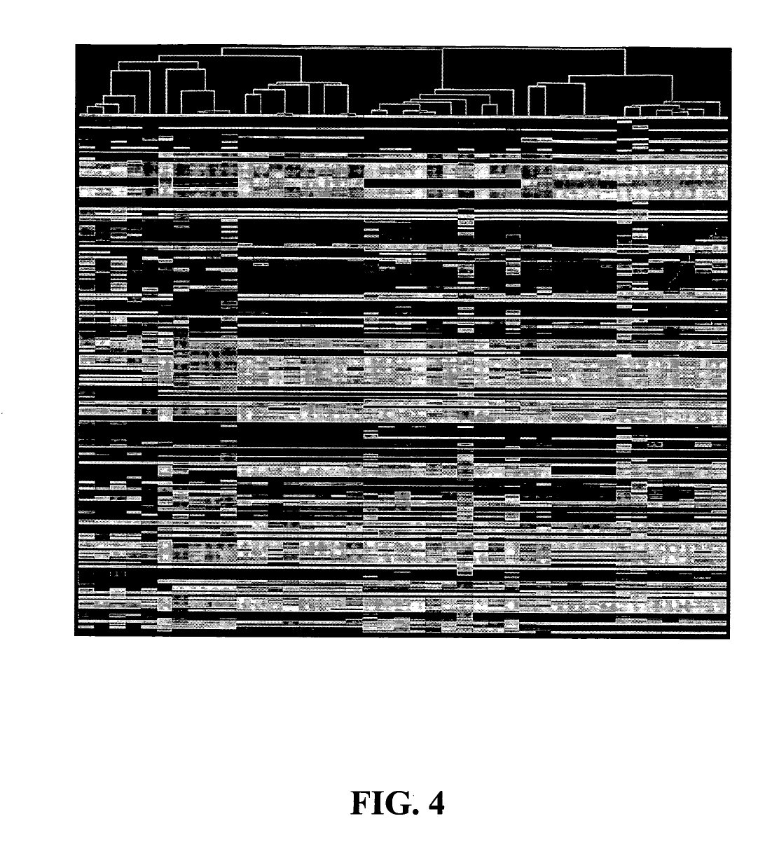

FIG. 4 illustrates the profiles of 259 virulence genes. The virulence genes in FIG. 4 include those that are present in all Staphylococcus aureus strains (yellow), and those that are present in some strains (red) but absent in others (blue). Virulence gene profiles can be used to associate particular strains with particular Staphylococcus aureus symptoms, as specific virulence genes are known to be associated with particular manifestations of disease.

### Example 7

### Panton-Valentine Leukocidin and AgrB Gene Profiles

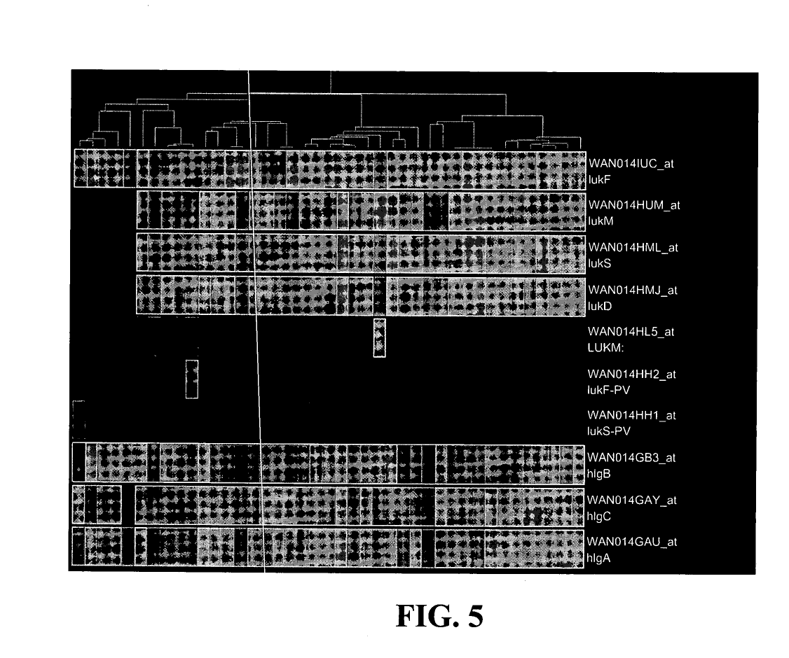

Studies have shown that certain community-acquired methicillin-resistant Staphylococcus aureus (CA-MRSA) strains contain the Panton-Valentine leukocidin (PVL) genes. See P. Dufour et al., CID 35: 819-824 (2002). The PVL genes encode virulence factors associated with primary skin infections (e.g., furunculosis) and severe necrotizing pneumonia. The combination of methicillin-resistance and the PVL determinant creates superadapted Staphylococcus aureus strains. FIG. 5 shows the profiles of PVL genes and other leukotoxin genes. The samples were prepared using the method described in Example 4. The horizontal axis in FIG. 5 is identical to that in FIG. 2, and represents a variety of Staphylococcus aureus strains/clones. PVL genes (lukF-PV and lukS-PV) were present in only a small subset of strains (red). Other leukotoxins (such as lukF, lukM, lukS, lukD, hlgB, hlgC, and hlgA) were present in most or all strains that were being tested. It has been reported that lukE-lukD genes do not appear to be associated with any specific type of infection. See P. Dufour et al., supra.

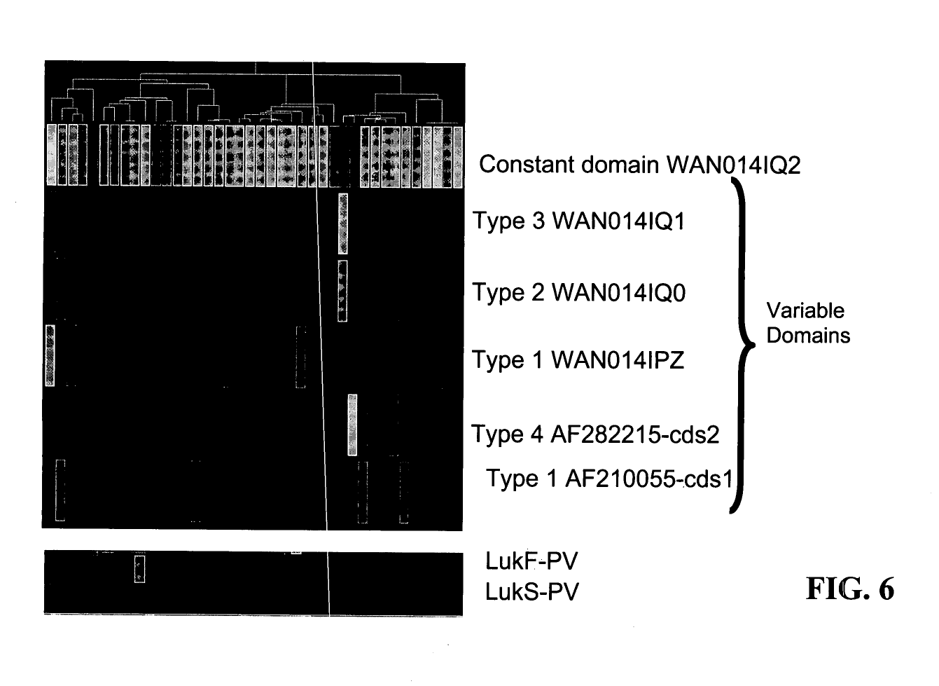

FIG. 6 depicts the association of PVL with two types of agrB. The top row in FIG. 6 shows the profile of the constant N-terminal domain of agrB, which is present in all strains. The next five rows are qualifiers interrogating four agrB types. Type 1 is itself variable and separated into two clusters. PVL genes (lukF-PV and lukS-PV) are associated with agrB types 1 and 3. AgrB encodes a transmembrane protein which has proteolytic activity and can act on a precursor quorum sensing autoinducing peptide.

### Example 8

### Exfoliative Toxin Gene Profiles

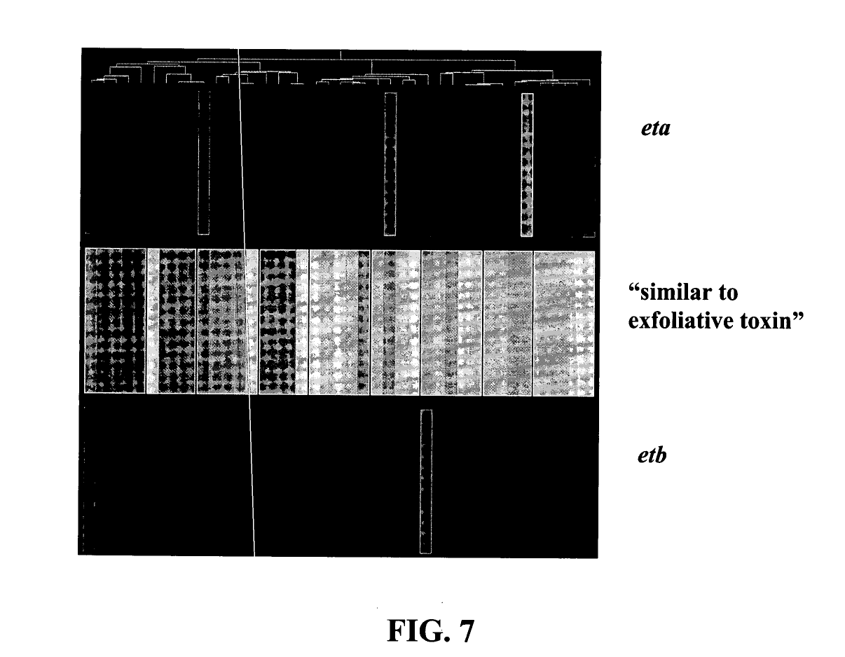

Staphylococcal Scalded Skin Syndrome (SSSS) is a syndrome of acute exfoliation of the skin. SSSS is also known as Ritter von Ritterschein disease in newborns, staphylococcal epidermal necrolysisis, Ritter disease, or Lyell disease. It is caused by an exfoliative toxin. At least two types of exfoliative toxin are known—namely, type A (“eta”) and type B (“etb”). Type A is more prevalent in the United States. FIG. 7 illustrates the profiles of eta and etb in various Staphylococcus aureus strains/clones. The horizontal axis in FIG. 7 is identical to that in FIG. 2, and represents the same set of Staphylococcus aureus strains/clones in the same order. The “eta,” “similar to exfoliative toxin,” and “etb” genes correspond to qualifiers WAN014HKY, WAN014GVE, and M17348-cds, respectively.

As shown by the bottom row in FIG. 7, strains Clp7, Clp8, and Clp9 contain the etb gene (red). Etb gene is absent from other strains. Strains Clp7, Clp8, and Clp9 were isolated from a single patient over the course of one week. These strains cluster closely together. See FIG. 2 and the dendrogram tree.

As shown by the top row in FIG. 7, strain C269 contains the eta gene (red). The dendrogram tree shows that strains Clp7, Clp8, and Clp9 are closely related to strain C269.

The middle row in FIG. 7 illustrates the profile of a gene annotated as “similar to exfoliative toxin” in the TIGR annotation of the COL genome. This gene is present in all strains, suggesting it is not associated with SSSS. FIG. 7 indicates that the exfoliative toxin genes are rare among Staphylococcus aureus strains or isolates.

### Example 9

### Microarray-Based Analysis of the Staphylococcus aureus σ^{B}-Regulon

Microarray-based analysis of the transcriptional profiles of the genetically distinct Staphylococcus aureus strains COL, GP268, and Newman indicate that a total of 251 ORFs are influenced by σ^{B} activity. While σ^{B} was found to positively control 198 genes by a factor of ≧2 in at least two out of the three genetic lineages analyzed, 53 ORFs were repressed in the presence of σ^{B}. Gene products that were found to be influenced by σ^{B} are putatively involved in all manner of cellular processes, including cell envelope biosynthesis and turnover, intermediary metabolism, and signalling pathways. Most of the genes/operons identified as upregulated by σ^{B} were preceded by a nucleotide sequence that resembled the σ^{B} consensus promoter sequence of Bacillus subtilis. A conspicuous number of virulence-associated genes were identified as regulated by σ^{B} activity, with many adhesins upregulated and prominently represented in this group, while transcription of various exoproteins and toxins were repressed. The data presented in this Example suggest that the σ^{B} of S. aureus controls a large regulon, and is an important modulator of virulence gene expression that might act conversely to RNAIII, the effector molecule of the agr locus. This alternative transcription factor may be of importance for the invading pathogen to fine-tune its virulence factor production in response to changing host environments. Therefore, modulation of the expression or protein activity of σ^{B} or the genes downstream thereto may be used to fight or control Staphylococcus aureus infections.

Introduction

Transcription of DNA into RNA is catalyzed by RNA polymerase. In bacteria, one RNA polymerase generates nearly all cellular RNAs, including ribosomal, transfer, and messenger RNA. This enzyme consists of six subunits, α₂ββ′ωσ, with α₂ββ′ω forming the catalytically competent RNA polymerase core enzyme (E). The core is capable of elongation and termination of transcription, but it is unable to initiate transcription at specific promoter sequences. The σ subunit, which when bound to E forms the holoenzyme (E-σ), directs the multi-subunit complex to specific promoter elements and allows efficient initiation of transcription. Therefore, C factors provide an elegant mechanism in eubacteria to allow simultaneous transcription of a variety of genetically unlinked genes, provided all these genes share the same promoter specificities.

In addition to the housekeeping sigma subunit, σ⁷⁰ or σ^{A}, most bacteria produce one or more additional σ subunits, termed “alternative σ factors”, which direct the respective E-σ complex to distinct classes of promoters that contain alternative σ factor-specific sequences. At least six alternative σ factors are produced by the enteric bacterium Escherichia coli. Genomic sequence analysis suggests that many alternative σ factors also exist in a number of other pathogenic species such as Treponema palladium (4 alternative σ factors), Vibro cholerae (7 alternative σ factors), Mycobacterium tuberculosis (12 alternative σ factors), and Pseudomonas aeruginosa (23 alternative σ factors). Two alternative σ factors, σ^{B} and σ^{H}, have been identified in Staphylococcus aureus.

The S. aureus alternative transcription factor σ^{B} has been shown to be involved in the general stress response. σ^{B} also directly or indirectly influences the expression of a variety of genes, including many associated with virulence, such as α-hemolysin, clumping factor, coagulase, fibronectin-binding protein A, lipases, proteases, and thermonuclease. In addition, σ^{B} has been shown to influence the expression of several global virulence factor regulators including, SarA, SarS (syn. SarH1), and RNAIII. However, no effect of σ^{B} on S. aureus pathogenicity has been demonstrated in any in vivo model analyzed to date.

Besides its function in regulating virulence determinants, σ^{B} may play a role in mediating antibiotic resistance. Inactivation of the gene encoding for σ^{B}, sigB, in the homogeneously methicillin-resistant strain COL increases its susceptibility to methicillin, while mutations within the rsbU-defective strain BB255, leading to σ^{B}-hyperproduction, are associated with an increase in glycopeptide resistance. Moreover, σ^{B} was shown to affect pigmentation, to increase resistance to hydrogen peroxide and UV-light, as well as to promote microcolony formation and biofilm production.

The genetic organization of the S. aureus sigB operon closely resembles that of the distal part of the well-characterized homologous operon of the soil-borne gram-positive bacterium Bacillus subtilis. DNA microarray technology-based analysis of the general stress response in B. subtilis identified 127 genes controlled by σ^{B}, and heat shock studies suggest the σ^{B} regulon of this organism to comprise up to 200 genes. Because S. aureus σ^{B} seems to be a pleotrophic regulator that plays a role in a number of clinically relevant processes, a number of investigators have begun characterizing the σ^{B} regulon. Proteomic approaches have identified 27 S. aureus cytoplasmic proteins and one extracellular protein to be under the positive control of σ^{B}, while 11 proteins were found to be repressed by the factor, indicating that the σ^{B} regulon of this pathogen may comprise a much higher number of genes than known to date.

In this Example, DNA microarray-based data from three distinct genetic backgrounds were obtained. These data suggests that the S. aureus σ^{B} influences the expression of at least 251 genes. 198 of these genes are positively controlled by σ^{B}, while 53 genes are repressed in presence of the alternative a factor.

Material and Methods

Bacterial strains, media, and growth conditions: Strains and plasmids used in this Example are listed in Table 8. S. aureus was routinely cultured on sheep blood agar (SBA) or Luria-Bertani (LB) medium with rotary agitation at 200 rpm, at 35° C. Exogenous glucose was not added to the growth medium. When included, antibiotics were used at the following concentrations: ampicillin, 50 mg liter⁻¹; chloramphenicol, 40 mg liter⁻¹.

TABLE 8
Strains and Plasmids

| Strain or plasmid: | Relevant Genotype and Phenotype:^{a} | Reference: |
|---|---|---|
| Strains | | |
| E. coli | | |
| XL1Blue | recA1 endA1 gyrA96 thi-1 hsdR17 | Stratagene |
| | supE44 relA1 lac [F′ proAB | |
| | lacl^{Q} ZΔM15 Tn10 (Tc^{r})] | |
| S. aureus | | |
| BB255 | rsbU; low σ^{B}-activity | |
| COL | mec, high-Mc^{r} clinical isolate; Mc^{r} Tc^{r} | |
| Newman | Clinical isolate, high level of clumping | |
| | factor (ATCC 25904) | |
| IK181 | BB255 ΔrsbUVWsigB; Em^{r} | |
| IK183 | COL ΔrsbUVWsigB; Em^{r} Mc^{r} Tc^{r} | |
| IK184 | Newman ΔrsbUVWsigB; Em^{r} | |
| GP268 | BB255 rsbU⁺; Tc^{r} | |
| Plasmids | | |
| pAC7 | Cm^{r}, expression plasmid containing the | |
| | P_{BAD} promoter and the araC gene (68) | |
| pAC7-sigB | Cm^{r}, 767-bp PCR fragment of the sigB | |
| | ORF from strain COL into pAC7 | |
| pSB40N | Ap^{r}, promoter probe plasmid | |
| pSA0455p | Ap^{r}, 360-bp PCR fragment covering the | |
| | promoter region of the COL homologue of | |
| | ORF N315-SA0455 into pSB40N | |

^{a}Abbreviations are as follows: Ap^{r}, ampicillin resistant; Cm^{r}, chloramphenicol resistant; Em^{r}, erythromycin resistant; Mc^{r}, methicillin resistant; Tc^{r}, tetracycline resistant.

Sampling, RNA isolation, and transcriptional profiling Overnight cultures of S. aureus were diluted 1:100 into fresh pre-warmed LB medium and grown as described above. For experiment one, cultures were grown to an optical density at 600 nm (OD₆₀₀) of 2, at which time RNA samples were prepared as described below. For experiment two, cultures were grown for 9 h, and sample volumes corresponding to 10¹⁰ cells were removed after 1, 3, 5, and 8 h of growth. For RNA isolation, samples were centrifuged at 7,000×g at 4° C. for 5 min, the culture supernatants removed, and the cell-sediments snap-frozen in a dry ice-alcohol mixture. Frozen cells were resuspended in 5 ml of ice-cold acetone/alcohol (1:1), and incubated for 5 min on ice. After centrifugation at 7,000×g and 4° C. for 5 min, cells were washed with 5 ml TE buffer (10 mM TRIS, 1 mM EDTA [pH 8]), and resuspended on ice in 900 μl TE. The cell suspensions were transferred to 2-ml Lysing Matrix B tubes (Bio 101, Vista, Calif.), and the tubes were shaken in an FP120 reciprocating shaker (Bio 101) two times at 6,000 rpm for 20 s. After centrifugation at 14,000×g at 4° C. for 5 min, the supernatants were used for RNA isolation using the RNeasy Midi system (Qiagen, Inc., Valencia, Calif.) according to the manufacturer's recommendations. To remove any contaminating genomic DNA, approximately 125 μg of total RNA was treated with 20 U of DNase I (Amersham Biosciences, Piscataway, N.J.) at 37° C. for 30 min. The RNA was then purified with an RNeasy mini column (Qiagen) following the manufacturer's cleanup protocol. Integrity of the RNA preparations was analyzed by electrophoresis in 1.2% agarose-0.66 M formaldehyde gels. Reverse transcription-PCR, cDNA fragmentation, cDNA terminal labeling, and hybridization of approximately 1.5 μg of labeled cDNA to the nucleic acid arrays of Example 1 were carried out in accordance with the manufacturer's (Affymetrix Inc., Santa Clara, Calif.) instructions for antisense prokaryotic arrays. The nucleic acid arrays were scanned using the Agilent GeneArray laser scanner (Agilent Technologies, Palo Alto, Calif.). Data for biological duplicates were normalized and analyzed by using GeneSpring Version 5.1 gene expression software package (Silicon Genetics, Redwood City, Calif.). Genes that were considered to be upregulated in a σ^{B}-dependent manner were found to demonstrate >2 fold increase in RNA titers in σ^{B} producing conditions in comparison to isogenic non-σ^{B} producing cells. In addition these genes were considered to be “present” by Affymetrix algorithums in the σ^{B} producing strains and demonstrated a significant difference in expression (T-test, with a p-cutoff of at least 0.05). Genes considered downregulated in a σ^{B} dependent manner demonstrated at least a 2-fold reduction in RNA transcript titers in the wildtype as opposed to their isogenic σ^{B}-mutant background and were both considered “present” by Affymetrix criteria in mutant cells and where characterized as having significantly differing amounts of transcripts based on T-tests with a p-cutoff of at least 0.05.

Construction of plasmids pAC7-sigB and pSA0455p: A DNA fragment constituting the sigB open reading frame (ORF) of S. aureus COL was amplified by PCR using an upstream primer containing a Nde I site and a downstream primer containing a Hind III site. The resulting PCR product was digested with Nde I and Hind III and cloned into plasmid pAC7 to obtain pAC7-sigB, which was subsequently transformed by electroporation into E. coli XL1Blue (Stratagene, La Jolla, Calif.). Sequence analysis and comparison confirmed the identity of the construct. For pSA0455p, a DNA fragment representing 360-bp of the N315-SA0455 promoter region of COL was generated by PCR using an upstream primer containing a Bam HI site and a downstream primer containing an Xho I site. The PCR product was digested with Bam HI and Xho I and cloned into promoter probe plasmid pSB40N to obtain pSA0455p. Sequence analysis confirmed the identity of the insert. Plasmid pSA0455p was transformed into E. coli XL1Blue containing either compatible plasmids pAC7-sigB or pAC7.

High-resolution S1 nuclease mapping: For RNA isolation from recombinant E. coli cultures, strains were grown at 37° C. in LB supplemented with ampicillin and chloramphenicol to an OD₆₀₀ of 0.3. At this growth stage, expression of S. aureus sigB was induced by adding 0.0002% (w/v) arabinose, and cultivation was continued for additional 3 h. Isolation of total RNA and high-resolution S1 nuclease mapping were performed as described by Kormanec, METHODS MOL. BIOL., 160: 481-494 (2001). A 450-bp DNA fragment covering the SA0455 promoter region was amplified by PCR from pSA0455p, using a universal oligonucleotide primer labeled at the 5′ end with [γ-³²P]ATP, and mut80 primer. 40 μg of RNA were hybridized to 0.02 pmol of the 5′ end-labeled DNA fragment (approx. 3×10⁶ cpm/pmol of probe), and treated with 100 units of S1-nuclease. The protected DNA fragment was analyzed on a DNA sequencing gel together with G+A and T+C sequencing ladder derived from the end-labeled probe.

Results and Discussion

Identification of σ^{B}-regulated genes: Proteomic approaches and computational analyses, based on the method described by Petersohn, et al., J. BACTERIOL. 181: 5718-5724 (1999), indicate that the σ^{B} regulon of S. aureus comprises many more genes than described to date, suggesting that the regulon may be as large as that of the well-characterized homologous regulon of B. subtilis. In an effort to better define the S. aureus CB regulon, DNA microarray studies were preformed in three genetically distinct backgrounds. DNA microarray technology is a powerful tool to analyze the transcription profiles of the whole genome, provided that all genes are represented on the respective microarray. There is increasing evidence that extensive variation in gene content exists among strains of many pathogenic bacterial species. A genomic comparison of 36 S. aureus strains of divergent clonal lineage identified a very large genetic variation to be present in this pathogen, with approximately 22% of the genome being dispensable. The S. aureus nucleic acid array of Example 1 study includes probes that monitor the expression of virtually all ORFs from six S. aureus genomes, making it an ideal tool to identify almost all transcriptional changes that are caused by the alternative transcription factor σ^{B}.

Two different approaches were chosen in order to identify σ^{B}-dependent genes. In experiment one, the transcriptional profiles of three genetically distinct S. aureus strains harboring an intact sigB operon (COL, Newman, and GP268), and their isogenic ΔrsbUVWsigB mutants were analyzed. For this purpose, total bacterial RNA was obtained from cells that were grown to late exponential growth phase (OD₆₀₀=2), a time point at which σ^{B} has been shown to be active. Comparison of the transcriptional profiles of the sigB⁺ strains to their respective isogenic sigB mutants identified 229 ORFs to be influenced by σ^{B} by a factor of more than two-fold in at least two out of the three genetic backgrounds analyzed (Tables 9 and 10). While the majority of ORFs were positively influenced by σ^{B} (Table 9), as expected for a σ factor, a number of ORFs that were repressed in presence of σ^{B} were also identified (Table 10). Thirty-seven of the genes identified were shown to be regulated by σ^{B} in S. aureus. Twenty-three genes were identified to be influenced by σ^{B} in B. subtilis. This high correlation indicates that the microarray methodology used accurately identified the genes belonging to the σ^{B} regulon of the strains analyzed.

TABLE 9
Genes Upregulated by σ^{B}

| N315 | N315 | | Fold change^{b} | | | |
|---|---|---|---|---|---|---|
| ORF No.^{a} | gene^{a} | N315 description^{a} | COL | Newman | GP268 | σ^{B} consensus^{c,d} |
| N315-SA1984 | asp23 | Alkaline shock protein 23 | Up | Up | Up | yes |
| CAB75732.1 | bbp | Bone sialoprotein-binding | 3.2 | 4.5 | 4.8 | |
| | | protein Bbp | | | | |
| N315-SA2008 | budB | α-acetolactate synthase | Up | Up | Up | yes^{d} |
| N315-SA0144 | cap5A | Capsular polysaccharide | Up | Up | 12.8 | |
| | | synthesis enzyme Cap5A | | | | |
| N315-SA0145 | cap5B | Capsular polysaccharide | Up | Up | 10.8 | |
| | | synthesis enzyme Cap5B | | | | |
| N315-SA0146 | cap5C | Capsular polysaccharide | Up | Up | 8.6 | |
| | | synthesis enzyme Cap8C | | | | |
| N315-SA0147 | cap5D | Capsular polysaccharide | Up | Up | 7.3 | |
| | | synthesis enzyme Cap5D | | | | |
| N315-SA0148 | cap5E | Capsular polysaccharide | Up | Up | 7.5 | |
| | | synthesis enzyme Cap8E | | | | |
| N315-SA0149 | cap5F | Capsular polysaccharide | Up | Up | 7.5 | |
| | | synthesis enzyme Cap5F | | | | |
| N315-SA0150 | cap5G | Capsular polysaccharide | Up | Up | 6.8 | |
| | | synthesis enzyme Cap5G | | | | |
| N315-SA0151 | cap5H | Capsular polysaccharide | Up | Up | 5.1 | |
| | | synthesis enzyme Cap5H | | | | |
| N315-SA0152 | cap5I | Capsular polysaccharide | Up | Up | 5.7 | |
| | | synthesis enzyme Cap5I | | | | |
| N315-SA0153 | cap5J | Capsular polysaccharide | Up | Up | 3.5 | |
| | | synthesis enzyme Cap5J | | | | |
| N315-SA0155 | cap5L | Capsular polysaccharide | Up | Up | 5.1 | |
| | | synthesis enzyme Cap5L | | | | |
| N315-SA0156 | cap5M | Capsular polysaccharide | Up | Up | 4.5 | |
| | | synthesis enzyme Cap5M | | | | |
| N315-SA0157 | cap5N | Capsular polysaccharide | 2.7 | Up | 5.2 | |
| | | synthesis enzyme Cap5N | | | | |
| N315-SA0158 | cap5O | Capsular polysaccharide | 2.6 | Up | 4.2 | |
| | | synthesis enzyme Cap8O | | | | |
| CAA79304 | clfA | Clumping factor A | 35.7 | Up | 7.8 | yes |
| N315-SA2336 | clpL | ATP-dependent Clp proteinase | 17.3 | 13.2 | Up | yes |
| | | chain ClpL | | | | |
| N315-SA2349 | crtM | Squalene desaturase | Up | Up | Up | yes^{d} |
| N315-SA2348 | crtN | Squalene synthase | Up | Up | Up | yes^{d} |
| N315-SA1452 | csbD | HP, sigmaB-controlled gene | 37.0 | Up | Up | yes |
| | | product CsbD (Csb8) | | | | |
| COL-SA1872 | epiE | Epidermin immunity protein | Up | Up | Up | yes^{d} |
| | | EpiE | | | | |
| COL-SA1873 | epiF | Epidermin immunity protein | Up | Up | Up | yes |
| | | EpiF | | | | |
| N315-SA1634 | epiG | Epidermin immunity protein | Up | Up | Up | yes^{d} |
| | | EpiG | | | | |
| N315-SA2260 | fabG | HP, similar to glucose 1- | Up | Up | Up | yes |
| | | dehydrogenase | | | | |
| N315-SA1901 | fabZ | (3R)-hydroxymyristoyl-[acyl | 2.2 | 5.1 | 2.0 | yes^{d} |
| | | carrier protein] dehydratase | | | | |
| N315-SA2125 | hutG | HP, similar to | 3.7 | 14.6 | 2.9 | yes |
| | | formiminoglutamase | | | | |
| N315-SA1505 | lysP | Lysine-specific permease | 2.4 | 7.9 | 2.0 | |
| N315-SA1962 | mtlA | PTS system, mannitol specific | 8.5 | 17.2 | Up | yes^{d} |
| | | IIA component | | | | |
| N315-SA1963 | mtlD | Mannitol-1-phosphate 5- | 8.2 | Up | Up | yes^{d} |
| | | dehydrogenase | | | | |
| N315-SA1902 | murA | UDP-N-acetylglucosamine 1- | 2.2 | 5.1 | 2.0 | yes^{d} |
| | | carboxyvinyl transferase 1 | | | | |
| N315-SA0547 | mvaK1 | Mevalonate kinase | 2.4 | 4.5 | 1.3 | yes |
| N315-SA0548 | mvaD | Mevalonate diphosphate | 3.3 | 7.3 | 1.8 | yes^{d} |
| | | decarboxylase | | | | |
| N315-SA0549 | mvaK2 | Phosphomevalonate kinase | 3.7 | 10.6 | 2.2 | yes^{d} |
| N315-SA1987 | opuD | Glycine betaine transporter | Up | Up | Up | yes |
| | | opuD homologue | | | | |
| N315-SA1871 | rsbV | Anti-σ^{B} factor antagonist | Up | Up | Up | yes |
| N315-SA1870 | rsbW | Anti-σ^{B} factor | Up | Up | Up | yes^{d} |
| N315-SA0573 | sarA | Staphylococcal accessory | 2.9 | 3.8 | 2.0 | yes |
| | | regulator A (Csb35) | | | | |
| N315-SA0108 | sarS | Staphylococcal accessory | 2.6 | 1.1 | 2.1 | yes |
| | | regulator A homologue S | | | | |
| N315-SA0099 | sbtA | HP, similar to transmembrane | Up | Up | Up | |
| | | efflux pump protein | | | | |
| N315-SA1869 | sigB | Alternative transcription factor | Up | Up | Up | yes^{d} |
| | | σ^{B} | | | | |
| N315-SA0456 | spoVG | Stage V sporulation protein G | 4.3 | 9.8 | 3.0 | yes^{d} |
| | | homologue | | | | |
| N315-SA1114 | truB | tRNA pseudouridine 5S | 2.1 | Up | 2.3 | yes |
| | | synthase | | | | |
| N315-SA2119 | ydaD | HP, simialr to dehydrogenase | 4.8 | 33.1 | 16.9 | yes |
| | | (Csb28) | | | | |
| N315-SA0084 | | HP, similar to homo sapiens | Up | Up | 3.0 | yes |
| | | CGI-44 protein | | | | |
| N315-SA0098 | | HP, similar to aminoacylase | Up | Up | Up | |
| N315-SA0102 | | 67 kDa Myosin-crossreactive | Up | Up | Up | yes |
| | | streptococcal antigen | | | | |
| | | homologue | | | | |
| N315-SA0105 | | HP | Up | Up | Up | |
| N315-SA0163 | | HP, similar to cation-efflux | Up | Up | Up | |
| | | system membrane protein | | | | |
| | | CzcD | | | | |
| N315-SA0164 | | HP | Up | Up | Up | yes |
| N315-SA0261 | | HP, similar to rbs operon | 2.5 | Up | Up | yes |
| | | repressor RbsR | | | | |
| N315-SA0296 | | Conserved HP | 7.6 | 20.5 | 3.9 | yes |
| N315-SA0297 | | HP, similar to ABC transporter | 6.3 | 13.1 | 2.8 | yes^{d} |
| | | ATP-binding protein | | | | |
| N315-SA0317 | | HP, similar to | 11.6 | 20.7 | 3.9 | yes |
| | | dihydroflavonol-4-reductase | | | | |
| N315-SA0326 | | Conserved HP | 2.5 | 2.1 | 2.0 | yes |
| N315-SA0327 | | Conserved HP | 2.2 | 2.1 | 2.0 | yes^{d} |
| N315-SA0359 | | Conserved HP | Up | Up | Up | yes |
| N315-SA0360 | | Conserved HP | Up | Up | 77.7 | yes |
| N315-SA0372 | | HP (Csb12) | 1.6 | 3.3 | 2.0 | yes |
| N315-SA0455 | | Translation initiation inhibitor | 3.2 | 6.2 | 2.3 | yes |
| | | homologue | | | | |
| N315-SA0509 | | Conserved HP | 2.0 | 12.1 | 2.0 | |
| N315-SA0528 | | HP, similar to hexulose-6- | 1.8 | 6.8 | 2.0 | yes |
| | | phosphate synthase (Csb4) | | | | |
| N315-SA0529 | | Conserved HP (Csb4-1) | 1.9 | 8.7 | 2.0 | yes^{d} |
| N315-SA0541 | | HP, similar to cationic amino | 11.3 | 14.4 | 7.7 | yes |
| | | acid transporter | | | | |
| N315-SA0572 | | HP, similar to esterase/lipase | Up | Up | Up | yes |
| N315-SA0577 | | HP, similar to FimE | Up | Up | Up | |
| | | recombinase | | | | |
| N315-SA0578 | | HP, similar to NADH | Up | Up | Up | yes |
| | | dehydrogenase | | | | |
| N315-SA0579 | | HP, similar to Na+/H+ | Up | Up | 4.0 | yes^{d} |
| | | antiporter | | | | |
| N315-SA0580 | | HP, similar to Na+/H+ | Up | Up | Up | yes^{d} |
| | | antiporter | | | | |
| N315-SA0581 | | MnhD homologue, similar to | Up | Up | 6.0 | yes^{d} |
| | | Na+/H+ antiporter subunit | | | | |
| N315-SA0582 | | HP, similar to Na+/H+ | Up | Up | 4.0 | yes^{d} |
| | | antiporter | | | | |
| N315-SA0583 | | HP, similar to Na+/H+ | Up | Up | 4.7 | yes^{d} |
| | | antiporter | | | | |
| N315-SA0584 | | Conserved HP | Up | Up | 5.3 | yes^{d} |
| N315-SA0633 | | HP | 2.0 | 8.7 | 2.9 | yes^{d} |
| N315-SA0634 | | Conserved HP | 1.9 | 6.6 | 2.3 | yes^{d} |
| N315-SA0635 | | Conserved HP | 5.1 | 14.8 | 2.8 | yes^{d} |
| N315-SA0636 | | Conserved HP | 5.5 | 22.9 | 2.2 | yes^{d} |
| N315-SA0637 | | Conserved HP | 5.3 | 24.3 | 3.5 | yes |
| N315-SA0658 | | HP, similar to plant-metabolite | 3.0 | 10.5 | 2.5 | yes |
| | | dehydrogenases | | | | |
| N315-SA0659 | | HP, similar to CsbB stress | 3.3 | 10.4 | 2.5 | yes^{d} |
| | | response protein | | | | |
| N315-SA0665 | | Coenzyme PQQ synthesis | 2.1 | 4.5 | 1.8 | |
| | | homologue | | | | |
| N315-SA0666 | | 6-pyruvoyl tetrahydrobiopterin | 2.3 | 5.7 | 2.1 | |
| | | synthase homologue | | | | |
| N315-SA0681 | | HP, similar to multidrug | 2.4 | Up | Up | yes |
| | | resistance protein (Csb29) | | | | |
| N315-SA0721 | | Conserved HP | 4.2 | 10.3 | 2.4 | yes |
| N315-SA0722 | | Conserved HP | 3.4 | 9.4 | 1.5 | yes^{d} |
| N315-SA0724 | | HP, similar to cell-division | 2.5 | 3.8 | 2.5 | yes |
| | | inhibitor | | | | |
| N315-SA0725 | | Conserved HP | Up | Up | Up | |
| N315-SA0740 | | HP | Up | Up | Up | yes |
| N315-SA0741 | | Conserved HP | Up | Up | Up | yes^{d} |
| N315-SA0748 | | HP | 3.0 | Up | 4.8 | yes^{d} |
| N315-SA0749 | | HP | 2.5 | Up | 6.6 | yes |
| N315-SA0751 | | HP | 4.3 | 5.7 | 4.1 | |
| N315-SA0752 | | HP | Up | Up | Up | yes |
| N315-SA0755 | | HP, similar to general stress | Up | Up | Up | yes |
| | | protein 170 | | | | |
| N315-SA0768 | | Conserved HP | 2.0 | 5.6 | 4.5 | |
| N315-SA0772 | | Conserved HP | Up | Up | Up | yes |
| N315-SA0774 | | HP, similar to ABC transporter | 2.1 | 2.0 | 1.4 | yes |
| | | ATP-binding protein | | | | |
| | | homologue (Csb10) | | | | |
| N315-SA0780 | | HP, similar to hemolysin | 3.3 | Up | 2.2 | yes |
| N315-SA0781 | | HP, similar to 2-nitropropane | 2.2 | Up | 2.0 | yes^{d} |
| | | dioxygenase | | | | |
| N315-SA0933 | | HP | 13.1 | 26.9 | 7.1 | yes |
| N315-SA1014 | | Conserved HP | Up | Up | Up | yes |
| N315-SA1057 | | Conserved HP | 2.4 | 3.9 | 3.1 | yes |
| N315-SA1559 | | HP, similar to smooth muscle | 3.6 | 12.1 | 2.1 | yes^{d} |
| | | caldesmon | | | | |
| N315-SA1560 | | HP, similar to general stress | 2.8 | 8.2 | 2.2 | yes |
| | | protein homolog | | | | |
| N315-SA1573 | | HP | 5.9 | 21.0 | 3.0 | yes |
| N315-SA1590 | | HP | 2.0 | 4.3 | 2.1 | yes |
| N315-SA1657 | | Conserved HP | 2.0 | 4.5 | 2.4 | yes |
| N315-SA1671 | | HP (Csb33) | 3.0 | 9.4 | 2.1 | yes |
| N315-SA1692 | | Conserved HP (Csb3) | 1.8 | 5.6 | 4.0 | |
| N315-SA1697 | | HP, simialr to protein-tyrosine | 2.3 | 5.0 | 3.7 | yes |
| | | phosphatase | | | | |
| N315-SA1698 | | HP | 1.3 | 2.9 | 2.0 | yes^{d} |
| N315-SA1699 | | HP, simialr to transporter | 5.0 | 23.1 | 6.1 | yes^{d} |
| N315-SA1814 | | HP, similar to succinyl- | Up | Up | Up | |
| | | diaminopimelate desuccinylase | | | | |
| N315-SA1903 | | Conserved HP | 3.7 | 10.9 | 3.7 | yes^{d} |
| N315-SA1924 | | HP, simialr to aldehyde | 3.7 | 26.1 | 3.2 | yes |
| | | dehydrogenase (Csb24) | | | | |
| N315-SA1942 | | Conserved HP | 2.3 | 7.9 | 3.6 | |
| N315-SA1946 | | Conserved HP (Csb9) | Up | Up | Up | yes |
| N315-SA1961 | | HP, similar to transcription | 9.7 | 8.2 | Up | yes^{d} |
| | | antiterminator BglG family | | | | |
| N315-SA1980 | | Conserved HP | 3.4 | 4.7 | 1.1 | yes^{d} |
| N315-SA1981 | | Conserved HP | 5.7 | 7.7 | 1.6 | yes |
| N315-SA1985 | | HP | Up | Up | Up | yes^{d} |
| N315-SA1986 | | HP | Up | Up | Up | yes |
| N315-SA2006 | | HP, similar to MHC class II | Up | Up | Up | |
| | | analog | | | | |
| N315-SA2101 | | Conserved HP | 2.2 | 3.3 | 1.5 | yes^{d} |
| N315-SA2102 | | Conserved HP | 2.2 | 3.3 | 1.7 | yes |
| N315-SA2104 | | HP, similar to suppressor | 2.1 | 2.2 | 1.8 | yes |
| | | protein SuhB | | | | |
| N315-SA2158 | | HP, similar to TpgX protein | 2.2 | 3.5 | 2.5 | yes |
| N315-SA2203 | | HP, similar to multidrug | 2.1 | 3.9 | 2.2 | yes |
| | | resistance protein | | | | |
| N315-SA2219 | | Conserved HP | Up | Up | 3.0 | yes |
| N315-SA2240 | | HP, similar to para-nitrobenzyl | 1.9 | 2.0 | 2.0 | |
| | | esterase chain A | | | | |
| N315-SA2242 | | Conserved HP | Up | Up | Up | |
| N315-SA2243 | | HP, similar to ABC transporter | Up | Up | Up | |
| | | (ATP-binding protein) | | | | |
| N315-SA2262 | | Conserved HP (Csb7) | Up | Up | Up | yes |
| N315-SA2267 | | HP | 3.0 | Up | 3.9 | yes |
| N315-SA2298 | | Conserved HP | 3.4 | 30.9 | 6.1 | |
| N315-SA2309 | | Conserved HP | 2.0 | 2.5 | 2.9 | |
| N315-SA2327 | | HP, similar to pyruvate | 51.1 | Up | 17.9 | |
| | | oxidase | | | | |
| N315-SA2328 | | Conserved HP | Up | Up | Up | |
| N315-SA2350 | | Conserved HP | Up | Up | Up | yes^{d} |
| N315-SA2351 | | HP, similar to phytoene | Up | Up | Up | yes^{d} |
| | | dehydrogenase | | | | |
| N315-SA2352 | | HP | Up | Up | Up | yes |
| N315-SA2366 | | Conserved HP | 7.3 | Up | 4.5 | yes |
| N315-SA2367 | | Conserved HP | 10.4 | Up | 8.9 | yes |
| N315-SA2374 | | Conserved HP | Up | Up | Up | |
| N315-SA2398 | | HP | Up | Up | Up | yes |
| N315-SA2403 | | Conserved HP | 10.3 | Up | 8.7 | yes |
| N315-SA2440 | | HP | 2.3 | 5.9 | 1.7 | |
| N315-SA2441 | | HP, similar to | 2.5 | 6.6 | 2.0 | |
| | | lipopolysaccharide | | | | |
| | | biosynthesis protein | | | | |
| N315-SA2442 | | Preprotein translocase SecA | 3.5 | 8.5 | 2.0 | |
| | | homologue | | | | |
| N315-SA2451 | | HP | Up | Up | Up | yes |
| N315-SA2452 | | Conserved HP | Up | Up | 3.5 | |
| N315-SA2479 | | Conserved HP | Up | 4.3 | 4.6 | yes |
| N315-SA2485 | | HP | Up | Up | Up | yes |
| N315-SA2488 | | HP | Up | Up | Up | yes |
| N315-SA2489 | | HP, similar to high-affinity | Up | Up | Up | yes^{d} |
| | | nickel-transport protein | | | | |
| N315-SA2491 | | Conserved HP | Up | Up | Up | yes |
| N315-SAS023 | | HP, similar to thioredoxin | 2.1 | 4.6 | 3.2 | |
| N315-SAS049 | | HP | Up | Up | Up | yes^{d} |
| N315-SAS053 | | HP | 4.0 | 12.8 | 2.1 | yes^{d} |
| N315-SAS056 | | HP | 2.0 | 5.7 | 1.9 | yes |
| N315-SAS068 | | HP | 5.2 | 5.7 | 3.3 | yes |
| N315-SAS082 | | HP | Up | Up | Up | |
| N315-SAS083 | | HP | Up | Up | Up | |
| N315-SAS089 | | HP | 2.6 | 5.7 | 2.3 | |
| COL-SA0866 | | HP | Up | Up | Up | |
| COL-SA1046 | | HP | 6.6 | 12.0 | 9.0 | yes |
| COL-SA2012 | | HP, acetyltransferase (GNAT) | 3.8 | 2.9 | 2.0 | |
| | | family | | | | |
| COL-SA2013 | | HP | Up | Up | Up | |
| COL-SA2379 | | Conserved HP | 2.2 | 17.1 | 3.0 | |
| COL-SA2433 | | HP | 2.6 | 3.6 | 2.1 | yes^{d} |
| COL-SA2481 | | HP | Up | Up | Up | yes^{d} |
| COL-SA2595 | | HP | 2.3 | 4.1 | 2.1 | |
| COL-SA2631 | | Conserved HP | Up | Up | 3.8 | yes |
| AAB05395 | | HP, ORF 3 of the sarA locus | 11.8 | 46.6 | 6.8 | yes |
| CAB60754 | | HP | 32.1 | Up | 13.9 | yes |

^{a}Based on the published sequence of strain N315 (accession no. NC_002745). For genes not present in N315, the gene name and description given are from the COL genome, available from The Institute for Genomic Research Comprehensive Microbial Resource website (www.tigr.org), or the respective accession number. ABC, ATP binding cassette; GNAT, GCN5-related N-acetyltransferases; HP, hypothetical protein; MHC, major histocompatibility complex; PTS, phosphotransferase system.
^{b}Normalized values in the rsbU⁺V⁺W⁺sigB⁺ strain over values in the ΔrsbUVWsigB mutant. “Up” denotes genes highly downregulated in the ΔrsbUVWsigB mutant, such that the transcripts were below detectable levels and the fold change could not be accurately calculated.
^{c}Open reading frames preceded by an consensus sequence that resembles the σ^{B} consensus sequence for B. subtilis as described by Petersohn et al. (62). Only sequences deviating not more than three nucleotides from the consensus GttTww₁₂₋₁₅ gGgwAw (w = a, t) and lying within 500 bp upstream of predicted open reading frames were considered as σ^{B}-dependent promoters.
^{d}Open reading frames likely to form an operon.
^{e}References reporting an influence of σ^{B} on the respective gene or its gene product.

TABLE 10
Genes Downregulated by σ^{B}

| N315 | N315 | | Fold change^{b} | | | Regulated |
|---|---|---|---|---|---|---|
| ORF No.^{a} | gene^{a} | N315 description^{a} | COL | Newman | GP268 | by SarA^{d} |
| N315-SA2430 | aur | Zinc metalloprotease aureolysin | 7.4 | 6.1 | 9.1 | Down |
| N315-SA2411 | citM | HP, similar to magnesium citrate | Down | Down | 4.3 | |
| | | secondary transporter | | | | |
| N315-SA0820 | glpQ | Glycerophosphoryl diester | 3.6 | 2.6 | 1.9 | Down |
| | | phosphodiesterase | | | | |
| N315-SA1007 | hla | α-hemolysin precurser | 2.1 | 2.8 | 14.1 | Up |
| N315-SA2207 | hlgA | γ-hemolysin component A | 1.7 | 2.0 | 2.1 | |
| N315-SA2209 | hlgB | γ-hemolysin component B | 2.2 | 4.2 | Down | Up |
| N315-SA2208 | hlgC | γ-hemolysin component C | 2.0 | 4.7 | 4.1 | Up |
| N315-SA2463 | lip | Triacylglycerol lipase precursor | 2.0 | 6.2 | 2.0 | Up/Down |
| N315-SA0252 | lrgA | Holin-like protein LrgA | — | 5.8 | 9.4 | Up |
| N315-SA0253 | lrgB | Holin-like protein LrgB | — | 6.2 | 6.5 | Up/Down |
| N315-SA1812 | lukF | HP, similar to | 2.7 | 3.9 | Down | |
| | | synergohymenotropic toxin | | | | |
| | | precursor | | | | |
| N315-SA1813 | lukM | HP, similar to leukocidin chain | 3.8 | 4.8 | Down | |
| | | lukM precursor | | | | |
| N315-SA0746 | nuc | Staphylococcal nuclease | 29.7 | 5.1 | Down | Down |
| N315-SA0091 | plc | 1-phosphatidylinositol | Down | 3.9 | Down | Down |
| | | phosphodiesterase precurosr | | | | |
| N315-SA0963 | pycA | Pyruvate carboxylase | 2.3 | 1.9 | 2.3 | |
| N315-SA0259 | rbsD | Ribose permease | 2.9 | 2.8 | 1.5 | |
| N315-SA0258 | rbsK | Probable ribokinase | 2.8 | 2.3 | 1.3 | |
| N315-SA1758 | sak | Staphylokinase precursor | — | 2.7 | 7.0 | |
| | | (protease III) | | | | |
| N315-SA0128 | sodM | Superoxide dismutase | 4.6 | 2.0 | 2.8 | |
| N315-SA1631 | splA | Serine protease SplA | Down | 9.9 | Down | Up |
| N315-SA1630 | splB | Serine protease SplB | Down | 7.9 | Down | Up |
| N315-SA1629 | splC | Serine protease SplC | Down | Down | Down | |
| N315-SA1628 | splD | Serine protease SplD | Down | Down | Down | Up |
| COL-SA1865 | splE | Serine protease SplE | Down | Down | Down | |
| BAB95617_1 | splF | Serine protease SplF | — | Down | Down | |
| N315-SA0901 | sspA | Staphylococcal serine protease | 3.8 | 2.1 | 3.3 | Down |
| | | (V8 protease) | | | | |
| N315-SA0900 | sspB | Cysteine protease | 3.2 | 2.2 | 4.3 | Down |
| N315-SA0899 | sspC | Cysteine protease | 3.0 | 1.9 | 3.0 | Down |
| N315-SA2302 | stpC | HP, similar to ABC transporter | 6.3 | 2.3 | 4.0 | |
| N315-SA0022 | | HP, similar to 5′-nucleotidase | 2.6 | 1.8 | 3.3 | |
| N315-SA0089 | | HP, similar to DNA helicase | 2.4 | Down | 2.1 | |
| N315-SA0260 | | HP, similar to ribose transporter | 3.0 | 2.6 | 2.3 | |
| | | RbsU | | | | |
| N315-SA0270 | | HP, similar to secretory antigen | 4.6 | Down | Down | |
| | | precursor SsaA | | | | |
| N315-SA0272 | | HP, similar to transmembrane | 4.4 | Down | Down | |
| | | protein Tmp7 | | | | |
| N315-SA0276 | | Conserved HP, similar to | 3.7 | Up | — | |
| | | diarrhoeal toxin-like protein | | | | |
| N315-SA0285 | | HP | 2.6 | Down | 3.4 | |
| N315-SA0291 | | HP | 3.1 | — | 3.3 | |
| N315-SA0295 | | HP, similar to outer membrane | 4.9 | 3.6 | 10.4 | |
| | | protein precursor | | | | |
| N315-SA0368 | | HP, similar to proton/sodium- | 2.7 | 3.1 | 1.4 | |
| | | glutamate symport protein | | | | |
| N315-SA0841 | | HP, similar to cell surface protein | 5.7 | 3.4 | 2.2 | |
| | | Map-w | | | | |
| N315-SA0977 | | 29-kDa cell surface protein | 2.5 | 2.1 | 1.8 | |
| N315-SA1725 | | Staphopain, cysteine protease | 5.9 | 4.2 | 10.6 | Down |
| N315-SA1726 | | HP | 3.8 | 3.4 | 6.5 | |
| N315-SA1815 | | HP, similar to Na+/−transporting | Down | Down | Down | |
| | | ATP synthase | | | | |
| N315-SA1853 | | HP, simialr to DNA mismatch | 2.1 | Down | 4.0 | |
| | | repair protein MutS | | | | |
| N315-SA2132 | | HP, simialr to ABC transporter | 2.7 | Down | 2.3 | |
| | | (ATP-binding protein) | | | | |
| N315-SA2133 | | Conserved HP | 3.1 | Down | 3.2 | |
| N315-SA2303 | | HP, similar to membrane | Down | 1.8 | Down | |
| | | spanning protein | | | | |
| N315-SAS020 | | HP, similar to phosphoglycerate | 2.1 | 2.4 | 2.2 | |
| | | mutase | | | | |
| COL-SA0450 | | HP | 2.2 | 2.2 | 3.1 | |
| COL-SA1884 | | HP | 3.3 | Down | Down | |
| COL-SA2693 | | HP | 2.2 | 7.1 | 2.2 | |

^{a}Based on the published sequence of strain N315 (accession no. NC_002745). For genes not present in N315, the gene name and description given are from the COL genome, available from The Institut for Genomic Research Comprehensive Microbial Resource website (www.tigr.org), or the respective accession number. HP, hypothetical protein.
^{b}Normalized values in the ΔrsbUVWsigB mutant over values in the rsbU⁺V⁺W⁺sigB⁺ strain. “Down” denotes genes highly downregulated in the rsbU⁺V⁺W⁺sigB⁺ strain, such that the transcripts were below detectable levels and the fold change could not be accurately calculated.
^{c}References reporting an influence of σ^{B} on the respective gene or its gene product.
^{d}References reporting an influence of SarA on the respective gene or its gene product.

Transcriptional start point (tsp) determinations of the σ^{B}-driven sarC and clfA transcripts, coupled with σ^{B}-dependent in vitro transcription analyses of the asp23 P1 and the coa promoters, suggest that the promoter region of S. aureus σ^{B} regulated genes contains a consensus sequence that is highly similar to that of B. subtilis σ^{B} regulated genes. See Petersohn et al., supra. Similarity of the σ^{B} promoter consensus sequences of both species is further corroborated by the findings that the S. aureus asp23 P1 promoter is recognized by E-σ^{B} in B. subtilis, and that all proteins that were identified to be influenced by σ^{B} in S. aureus by a proteomic approach are encoded by genes harboring a nucleotide sequence resembling the B. subtilis σ^{B} promoter consensus. Most of the genes, identified as upregulated by σ^{B} in this study, were also preceded by nucleotide sequences resembling the σ^{B} promoter consensus of B. subtilis, either directly or as part of a putative operon. None of the genes identified to be down-regulated in a σ^{B} specific manner contained this sequence within their promoter region. Tsp determinations of several of these genes, including asp23 P1, csbD, and csb9, further validate the similarity of the σ^{B} promoter consensus.

Genes influenced by σ^{B} during early growth stages: The approach used in experiment one proved to be useful for the identification of a large number of σ^{B}-regulated genes (Tables 9 and 10). However, this strategy might miss σ^{B}-dependent genes that were expressed only during early growth stages. In a second approach, the transcriptional profiles of strain Newman and its ΔrsbUVWsigB mutant, IK184, were analyzed during several growth stages, e.g. 1, 3, 5, and 8 h after inoculation. Monitoring the transcriptional profiles during different growth stages confirmed almost all genes identified by experiment one to be σ^{B}-dependent. The experiment also enabled the identification of 23 additional ORFs to be positively regulated by σ^{B} (Table 11). The majority of these ORFs, represented by transcriptional profile type 1, were expressed primarily during early growth stages (1 and 3 h after inoculation), while no transcripts were detectable during later growth (5 and 8 h after inoculation). Members of this group include several putative virulence factors such as coa, encoding for staphylococcal coagulase, and fnb, encoding fibronectin binding protein A, which have been demonstrated to be influenced by σ^{B} and confirmed in this study. In addition, ORFs N315-SA0620, N315-SA2093, and N315-SA2332, which all are homologues of ssaA of Stapyhlococcus epidermidis, encoding the highly antigenic staphylococcal secretory antigen A were found to be influenced by σ^{B}. Most of the ORFs listed in Table 11 lacked a significant σ^{B} consensus promoter in their upstream regions, suggesting that σ^{B} indirectly regulates their transcript titers.

TABLE 11
Genes Upregulated by σ^{B} in Strain Newman During Early Growth Phase

| N315 | N315 | | Fold | σ^{B} | Expression |
|---|---|---|---|---|---|
| ORF No.^{a} | gene^{a} | N315 description^{a} | change^{b} | consensus^{c,d} | profile^{e} |
| N315-SA0222 | coa | Staphylocoagulase precursor | 2.4 | yes | 1 |
| N315-SA2291 | fnb | Fibronectin binding protein A | 2.5 | | 1 |
| N315-SA2356 | isaA | Immunodominant antigen A | 2.4 | | 1 |
| N315-SA0265 | lytM | Peptidoglycan hydrolase | 3.4 | yes | 1 |
| N315-SA2093 | ssaA | Secretory antigen precursor SsaA | 2.4 | | 1 |
| | | homolog | | | |
| COL-SA0857 | vwb | Secreted von Willebrand factor- | 2.6 | | 1 |
| | | binding protein | | | |
| N315-SA0336 | | HP | 2.1 | | 1 |
| N315-SA0612 | | Conserved HP | 3.1 | | 2 |
| N315-SA0620 | | Secretory antigen SsaA | 2.7 | | 1 |
| | | homologue | | | |
| N315-SA0903 | | Conserved HP | 2.5 | | 1 |
| N315-SA0937 | | Cytochrome D ubiquinol oxidase | 2.2 | | 1 |
| | | subunit 1 homolog | | | |
| N315-SA0938 | | Cytochrome D ubiquinol oxidase | 2.0 | | 1 |
| | | subunit II homolog | | | |
| N315-SA1275 | | Conserved HP | 2.6 | | 1 |
| N315-SA1898 | | HP, simialr to SceD precursor | Up | Yes | 1 |
| N315-SA2301 | | HP, similar to alkaline | 2.2 | | 2 |
| | | phosphatase | | | |
| N315-SA2310 | | Conserved HP | 2.0 | | 2 |
| N315-SA2321 | | HP | 2.3 | yes | 2 |
| N315-SA2332 | | HP, similar to secretory antigen | 2.8 | | 1 |
| | | precursor SsaA | | | |
| N315-SA2355 | | Conserved HP | 2.3 | Yes | 1 |
| N315-SA2378 | | Conserved HP | 2.5 | | 1 |
| N315-SA2447 | | HP, similar to streptococcal | Up | yes | 2 |
| | | hemagglutinin protein | | | |
| N315-SAS051 | | HP | 2.1 | | 2 |
| COL-SA0210 | | HP | Up | | 1 |

^{a}Based on the published sequence of strain N315 (accession no. NC_002745). For genes not present in N315, the gene name and description given are from the COL genome, available from The Institut for Genomic Research Comprehensive Microbial Resource website (www.tigr.org), or the respective accession number. ABC, ATP binding cassette; GNAT, GCN5-related N-acetyltransferases; HP, hypothetical protein; MHC, major histocompatibility complex; PTS, phosphotransferase system.
^{b}Normalized values in the Newman strain over values in the ΔrsbUVWsigB mutant IK184. “Up” denotes genes highly downregulated in IK184, such that the transcripts were below detectable levels and the fold change could not be accurately calculated.
^{c}Open reading frames preceded by an consensus sequence that resembles the σ^{B} consensus sequence for B. subtilis as described by Petersohn et at. (62). Only sequences deviating not more than three nucleotides from the consensus GttTww₁₂₋₁₅ gGgwAw (w = a, t) and lying within 400 bp upstream of predicted open reading frames were considered as σ^{B}-dependent promoters.
^{d}Open reading frames likely to form an operon.
^{e}References reporting an influence of σ^{B} on the respective gene or its gene product.

Transcript titers of a number of ORFs was not only increased in the wild-type strain during early growth (1 h after inoculation), but was found to be further enhanced during late growth (8 h after inoculation) as represented by transcription profile type 2. It is conceivable that the expression of these ORFs is again influenced indirectly by σ^{B}, for example, via regulator(s), which are mainly active during the late growth phase. The increase in expression observed for these ORFs during the early growth phase may be due to a carry-over of the regulators that were produced during late growth in the pre-culture and may be still active even one hour after inoculation.

Functional classification of ORFs influenced by σ^{B}: The ORFs influenced by σ^{B} represent all functional categories, e.g. (i) cell envelope and cellular processes, including cell wall production, transport, signal transduction, membrane bioenergetics, and protein secretion; (ii) intermediary metabolism, including carbohydrate metabolism, glycolytic pathways, TCA cycle, amino acid and lipid metabolism; (iii) information pathways, including DNA modification and repair, RNA synthesis, and regulation; (iv) other functions, such as adaptation to atypical conditions or detoxification; and (v) ORFs similar to proteins with unknown function. The latter group alone comprises 100 out of the 251 ORFs regulated by σ^{B}, representing a large reservoir of potential factors that might be responsible for phenotypic properties of S. aureus associated with σ^{B} activity, such as the development of resistance to methicillin, glycopeptides and hydrogen peroxide that have not been associated with specific genes.

Chromosomal distribution of σ^{B}-regulated genes: The ORFs that are positively controlled by σ^{B} are not evenly distributed over the S. aureus chromosome but rather are overabundant in the genomic regions that are close to the origin of replication (oriC). While 77 out of 828 ORFs (9.3%) or 69 out of 861 ORFs (8%) encoded by the genome fragments 1 and 3, corresponding to position 1 to 937,880 and 1,875,761 to 2,813,641, respectively, are influenced by σ^{B}, only 12 out of 816 (1.5%) of the ORFs encoded by genomic region 2 (position 937,880 to 1,875,760) that is most distal to oriC, are controlled by σ^{B}. The majority of genes/operons in these segments are oriented with respect to oriC in a manner that minimizes collisions between the transcribing RNA polymerase and the replication apparatus. Thus, 71.5% of all genes, and 77% of the σ^{B}-regulated ORFs, located on genome fragment 1 are encoded by the clockwise replicating strand, and 72.8% of all genes and 72.5% of the σ^{B}-regulated ORFs located on genome fragment 3 are encoded by the counterclockwise strand. It has been suggested that the location of a gene relative to oriC can affect its level of expression. Genes located near the point of origin of replication are present in higher numbers in a rapidly growing cell than those near the terminus, which may be of importance for those genes that are controlled by promoters operating near the maximum possible frequency.

Putative regulators acting downstream of σ^{B}: A significant number of ORFs (50 out of 176 of experiment one, and 17 out of 23 of experiment two) found to be upregulated by σ^{B}, were not preceded by nucleotide sequences resembling the σ^{B} promoter consensus. Some of these genes were expressed only in sigB⁺ strains. It is possible that these ORFs were transcribed by the direct action of E-σ^{B}, despite of the lack of an obvious σ^{B} promoter consensus. Alternatively, it is possible that σ^{B} controls the expression of a regulator(s), which would subsequently promote the expression of these genes. Promising candidates for such a scenario are the putative regulator homologues YabJ and SpoVG (N315-SA0455/6), which may be co-transcribed, and were found to be controlled by σ^{B} Tsp determination of the yabJ transcript by S1 mapping confirmed that yabJ-spoVG expression is driven by σ^{B}, YabJ belongs to the highly conserved family of YigF proteins, which have been suggested to influence a variety of biological processes. YabJ of B. subtilis was found to have a role in the repression of purA by adenine. spoVG, encoding the stage V sporulation protein G, was the first developmentally regulated gene that was cloned from B. subtilis, and its regulation has been investigated intensively. However, little is known about the function of this protein. A mutation in spoVG was shown to impair sporulation of B. subtilis, apparently as a result of disintegration of an immature spore cortex. More recent results suggest that SpoVG interferes with or is a negative regulator of the pathway leading to asymmetric septation. In addition to S. aureus, spoVG homologues have been found in the genomes of several bacteria, such as Archeoglobus fulgidus, Borrelia burgdorferi, Listeria monocytogenes, and S. epidermidis, none of which produce spores. Thus, the SpoVG homologues of these organisms may mediate functions other than sporulation. Inactivation of spoVG in a methicillin-resistant S. epidermidis (MRSE) drastically decreased methicillin resistance and the formation of a biofilm. Interestingly, both attributes have also been linked positively to σ^{B} activity in S. aureus (65, 80). Attempts to inactivate the S. aureus yabJ and spoVG homologues are currently ongoing in order to elucidate their roles in this organism.

Another potential regulator, acting downstream of σ^{B}, is the gene product of ORF N315-SA1961, a homologue of the BglG/SacY family of transcriptional anti-terminators (ATs). ATs are regulatory protein factors that bind to specific sites in the nascent mRNA in order to prevent premature termination of gene transcription and to stimulate elongation by RNA polymerase. Expression of N315-SA1961 was found to be highly upregulated in strains harboring an intact sigB operon (Table 9), and the ORF is preceded by a nucleotide sequence that matches the proposed σ^{B} promoter consensus, indicating that the BglG/SacY homologue is controlled directly by σ^{B}.

Influence of σ^{B} on known regulatory elements: S. aureus possesses an array of virulence factor regulatory elements, such as two-component signal transduction systems and winged-helix transcription-regulatory proteins. Presumably these elements interact to influence different networks of virulence factors on an as-needed basis, thereby providing cells with the necessary arsenal of virulence determinates to respond to environmental changes or stimuli. The data presented here indicate that three of these virulence regulators, sarA, sarS and arlRS are upregulated by σ^{B}. Transcription of other well-studied virulence regulators, such as Sae and Rot, were not significantly influenced by σ^{B} in this study.

The staphylococcal accessory regulator A, SarA, a member of the winged-helix transcription proteins is encoded by the sar locus. Although the expression of the sar locus is in-part controlled by the action of σ^{B}, it is still a matter of debate whether σ^{B} has a positive or negative effect on the overall level of SarA production. Much of what is published regarding the influence of σ^{B} on SarA expression is difficult to interpret because most of these studies were done in strains, such as RN6390 and 8325-4, that harbor mutations in rsbU, the positive activator of σ^{B}, rendering them sigB deficient. The discrepancies between the positive influence of σ^{B} on SarA production observed by Gertz, et al., J. BACTERIOL., 182: 6983-6991 (2000), in a proteomic approach and by Bischoff, et al, J. BACTERIOL. 183: 5171-5179 (2001), via reporter gene fusion experiments, versus the observed down-regulatory effect of σ^{B} on SarA production reported by Manna, et al., J. BACTERIOL., 180: 3828-3836 (1998) and Cheung, et al., INFECT. IMMUN., 67: 1331-1337 (1999) might be explained by the fact that, in the latter studies, an rsbU mutant was used as parental strain to compare it with its respective sigB mutant. However, this explanation seems not to be able to account for the findings of Horsburgh, et al., J. BACTERIOL., 184: 5457-5467 (2002), who did not observe any influence of σ^{B} on SarA production either at the transcriptional or protein level. The transcriptional profiling data presented here suggests that σ^{B} increases the expression of the sar locus (Table 9), for instance, during later growth stages (5 and 8 h after inoculation). Moreover, a direct correlation between the increase in SarA transcript levels and an increase in SarA protein is indirectly suggested by the findings that expression of four major extracellular proteases of S. aureus (staphylococcal serine protease V8 [SspA], cysteine protease [SspB], metalloprotease aureolysin [Aur], and staphopain [Scp]) is significantly decreased in sigB⁺ strains (Table 10). It was recently demonstrated that transcription of these protease genes was suppressed due to increased σ^{B}-dependent expression of SarA. This is further supported by the findings that several of the ORFs found to be downregulated by σ^{B}, such as glpQ, encoding glycerophosphoryl diester phosphodiesterase, nuc, encoding staphylococcal thermonuclease, and plc, encoding a 1-phosphatidylinositol phospodiesterase precursor, have been demonstrated to be downregulated by SarA. It is possible that the increase in expression of these genes found in the ΔrsbUVWsigB mutants is due to a decreased production of SarA. Although appealing, this assumption remains speculative, as previous studies used the rsbU defective RN6390 lineage as genetic background for their analyses, leaving it open to question what might happen with respect to the sarA regulon in strains carrying an intact sigB operon. The genetic background chosen may also explain the observed discrepancy that several of the genes listed in Table 10 were found to be downregulated by σ^{B}, but upregulated by SarA. Support for such a process is conferred by the observations that RNAIII expression of the agr locus is promoted by SarA, but decreased by σ^{B} in an unidentified way that is, however, supposed to be independent from SarA

Expression of a second winged-helix transcription protein, SarS (syn. SarH1), belonging to the family of SarA homologues, was shown to be influenced by σ^{B}. This was confirmed in two of the three backgrounds analyzed in this study (Table 9). Interestingly, no difference in sarS expression was observed when comparing strain Newman and its ΔrsbUVWsigB mutant either in the microarray experiments (Table 9) or by Northern blot analysis (data not shown), further demonstrating that strain to strain differences influence regulon constituents. Sequencing of the σ^{B} promoter regions of sarS of strains Newman and GP268 did not reveal any difference between the respective regions (which were identical with the N315 region corresponding to nucleotides 125,868 to 126,073 of GenBank accession AP003129), leaving the question open as to why expression of sarS in Newman is not affected by σ^{B}.

The third known virulence regulatory element observed to be influenced by σ^{B} was arlRS, encoding a two-component signal transduction system that influences adhesion, autolysis, and extracellular proteolytic activity of S. aureus. More recently, it was also demonstrated to decrease expression of the agr locus, while increasing the expression of SarA. The data obtained from experiment two suggest that arlRS of strain Newman is upregulated by σ^{B}. However, arlRS did not show up in experiment one as influenced by σ^{B} either in strain COL or strain GP268, and is not preceded by a σ^{B} consensus promoter.

Recent results suggest that expression of RNAIII, the effector molecule of the agr locus, is negatively influenced by σ^{B}. However, results of the two experiments presented here did not effectively corroborate these observations, as although slight differences in RNAIII transcription were detectable between wild-type strains and their respective ΔrsbUVWsigB mutants, changes in expression were not determined to be significant. RNAIII is by far the most prominent RNA molecule produced by S. aureus during later growth stages. As a result, the RNAIII transcript levels of the wild-type strains already reached amounts that saturated the RNAIII specific target oligonucleotides represented on the microarray, thus impeding the detection of differences in RNAIII transcript levels that might be present between the strain pairs analyzed.

Influence of σ^{B} on the expression of virulence determinants: Previous studies demonstrated that σ^{B} influences the expression of various factors associated with virulence and pathogenicity of S. aureus. However, in vivo studies have failed to demonstrate an effect of σ^{B} on virulence of S. aureus. Alternatively, σ^{B} may play a role in pathogenesis, however, the effects of σ^{B} mediated virulence mechanisms do not play a role in the models chosen in those experiments.

Analysis of the microarray data suggests that σ^{B} influences the expression of a large number of virulence genes in S. aureus. Many of these are reported here as genes that are altered transcriptionally by a σ^{B}. By comparing the expression profiles of these virulence genes a pattern has emerged; most of the exoenzymes and toxins produced by S. aureus were negatively influenced by σ^{B}, while expression of several adhesins were found to be increased by σ^{B}. The function of σ^{B} in virulence factor production therefore seems to be opposite to that of RNAIII, which is known to act as a negative regulator of cell wall proteins and a positive regulator of exoenzymes and toxins in a growth phase-dependent manner (Table 12). The decreased amounts of exoprotein and toxin transcripts observed in wild type strains compared to their respective mutants may in part be a consequence of lower RNAIII transcript levels that are present in strains harboring an intact sigB operon.

TABLE 12
Influence of σ^{B} on Virulence Determinants Regulated by the agr Locus

| | gene name | agr | σ^{B} |
|---|---|---|---|
| Aureolysin | aur | + | − |
| Capsular polysaccharide synthesis | cap5J | + | + |
| enzyme 5J | | | |
| Clumping factor B | clfB | + | Ø |
| Coagulase | coa | − | + |
| Cystein protease | sspC | + | − |
| Enterotoxin B | sea | + | Unknown |
| Enterotoxin C | seb | + | Unknown |
| Exotoxin 2 | set8 | + | Unknown |
| Factor effecting methicillin | femB | + | Ø |
| resistance B | | | |
| Fibronectin-binding protein A | fnbA | − | + |
| Fibronectin-binding protein B | fnbB | − | Ø |
| Glycerol ester hydrolyase | geh | + | − |
| α-hemolysin | hla | + | − |
| β-hemolysin | hlb | + | −¹ |
| γ-hemolysin | hlgBC | + | − |
| δ-hemolysin | hld | + | Ø |
| Hyaluronate lyase | hysA | + | Ø |
| Lipase | lip | + | − |
| LrgAB (holin-like proteins) | lrgAB | + | − |
| Myosin-crossreactive antigen | (N315-SA0102) | − | + |
| Phosphatidylinositol-specific | plc | + | − |
| phospolipase C | | | |
| Protein A | spa | − | Ø |
| Secretory antigen A | ssaA | − | + |
| Serine protease A, B, D, and F | splA, B, D, F | + | − |
| Staphylokinase | spc | + | − |
| TSST-1 | tst | + | Unknown |
| V8 protease | sspA | + | − |

Genes that are regulated converse by agr and σ^{B} are highlighted.
¹based on the hlb transcript levels detected in strains COL and IK183.

The finding that expression of so many virulence genes are significantly altered by σ^{B}, warrants further investigation to elucidate its role in infectivity of S. aureus in additional models of infection. To date, little is known about the expression or activity of σ^{B} during the course of infection. S. aureus is known for its ability to cause a variety of unrelated infections. It is feasible that the σ^{B}-dependent downregulation of toxins and exoenzymes, combined with the simultaneous upregulation of adhesins, may enable S. aureus to cause very specific host-pathogen interactions that have not been investigated to date. Recent results indicate that σ^{B} involved in processes that are important for biofilm formation. Therefore a comparison of the transcription profile of biofilm cells to the results obtained herein may identify genes that are essential for biofilm formation. Additionally, based on the virulence factor pattern caused by σ^{B}, it is tempting to speculate that this alternative transcription factor may also be an important player during nasal colonization, thereby promoting adherence to the host cell matrix without evoking an inflammatory response. Investigations are ongoing to address these questions. It is also quite possible that in vivo conditions leading to S. aureus stress, including those of high temperature at the site of infection, may induce the stress responsive σ^{B} factor. Under such conditions, when the host is trying to mount an immune response at the site of infection it could be more beneficial for the bacterium to produce cell surface components that are involved in camouflaging the organism from the host's defense than exoproteins.

The Example was designed to extensively characterize the genes that are regulated by the alternative sigma factor σ^{B} during standard laboratory growth conditions. Under these conditions, an X fold increase in sigB expression and >100-fold increase in the sigB regulated gene asp23 was observed. In addition, very stringent criteria were used for the identification of σ^{B} regulated genes: (1) transcripts demonstrated the same σ^{B} dependent phenotype in at least two out of the three genetic backgrounds tested, and (2) transcripts passed strict statistical cut-off values. Based on these criteria there was a high correlation between the genes identified in this Example and other recorded results. As a consequence, it is likely that the microarray methodology used accurately identified the genes belonging to the σ^{B} regulon of the strains analyzed. While defining the sigB regulon, a distinguishable pattern among virulence factors were observed. Subsequent studies that have focused on two S. aureus adhesions (clfA and fnbA) have confirmed that each gene is indeed regulated in a σ^{B} dependent manner and further validated the methodology used.

The finding that σ^{B} downregulates the transcription of secreted—but upregulates cell surface-virulence factors is in direct contrast to the observations of Kupferwasser, et al., J. CLIN. INVEST, 112: 222-233 (2003). In that study it was found that salicyclic acid mildly induces asp23 (1.9-fold) and corresponds to both the down regulation of certain cell surface adhesions and upregulation of secreted proteases. Based on the low induction rate of asp23 it is difficult to reconcile whether the virulence factor effects seen in that study are directly mediated by σ^{B} verses another salicyclic acid responsive process or a combination of the two. It also raises the question whether low to moderate levels of sigB produce a much different physiological phenotype than the levels tested here. It is also possible that salicyclic acid and other stresses that have been shown to modulate sigB activity direct the expression of portions of the sigB regulon. Having more completely characterized the σ^{B} regulon will allow subsequent experiments to fully address these questions and further understand the effects, if any, the σ^{B} regulon plays in pathogenesis.

### Example 10

### Staphylococcus aureus Nucleic Acid Arrays in Genotyping and Genetic Composition Analysis

Understanding the relatedness of strains within a bacterial species is important for monitoring reservoirs of antimicrobial resistance and for epidemiological studies. Pulsed-field gel electrophoresis (PFGE), ribotyping and multilocus sequence typing (MLST) are commonly used for this purpose. However, these techniques are either non-quantitative or provide only a limited estimation of strain relatedness. Moreover, they cannot extensively define the genes that constitute an organism. In this example, 21 oxacillin resistant Staphylococcus aureus (ORSA) isolates, representing eight major ORSA lineages, and each of the 7 strains for which complete genomic sequence is publicly available were genotyped using the nucleic acid array of Example 1. Strains were also subjected to PFGE and ribotyping analysis. The nucleic acid array results provided a higher level of discrimination among isolates than either ribotyping or PFGE, although strain clustering was similar among the three techniques. In addition, nucleic acid array signal intensity cut-off values were empirically determined to provide extensive data on the genetic composition of each isolate analyzed. Using this technology it was shown that strains could be examined for each element represented on the nucleic acid array including: virulence factors, antimicrobial resistance determinants, and agr-type. These results were validated by PCR, growth on selective media and detailed in silico analysis of each of the sequenced genomes. Therefore, nucleic acid arrays can provide extensive genotyping information for S. aureus strains and may play a major role in epidemiological studies in the future where correlating genes with particular disease phenotypes is critical.

Materials and Methods

DNA isolation and labeling. S. aureus strains were grown overnight in Brain Heart Infusion (BHI) medium in ambient air at 370C with vigorous aeration. For chromosomal isolation 1.5 ml of an overnight culture in BHI was placed in a 1.5 ml Eppendorf tube and was centrifuged for 5 min at 4° C. at high-speed in a table-top centrifuge. Supernatants were discarded and cell pellets were resuspended in an equal volume of ice-cold TE buffer (10 mM Tris, 1 mM EDTA; pH 8.0). Suspensions were then placed in 2-ml Lysing Matrix tubes (Bio 101; Vista, Calif.). Cells were lysed by shaking in a FP120 reciprocating shaker (Bio 101) two times at 6000 rpm for 20 s and cell debris was pelleted by centrifugation at high speed in a table top centrifuge for 10 min. Chromosomal and plasmid DNA was then purified from the supernatant on a Qiagen DNA tissue easy column (Valencia, Calif.), following the manufacturer recommendations for bacterial DNA purification. 2 μg of purified DNA was subjected to electrophoresis on a 0.8% native agarose gel to assess DNA integrity. For DNA labeling 5 μg of purified DNA was incubated at 90° C. for 3 min then plunged into an ice-bath followed by standard DNA fragmentation and labeling procedures according to the manufacturer's (Affymetrix Inc.,) instructions for labeling mRNA for antisense prokaryotic arrays. 1.5 μg of labeled DNA was hybridized to a nucleic acid array and was processed as per the manufacturer's protocol for GeneChip® hybridization and washing. Nucleic acid arrays were scanned, and signal intensities for elements tiled onto each nucleic acid array were normalized to account for loading errors and differences in labeling efficiencies by dividing each signal intensity by the mean signal intensity for an individual nucleic acid array. Results were analyzed using GeneSpring version 6.1 (Silicon Genetics, Calif.) and Spotfire version 7.0.

Ribotyping and PFGE: Strains were subjected to PFGE, as described in, McDougal, et al., J. CLIN. MICROBIOL., 41: 5113-5120 (2003). Ribotyping was performed using the RiboPrinter® system (Qualicon, Wilmington, Del.) according to the manufacturer's instructions. Each strain was analyzed using two restriction enzymes, EcoRI and PvuII. Computer-generated riboprints for each strain were assigned to an EcoRI or PvuII ribogroup by the software, and then visually inspected for correct assignment into ribogroups. Individual ribotypes were assigned to a strain based on identity of ribogroups for both restriction enzyme

Results

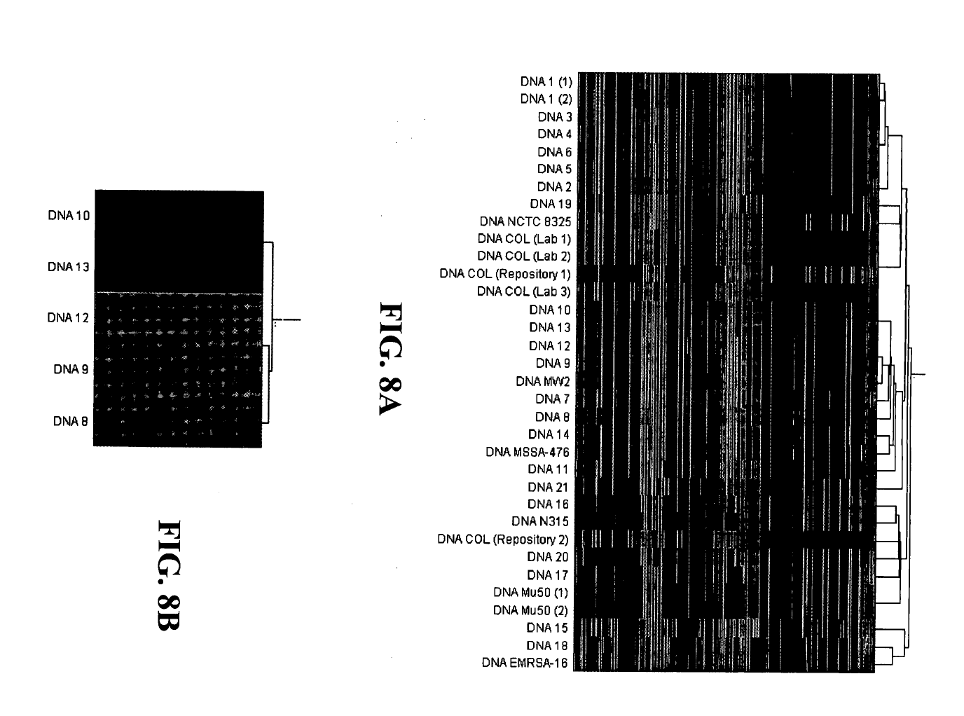

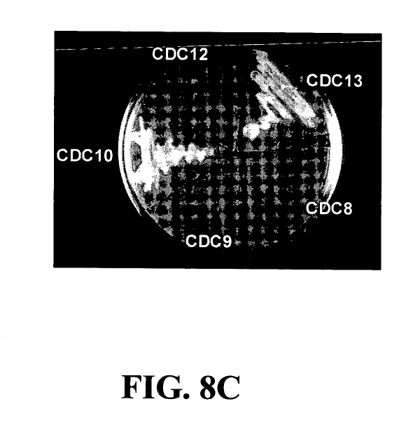

In addition to simultaneously providing an ability to obtain gene-by-gene information for a strain under investigation, the nucleic acid array of Example 1 was used to determine the relatedness of each strain that was being analyzed. This was accomplished by using hierarchical clustering to develop a dendogram that compared the normalized signal intensity of each qualifier for a given strain to the signal intensity of the same qualifier across all strains analyzed (FIG. 8A). Using this approach, strains that have similar signal intensities for all qualifiers are positioned closer together on the dendogram than strains with divergent genomic compositions (differing signal intensities for the same qualifiers).

The data were validated by several observations. First, as shown in FIG. 8A, strains 1, 10/13 (both are the same strain), COL and Mu50 were independently tested multiple times and replicates were considered more closely related than other strains analyzed. Isolates 10 and 13 are the same strain; they were included twice to serve as a control for this analysis. Second, in silico comparisons demonstrated that among sequenced strains: (1) MW2 is most closely related to MSSA-476, (2) Mu50 is closely related to N315 and moderately related to EMRSA-16, and (3) COL is closely related to NCTC 8325. Each of these relationships was detected in the dendrogram (FIG. 8A). Finally, both ribotyping and PFGE clustering agreed with the dendrogram derived from nucleic acid array data (Table 13).

TABLE 13
Ribotyping, Nucleic Acid Array and PFGE Genotyping Results

| | Nucleic | | |
|---|---|---|---|
| Strain | Acid Array | Ribotype | PFGE |
| CDC 1 | 1.1 | XII | USA300 (0.0114) |
| CDC 3 | 1.1 | XII | USA300 (0.0114) |
| CDC 4 | 1.1 | XII | USA300 (0.0114) |
| CDC 6 | 1.1 | XII | USA300 (0.0114) |
| CDC 5 | 1.1 | XII | USA300 (0.0114) |
| CDC 2 | 1.2 | XII | USA300 (0.0047) |
| CDC 19 | 1.3 | XII | USA500 TYPE (.0004) |
| NCTC 8325 | 1.4 | XIII | N.D. |
| COL (Lab 1) | 1.5 | IX | N.D. |
| COL (Lab 2) | 1.5 | N.D. | N.D. |
| COL (Repository-1) | 1.5 | N.D. | N.D. |
| COL (Lab 3) | 1.5 | N.D. | N.D. |
| CDC 10 | 2.1 | XI | USA400 (0.0051) |
| CDC 13 | 2.1 | XI | USA400 (0.0051) |
| CDC 12 | 2.2 | XI | USA400 (0.0051) |
| CDC 9 | 2.2 | XI | USA400 (0.0051) |
| MW2 | 2.3 | XI | N.D. |
| CDC 7 | 2.4 | IV | USA400 (0.0199) |
| CDC 8 | 2.5 | XI | USA400 (0.0051) |
| CDC 14 | 2.6 | X | USA400 (0.0172) |
| MSSA-476 | 2.7 | XI | N.D. |
| CDC 11 | 2.8 | XI | USA400 (0.0080) |
| CDC 21 | 2.9 | VI | USA700 TYPE (0.0097) |
| CDC 16 | 3.1 | V | USA100/800 |
| N315 | 3.2 | N.D. | N.D. |
| COL (Repository-2) | 3.3 | N.D. | N.D. |
| CDC 20 | 3.4 | II | USA600 TYPE |
| CDC 17 | 3.5 | VII | USA100-B (0.0022) |
| Mu50 (1) | 3.6 | N.D. | N.D. |
| Mu50 (2) | 3.6 | N.D. | N.D. |
| CDC 15 | 4.1 | III | USA600 (0.0121) |
| CDC 18 | 4.2 | VIII | USA200 TYPE |
| EMRSA-16 | 4.3 | I | N.D. |
| Ribotyping, GeneChip and PFGE results are shown for each strain. Strains were observed to fit into 4 major clusters by nucleic acid array analysis (FIG. 8A.). Individual strains within each of these clusters are further distinguished. For example, nucleic acid array profiles 2.2 and 2.3 are different strains within cluster number two. Strains with the same profile numbers are identical. Ribotyping results distinguished strains as belonging to one of 12 different ribogroups (I-XII). | | | |
| # PFGE results demonstrated that strains belonged to 8 different groups (USA100-USA800; 80% identity cut-off). Number in parenthesis represents the strain's identification number. Strains with same identification number are considered identical. | | | |

Despite the similarity between the three-genotyping approaches, nucleic acid array results appeared to be the most discriminative. For instance, ribotyping data indicated that 7 strains fit into ribogroup XII and 8 strains belonged to ribogroup XI. As shown in Table 13, both PFGE and nucleic acid array-based typing further distinguished members of each ribogroup into subgroups. In the case of ribogroup XII, PFGE and nucleic acid array analysis further distinguished strains into identical subgroups. However, five strains from ribogroup XI were considered identical by PFGE (isolates 8, 9, 10, 12 and 13), but were further distinguished as 3 separate strains by nucleic acid array (Table 4; FIGS. 8A and 8B). To determine which typing method provided more accurate results, adjusted-call determinations were compared for all qualifiers across these 5 strains. As shown in FIG. 8B, 36 genes including the antimicrobial resistance determinants ermA, bleO and aadA were considered to be present in strains 10 and 13, but absent from strains 9, 12, and 8. To determine if these nucleic acid array predictions were correct, strains were tested for growth on antibiotic-containing agar plates. Strains 10 and 13 formed colonies on plates containing kanamycin, whereas isolates 8, 9 and 12 did not, confirming that the five strains are not identical in genetic composition (FIG. 8C). In addition, adjusted detection call predictions indicated that 31 genes were present in strains 9 and 12 but absent from strains 10 and 13. Collectively these results suggested that nucleic acid array-based genotyping was more discriminative than both ribotyping and PFGE.

The nucleic acid array technology is expected to provide novel information about S. aureus pathogenesis, antimicrobial resistance, and vaccine tolerance. For example, studies can now be carried out to identify whether the Panton-Valentine leukocidin virulence factor genes are also present in health care institution-associated strains. Such a study will be helpful in defining whether a subset of genes can distinguish community associated—from nosocomial—ORSA strains. Defining the entire repertoire of genes that are conserved across diverse CO-ORSA strains may also clarify how the proteins that they encode influence the prevalence of ORSA within the community.

Several genes have been linked to a particular type of S. aureus infection, such as tst with toxic shock syndrome and exofoliative toxins with scaled-skin syndrome (SSS). It is expected that the nucleic acid array technology will also provide the ability to associate subsets of S. aureus genes with particular types of infections. Moreover, because nucleic acid arrays can contain alleles of many genes, the potential exists to associate a particular phenotype with a gene allele. Studies evaluating agr-types have demonstrated that allelic types do influence pathogenesis and thus their identification is important for epidemiological studies. Many clinical isolates are agr group-1. agr group-3 has been associated with CA-MRSA, group-2 has been linked to intermediate glycopeptide resistance, and group-4 has been associated with exfoliative toxin producing strains. The nucleic acid array technology can be used to analyze the association of specific agr-type(s), and other genes/alleles, with disease causing strains.

Furthermore, the nucleic acid array approach can allow for one to determine whether a group of similar strains under investigation are clonal or slightly divergent in genetic composition. This distinction is an important aspect of monitoring strain outbreaks. The technology can also be used for analyzing the acquisition of antimicrobial resistance determinants and may provide a means to evaluate whether other genetic determinants confer a predisposition, or contribute to, the development of resistance.

In many cases, MLST, ribotyping, and PFGE provide the level of discrimination needed to monitor strains circulating throughout the community and healthcare environments. These techniques are rapid, do not require extensive analysis, and can be accomplished at a fraction of the cost associated with microarrays. However, none of these methods allows one to simultaneously define the genes that constitute the organism(s) under investigation on a genome scale. In addition to the uses described above, the present invention contemplates the approach described herein to be helpful in characterizing isolates within the same ribo-, MLST- or PFGE-group, or in studies where further characterization is needed.

The foregoing description of the present invention provides illustration and description, but is not intended to be exhaustive or to limit the invention to the precise one disclosed. Modifications and variations consistent with the above teachings may be acquired from practice of the invention. Thus, it is noted that the scope of the invention is defined by the claims and their equivalents.

What is claimed is:

1. A nucleic acid array comprising a plurality of polynucleotides and a plurality of discrete regions, wherein each of said plurality of polynucleotides is stably attached to a respective discrete region selected from said plurality of discrete regions, and wherein the plurality of polynucleotides includes two or more different polynucleotides, each of which is specific to a different respective strain selected from a plurality of strains of a non-viral species.

2. The nucleic acid array according to claim 1, wherein said plurality of polynucleotides includes at least one polynucleotide probe which is common to said plurality of strains.

3. The nucleic acid array according to claim 2, wherein the non-viral species is a bacterium.

4. The nucleic acid array according to claim 3, wherein the bacterium is Staphylococcus aureus.

5. The nucleic acid array according to claim 4, wherein said plurality of strains comprises two or more Staphylococcus aureus strains selected from the group consisting of COL, N315, Mu50, EMRSA-16, MSSA-476, MW2, and 8325.

6. The nucleic acid array according to claim 4, wherein said plurality of polynucleotides includes at least 100 polynucleotides, each of which is capable of hybridizing under stringent or nucleic acid array hybridization conditions to a different respective sequence selected from SEQ ID NOs: 1 to 7,852, or the complement thereof.

7. The nucleic acid array according to claim 4, wherein said plurality of polynucleotides includes at least 1,000 polynucleotides, each of which is capable of hybridizing under stringent or nucleic acid array hybridization conditions to a different respective sequence selected from SEQ ID NOs: 1 to 7,852, or the complement thereof.

8. The nucleic acid array according to claim 4, wherein said plurality of polynucleotides includes six polynucleotides, each of which is specific to a different respective Staphylococcus aureus strain selected from the group consisting of COL, N315, Mu50, EMRSA-16, MSSA-476, and 8325.

9. The nucleic acid array according to claim 8, wherein said plurality of polynucleotides includes a first set of polynucleotides, each of which is capable of hybridizing under stringent or nucleic acid array hybridization conditions to a different respective sequence selected from SEQ ID NOs: 3,817 to 7,852, or the complement thereof, and wherein said plurality of polynucleotides further includes a second set of polynucleotides, each of which is capable of hybridizing under stringent or nucleic acid array hybridization conditions to a different respective sequence selected from SEQ ID NOs: 1 to 3,816, or the complement thereof.

10. The nucleic acid array according to claim 9, wherein each of said first and second sets comprises at least 100 polynucleotides.

11. The nucleic acid array according to claim 1, wherein said non-viral species is Staphylococcus aureus, and said plurality of polynucleotides includes at least 100 polynucleotides, each of which is capable of hybridizing under stringent or nucleic acid array hybridization conditions to a different respective sequence selected from SEQ ID NOs: 7,704, or the complement thereof.

12. The nucleic acid array according to claim 11, wherein said non-viral species is Staphylococcus aureus, and said plurality of polynucleotides includes at least 1,000 polynucleotides, each of which is capable of hybridizing under stringent or nucleic acid array hybridization conditions to a different respective sequence selected from SEQ ID NOs: 7,704, or the complement thereof.

13. The nucleic acid array according to claim 11, wherein said plurality of polynucleotides comprises at least one oligonucleotide probe selected from SEQ ID NOs: 15,737.

14. The nucleic acid array according to claim 11, wherein said plurality of polynucleotides comprises at least probe for a Staphylococcus aureus gene selected from the group consisting of a virulence gene, an antimicrobial resistance gene, a multilocus sequence typing gene, a leukotoxin gene, an agrB gene, and a gene encoding a ribosomal protein.

15. A method comprising:
preparing a nucleic acid sample from a sample of interest; and
hybridizing the nucleic acid sample to the nucleic acid array of claim 1 to detect the presence or absence of a strain of said non-viral species.

16. A method comprising:
preparing a nucleic acid sample from a sample of interest; and
hybridizing the nucleic acid sample to the nucleic acid array of claim 4 to detect or monitor gene expression of a strain of said non-viral species.

17. A method comprising:
preparing a nucleic acid sample from a sample of interest; and
hybridizing the nucleic acid sample to the nucleic acid array of claim 1 to type a strain of said non-viral species.

18. A method of making a nucleic acid array, comprising the steps of:
selecting a plurality of polynucleotides, each of which is specific to a different respective strain selected from a plurality of strains of a non-viral species; and
attaching said plurality of polynucleotides to respective regions on one or more substrate supports.

19. A polynucleotide collection comprising at least one polynucleotide capable of hybridizing under stringent or nucleic acid array hybridization conditions to a respective sequence selected from SEQ ID NOs: 1 to 7,852, or the complement thereof.

20. A protein array comprising a plurality of probes, wherein each of said probes is specific to a different respective strain selected from a plurality of strains of a non-viral species, and each of said probes is capable of binding to a different respective protein of said non-viral species.
